(19) United States
(12) Patent Application Publication
(10) Pub. No.: US 2009/0048247 A1
(43) Pub. Date: Feb. 19, 2009

(54) CONDENSED ISOXALINE DERIVATIVES AS INHIBITORS OF PHOSPHODIESTERASE TYPE - IV

(76) Inventors: Venkata P. Palle, Haryana (IN); Sarala Balachandran, New Delhi (IN); Nidhi Gupta, New Delhi (IN); Nagarajan Muthukamal, Tanul Nadu (IN); Mandadapu Raghu Ramaiah, Andra Pradesh (IN); Manoj Kumar Khera, Haryana (IN); Lailt Kumar Baregama, Kapasan (IN); Vinayak Vasantrao Khairnar, Maharashtra (IN); Abhijit Ray, New Delhi (IN); Sunanda G. Dastidar, New Delhi (IN)

Correspondence Address: RANBAXY INC., 600 COLLEGE ROAD EAST, SUITE 2100, PRINCETON, NJ, 08540, US

(21) Appl. No.: 11/815,695

(22) PCT Filed: Feb. 13, 2006
(86) PCT No.: PCT/IB2006/000285

§ 371 Date: May 13, 2008

(30) Foreign Application Priority Data
Feb. 11, 2005 (IN) 303/DEL/2005

(51) Int. Cl. A61K 31/5377; C07D 498/10; A61K 31/424; A61P 31/18

(52) U.S. Cl. 514/233.5; 548/242; 514/379; 544/137

(57) ABSTRACT

The present invention relates to isoxazoline derivatives, which can be used as selective inhibitors of phosphodiesterase (PDE) type IV. In particular, compounds disclosed herein can be useful in the treatment of AIDS, asthma, arthritis, bronchitis, chronic obstructive pulmonary disease (COPD), psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis and other inflammatory diseases in a patient, particularly in humans. The present invention also relates to processes for the preparation of disclosed compounds, as well as pharmaceutical compositions thereof, and their use as phosphodiesterase (PDE) type IV inhibitors.

### FIELD OF THE INVENTION

The present invention relates to isoxazoline derivatives, which can be used as selective inhibitors of phosphodiesterase (PDE) type IV. In particular, compounds disclosed herein can be useful in the treatment of AIDS, asthma, arthritis, bronchitis, chronic obstructive pulmonary disease (COPD), psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis and other inflammatory diseases in a patient, particularly in humans. The present invention also relates to processes for the preparation of disclosed compounds, as well as pharmaceutical compositions thereof, and their use as phosphodiesterase (PDE) type IV inhibitors.

### BACKGROUND OF THE INVENTION

It is known that cyclic adenosine-3′,5′-monophosphate (cAMP) exhibits an important role of acting as an intracellular secondary messenger. The intracellular hydrolysis of cAMP to adenosine 5′-monophosphate (AMP) causes a number of inflammatory conditions, which include, but are not limited to, psoriasis, allergic rhinitis, shock, atopic dermatitis, Crohn's disease, adult respiratory distress syndrome (ARDS), eosinophilic granuloma, allergic conjunctivitis, osteoarthritis, and ulcerative colitis. Cyclic nucleotide phosphodiesterases (PDE), a biochemically and functionally, highly variable superfamily of the enzyme, is the most important factor in the control of cAMP (as well as of cGMP) levels. Eleven distinct families with more than 25 gene products are currently recognized. Although PDE I, PDE II, PDE III, PDE IV, and PDE VII all use cAMP as a substrate, only the PDE IV and PDE VII types are highly selective for hydrolysis of cAMP. Accordingly, inhibitors of PDE, particularly the PDE IV inhibitors, such as rolipram or Ro-1724, are known as cAMP-enhancers. Immune cells contain PDE IV and PDE III, of which PDE IV is prevalent in human mononuclear cells. Thus, the inhibition of phosphodiesterase type IV has been a target for modulation and, accordingly, for therapeutic intervention in a range of disease processes.

The initial observation that xanthine derivatives, theophylline and caffeine inhibit the hydrolysis of cAMP led to the discovery of the required hydrolytic activity in the cyclic nucleotide phosphodiesterase (PDE) enzymes. More recently, distinct classes of PDE have been recognized, and their selective inhibition has led to improved drug therapy. Thus, it was recognized that inhibition of PDE IV could lead to inhibition of inflammatory mediator release and airway smooth muscle relaxation.

3-Aryl-2-isoxazoline derivatives are known as anti-inflammatory agents and isoxazoline compounds are known as inhibitors of TNF release. However, there remains a need for new selective inhibitors of phosphodiesterase (PDE) type IV.

### SUMMARY OF THE INVENTION

The present invention provides isoxazoline derivatives, which can be used for the treatment of AIDS, asthma, arthritis, bronchitis, chronic obstructive pulmonary disease (COPD), psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis and other inflammatory diseases, and the processes for the synthesis of these compounds.

Pharmaceutically acceptable salts, pharmaceutically acceptable solvates, enantiomers, diastereomers or N-oxides of these compounds having the same type of activity are also provided.

Pharmaceutical compositions containing the compounds, which may also contain pharmaceutically acceptable carriers or diluents, can be used for the treatment of AIDS, asthma, arthritis, bronchitis, chronic obstructive pulmonary disease (COPD), psoriasis, allergic rhinitis, shock, atopic dermatitis, Crohn's disease, adult respiratory distress syndrome, eosinophilic granuloma, allergic conjunctivitis, osteoarthritis, ulcerative colitis and other inflammatory diseases.

The present invention encompasses a compound having the structure of Formula I,

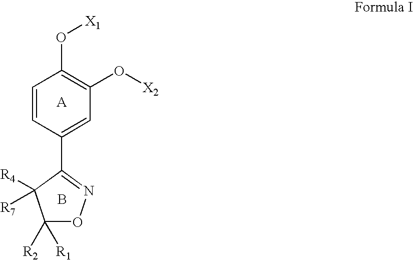

and its pharmaceutically acceptable salts, pharmaceutically acceptable solvates, enantiomers, diastereomers or N-oxides, wherein

R₁ and R₂ together forms an optionally substituted cycloalkyl or heterocyclyl ring wherein one or more optional substituent are oxo, alkyl, alkaryl, alkenyl, alkynyl, heterocyclylalkyl, cycloalkylalkyl, —SO₂NRₓR_{y}, halogen, —NH₂, —(CH₂)_{g}C(═O)NRₓR_{y}, —NHC(═O)OR₆, —NHC(═O)NRₓR_{y}, —C(═O)OR₃, —NHC(═O)Rₓ, —SO₂R₃, cyano, hydroxy, alkoxy, substituted amino, —C(═O)R₃;

R₄ can be hydrogen; alkyl; hydroxy; halogen; carboxy;

R₇ can be hydrogen; alkyl;

R₁ is independently hydrogen or alkyl and R₂ and R₄ forms an optionally substituted 4-12 membered saturated or unsaturated monocyclic or bicyclic ring system fused to ring B having 0-4 heteroatom(s) selected from the group consisting of N, O and S, wherein the substituents is one or more of oxo, alkyl, —C(═O)OR₃, —SO₂R₃, halogen, hydroxy, alkoxy, —NH₂ or substituted amino, with the proviso that R₂ and R₄ together does not form —CH₂—O—CH₂—O—CH₂—;

X₁ and X₂ can be hydrogen, alkyl, cycloalkyl, alkaryl, alkenyl, cycloalkylalkyl, heteroaryl, heterocyclyl, heteroarylalkyl, heterocyclylalkyl, —(CH₂)_{g}C(═O)NRₓR_{y} or —(CH₂)_{g1}C(═O)OR₃ (wherein g can be an integer from 0-3 and g₁ can be an integer from 1-3);

X₁ and X₂ together can optionally form a cyclic ring fused with the ring A shown in Formula I, the ring containing 3-5 carbon atoms within the ring and having 2-3 heteroatoms selected from the group consisting of N, O and S;

wherein R₃ can be alkyl, cycloalkyl or heterocyclyl;

wherein the halogen can be F, Cl, Br, or I; Rₓ, and R_{y} each independently can be hydrogen, alkyl, C₃-C₆ alkenyl, C₃-C₆ alkynyl, carboxy, cycloalkyl, —S(O)ₘR₅, aryl, alkaryl, heteroaryl, heterocyclyl, heteroarylalkyl, and heterocyclylalkyl; m can be an integer between 0-2; R₆ can be alkyl, alkenyl, alkynyl, cycloalkyl, alkaryl, heteroarylalkyl or heterocyclylalkyl;

wherein R₅ can be hydrogen, alkyl, alkenyl, alkynyl, aryl, cycloalkyl, alkaryl, heteroaryl, heteroarylalkyl, heterocyclyl or heterocyclylalkyl;

The following definitions apply to terms as used herein:

The term “alkyl,” unless otherwise specified, refers to a monoradical branched or unbranched saturated hydrocarbon having from 1 to about 20 carbon atoms. This term is exemplified by groups, such as methyl, ethyl, n-propyl, iso-propyl, n-butyl, iso-butyl, t-butyl, n-hexyl, n-decyl, tetradecyl, and the like. The alkyl groups may be further substituted with one or more substituents such as alkenyl, alkynyl, alkoxy, cycloalkyl, acyl, acylamino, acyloxy, amino, aminocarbonyl, alkoxycarbonylamino, azido, cyano, halogen, hydroxy, oxo, thiocarbonyl, carboxy, arylthio, thiol, alkylthio, aryloxy, aminosulfonyl, aminocarbonylamino, hydroxyamino, alkoxyamino, nitro, —S(O)ₙR₅ (wherein n can be 0, 1 or 2 and R₅ can be hydrogen, alkyl, alkenyl, alkynyl, aryl, cycloalkyl, alkaryl, heteroaryl, heteroarylalkyl, heterocyclyl or heterocyclylalkyl), heterocyclyl or heteroaryl. Unless otherwise constrained by the definition, all substituents may optionally be further substituted by 1-3 substituents chosen from alkyl, carboxy, aminocarbonyl, hydroxy, alkoxy, halogen, —CF₃, amino, substituted amino, cyano, and —S(O)ₙR₅ (wherein n and R₅ are the same as defined earlier) or an alkyl group as defined above that is interrupted by 1-5 atoms or groups independently chosen from oxygen, sulfur and —NRₐ— (where Rₐ can be hydrogen, alkyl, cycloalkyl, alkenyl, alkynyl, or aryl). Unless otherwise constrained by the definition, all substituents may optionally be further substituted by 1-3 substituents chosen from alkyl, carboxy, aminocarbonyl, hydroxy, alkoxy, halogen, CF₃, amino, substituted amino, cyano, and —S(O)ₙR₅ (wherein n and R₅ are the same as defined earlier); or an alkyl group as defined above that has both substituents as defined above and is also interrupted by 1-5 atoms or groups as defined above.

The term “alkenyl,” unless otherwise specified, refers to a monoradical of a branched or unbranched unsaturated hydrocarbon group preferably having from 2 to 20 carbon atoms with cis or trans geometry. Preferred alkenyl groups include ethenyl or vinyl (CH═CH₂), 1-propylene or allyl (—CH₂CH═CH₂), or iso-propylene (—C(CH₃)═CH₂), bicyclo[2.2.1]heptene, and the like. In the event that the alkenyl is attached to a heteroatom, the double bond cannot be alpha to the heteroatom. The alkenyl group may be further substituted with one or more substituents, such as alkyl, alkenyl, alkynyl, alkoxy, cycloalkyl, acyl, acylamino, acyloxy, amino, aminocarbonyl, alkoxycarbonylamino, azido, cyano, halogen, hydroxy, oxo, thiocarbonyl, carboxy, arylthio, thiol, alkylthio, aryl, aryloxy, aminosulfonyl, aminocarbonylamino, hydroxyamino, alkoxyamino, nitro, —S(O)ₙR₅ (wherein n and R₅ are the same as defined earlier), heterocyclyl or heteroaryl. Unless otherwise constrained by the definition, all substituents may be optionally further substituted by 1-3 substituents, which can be alkyl, carboxy, aminocarbonyl, hydroxy, alkoxy, halogen, —CF₃, amino, substituted amino, cyano, or —S(O)ₙR₅ (wherein R₅ and n are the same as defined earlier).

The term “alkynyl,” unless otherwise specified, refers to a monoradical of an unsaturated hydrocarbon, preferably having from 2 to 20 carbon atoms. Preferred alkynyl groups include ethynyl, (—C═CH), or propargyl (or propynyl, —CH₂C═CH), and the like. In the event that the alkynyl is attached to a heteroatom, the triple bond cannot be alpha to the heteroatom. The alkynyl group may be further substituted with one or more substituents, such as alkyl, alkenyl, alkynyl, alkoxy, cycloalkyl, acyl, acylamino, acyloxy, amino, aminocarbonyl, alkoxycarbonylamino, azido, cyano, halogen, hydroxy, oxo, thiocarbonyl, carboxy, arylthio, thiol, alkylthio, aryl, aryloxy, aminosulfonyl, aminocarbonylamino, hydroxyamino, alkoxyamino, nitro, or —S(O)ₙR₅ (wherein R₅ is the same as defined earlier). Unless otherwise constrained by the definition, all substituents may be optionally further substituted by 1-3 substituents, which can be alkyl, carboxy, aminocarbonyl, hydroxy, alkoxy, halogen, CF₃, amino, substituted amino, cyano or —S(O)ₙR₅ (wherein R₅ and n are the same as defined earlier).

The term “cycloalkyl,” unless otherwise specified, refers to saturated or unsaturated cyclic alkyl groups of from 3 to 20 carbon atoms having a single cyclic ring or multiple condensed rings, which contains an optional olefinic bond. Such cycloalkyl groups include, by way of example, single ring structures, such as cyclopropyl, cyclobutyl, cyclopentyl, cyclooctyl, cyclopropylene, cyclobutylene and the like, or multiple ring structures, such as adamantanyl, and bicyclo [2.2.1]heptane, or cyclic alkyl groups to which is fused an aryl group, for example, indane and the like. The cycloalkyl may be further substituted with one or more substituents such as alkyl, alkenyl, alkynyl, alkoxy, cycloalkyl, acyl, acylamino, acyloxy, amino, aminocarbonyl, alkoxycarbonylamino, azido, cyano, halogen, hydroxy, oxo, thiocarbonyl, carboxy, arylthio, thiol, alkylthio, aryl, aryloxy, alkaryloxy, aminosulfonyl, aminocarbonylamino, hydroxyamino, alkoxyamino, nitro, —S(O)ₙR₅ (wherein R₅ is the same as defined earlier), heteroaryl or heterocyclyl. Unless otherwise constrained by the definition, all substituents may be optionally further substituted by 1-3 substituents, which can be alkyl, carboxy, aminocarbonyl, hydroxy, alkoxy, halogen, CF₃, —NH₂, substituted amino, cyano, or —S(O)ₙR₅ (wherein R₅ and n are the same as defined earlier).

The term “alkoxy” denotes the group O-alkyl, wherein alkyl is the same as defined above.

The term “alkaryl” refers to alkyl-aryl linked through alkyl portion (wherein alkyl is the same as defined earlier) and the alkyl portion contains carbon atoms from 1-6 and aryl is same as defined below.

The term “aryl,” unless otherwise specified, refers to phenyl or naphthyl ring, and the like, optionally substituted with 1 to 3 substituents selected from the group consisting of halogen (such as F, Cl, Br, I), hydroxy, alkyl, alkenyl, alkynyl, cycloalkyl, alkoxy, aryloxy, —S(O)ₙR₅ (wherein R₅ is the same as defined earlier), cyano, nitro, carboxy, heterocyclyl, heteroaryl, heterocyclylalkyl, heteroarylalkyl, acyl and (CH₂)₀₋₃C(═O)NRₓR_{y} (wherein Rₓ and R_{y} are same as defined earlier).

The term “carboxy,” unless otherwise specified, refers to —C(═O)O—R₆, wherein R₆ can be, for example, hydrogen, alkyl, alkenyl, alkynyl, cycloalkyl, alkaryl, heteroarylalkyl or heterocyclylalkyl.

The term “heteroaryl,” unless otherwise specified, refers to an aromatic ring structure containing 5 or 6 carbon atoms, or a bicyclic aromatic group having 8 to 10 carbon atoms, with one or more heteroatom(s) independently selected from the group consisting of N, O and S, optionally substituted with 1 to 3 substituent(s), such as halogen (F, Cl, Br, I), hydroxy, alkyl, alkenyl, alkynyl, cycloalkyl, aryl, —S(O)ₙR₅ (wherein n and R₅ are the same as defined earlier), alkoxy, alkaryl, cyano, nitro, acyl or C(═O)NRₓR_{y} (wherein Rₓ and R_{y} are the same as defined earlier). Examples of heteroaryl groups include, but are not limited to, pyridinyl, pyridazinyl, pyrimidinyl, pyrrolyl, oxazolyl, thiazolyl, thienyl, isoxazolyl, triazinyl, furanyl, benzofuranyl, indolyl, benzothiazolyl, benzoxazolyl, and the like, including analogous oxygen, sulphur, and mixed hetero atom containing groups.

The term ‘heterocyclyl,” unless otherwise specified, refers to a saturated or unsaturated monocyclic or polycyclic ring having 5 to 10 atoms, in which 1 to 3 carbon atoms in a ring are replaced by heteroatoms selected from the group consisting of O, S and N, and optionally are benzofused or fused heteroaryl of 5-6 ring members and/or optionally are substituted, wherein the substituents can be halogen (F, Cl, Br, I), hydroxy, alkyl, alkenyl, alkynyl, hydroxyalkyl, cycloalkyl, carboxy, aryl, alkoxy, alkaryl, heteroaryl, heterocyclyl, heteroarylalkyl, heterocyclylalkyl, oxo, alkoxyalkyl or —S(O)ₙR₅ (wherein n and R₅ are the same as defined earlier), cyano, nitro, —NH₂ substituted amino, acyl or —C(═O)NRₓR_{y} (wherein Rₓ and R_{y} are the same as defined earlier). Examples of heterocyclyl groups include, but are not limited to, tetrahydrofuranyl, dihydrofuranyl, azabicyclohexane dihydropyridinyl, piperidinyl, isoxazoline, piperazinyl, dihydrobenzofuryl, isoindole-dione, dihydroindolyl,

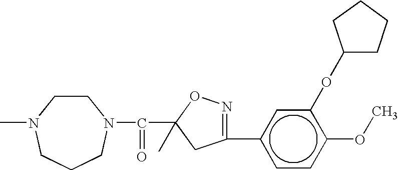

and the like.

“Heteroarylalkyl,” unless otherwise specified, refers to an alkyl-heteroaryl group, wherein the alkyl and heteroaryl portions are the same as defined earlier.

“Heterocyclylalkyl,” unless otherwise specified, refers to an alkyl-heterocyclyl group, wherein the alkyl and heterocyclyl portions of the group are the same as defined earlier.

The term “acyl” as defined herein refers to —C(═O)R″, wherein R″ is the same as defined earlier.

The term “substituted amino,” unless otherwise specified, refers to a group —N(Rₖ)₂ wherein each Rₖ can be hydrogen [provided that both Rₖ groups are not hydrogen (defined as “—NH₂”)], alkyl, alkenyl, alkynyl, alkaryl, cycloalkyl, aryl, heteroaryl, heterocyclyl, heterocyclylalkyl, heteroarylalkyl, acyl, S(O)ₘR₅ (wherein m and R₅ is the same as defined above), —C(═O)NRₓR_{y}, —C(═O)ORₓ (wherein Rₓ and R_{y} are the same as defined earlier) or —NHC(═O)NR_{y}Rₓ (wherein R_{y} and Rₓ are the same as defined earlier).

Unless otherwise constrained by the definition, all substituents optionally may be further substituted by 1-3 substituents, which can be alkyl, alkaryl, cycloalkyl, aryl, heteroaryl, heterocyclyl, carboxy, hydroxy, alkoxy, halogen, —CF₃, cyano, —C(═O)NRₓR_{y}, —O(C═O)NRₓR_{y} (wherein Rₓ and R_{y} are the same as defined earlier) and —OC(═O)NRₓR_{y} or —S(O)ₘR₅ (where R₅ is the same as defined above and m is 0, 1 or 2).

The compounds of the present invention can be used for treating AIDS, asthma, arthritis, bronchitis, chronic obstructive pulmonary disease, psoriasis, allergic rhinitis, shock, atopic dermatitis, crohn's disease, adult respiratory distress syndrome, eosinophilic granuloma, allergic conjunctivitis, osteoarthritis, ulcerative colitis and other inflammatory diseases. Accordingly, the present invention encompasses a method of treating AIDS, asthma, arthritis, bronchitis, chronic obstructive pulmonary disease, psoriasis, allergic rhinitis, shock, atopic dermatitis, crohn's disease, adult respiratory distress syndrome, eosinophilic granuloma, allergic conjunctivitis, osteoarthritis, ulcerative colitis or other inflammatory diseases, which comprises administering to a patient in need thereof a therapeutically effective amount of an isoxazoline derivative compound of the present invention, and particularly an isoxazoline derivative compound of the present invention together a pharmaceutically acceptable carrier, excipient or diluent.

In accordance with yet another aspect, there are provided processes for the preparation of the compounds as described herein.

The compounds of the present invention may be prepared by techniques well known in the art. In addition, the compounds of the present invention may be prepared following a reaction sequence as depicted below.

The compounds of this invention contain one or more asymmetric carbon atoms and thus occur as racemic mixtures, enantiomers and diastereomers. These compounds also exist as conformers/rotamers. All such isomeric forms of these compounds are expressly included in the present invention. Each stereogenic carbon may be of the R or S configuration. Although the specific compounds exemplified in this application may be depicted in a particular stereochemical configuration, compounds having either the opposite stereochemistry at any given chiral center or mixtures thereof are envisioned as part of the invention.

### DETAILED DESCRIPTION OF THE INVENTION

The compounds of the present invention may be prepared by techniques well known in the organic synthesis and familiar to a practitioner skilled in art of this invention. In addition, the process described herein may prepare the compounds of the present invention, however that may not be the only means by which the compounds described may be synthesised. Further, the various synthetic steps described herein may be performed in an alternate sequence in order to give the desired compounds.

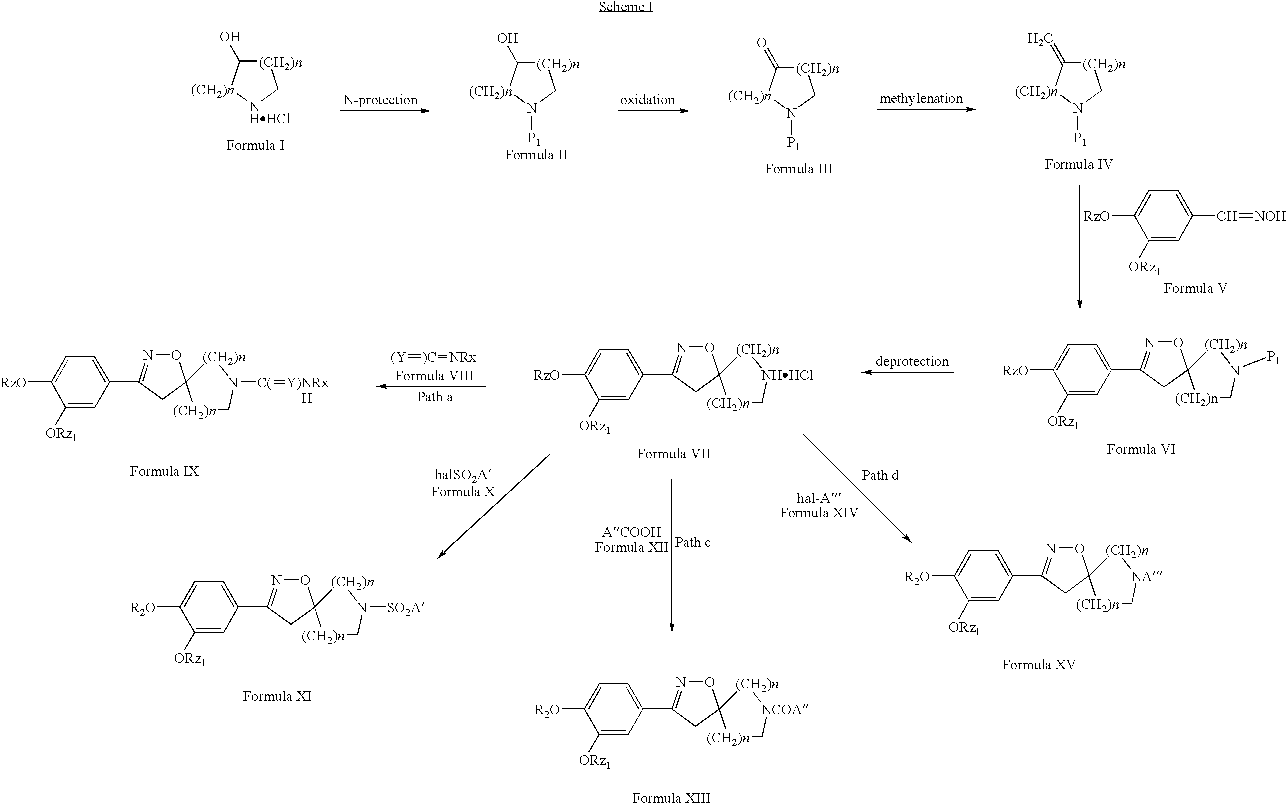

The compounds of Formulae VII, IX, XI, XIII and XV can be prepared by following the reaction sequence as depicted for example in Scheme I. Thus, a compound of Formula I (wherein n can be 1, 2 or 3) can be N-protected to give a compound of Formula II (wherein P₁ can be —C(═O)OC(CH₃)₃, —C(═O)OC(CH₃)₂CHBr₂ or —C(═O)OC(CH₃)₂CCl₃), which can be oxidized to give a compound of Formula III, which can undergo methylenation to give a compound of Formula IV, which can be reacted with a compound of Formula V (which was prepared following the procedure as described in U.S. patent application Ser. No. 10/930,569 wherein R_{z} is alkyl optionally substituted with halogen (for example, trifluoromethyl) or alkaryl (for example, benzyl) and R_{z1} can be cycloalkylalkyl, alkaryl, cycloalkyl or alkyl optionally substituted with halogen) to give a compound of Formula VI, which can be deprotected to give a compound of Formula VII, which can be reacted with

Path a: a compound of Formula VIII (wherein Y is oxygen or sulphur and Rₓ is the same as defined earlier) to give a compound of Formula IX;
Path b: a compound of Formula X (wherein A′ is —NRₓR_{y} or alkyl where Rₓ and R_{y} are the same as defined earlier) to give a compound of Formula XI;
Path c: a compound of Formula XII (wherein A″ is cycloalkyl, heterocyclyl or alkyl) to give a compound of Formula XIII; or
Path d: a compound of Formula XIV (wherein hal is Br, Cl or I and A′″ is heterocyclylalkyl, cycloalkylalkyl, alkaryl or alkyl optionally substituted with —CONRₓR_{y} wherein Rₓ and R_{y} are the same as defined earlier).

The N-protection of a compound of Formula I to give a compound of Formula II [wherein P can be —C(═O)OC(CH₃)₃] can be carried out in an organic solvent, such as, for example, dichloromethane, dichloroethane, chloroform or carbon tetrachloride, in the presence of a base, such as, for example triethylamine, diisopropylethylamine, N-methylmorpholine or pyridine.

The N-protection of a compound of Formula I to give a compound of Formula II [when P can be —C(═O)OC(CH₃)₂CHBr₂ or —C(═O)OC(CH₃)₂CCl₃] can be carried out following procedures described in Theodora W. Greene and Peter G. M. Wuts, “Protecting Groups In Organic Synthesis,” 3^{rd} edition, John Wiley and Sons, New York 1999.

The oxidation of a compound of Formula II to give a compound of Formula III can be carried out using an oxidizing agent, such as, for example, pyridinium chlorochromate, manganese dioxide, potassium permanganate or Jones reagent (CrO₃/H₂SO₄).

The methylenation of a compound of Formula III to give a compound of Formula IV can be carried out in an organic solvent, such as, for example, tetrahydrofuran, dimethylformamide, dioxane or diethylether, in the presence of a Wittig salt for example, triphenylmethylphosphonium iodide or triphenylmethylphosphonium bromide.

Alternatively, the methylenation of a compound of Formula III to give a compound of Formula IV can be carried out using Zn/CH₂Br₂/TiCl₄ in an organic solvent, such as, for example, tetrahydrofuran, dimethylformamide, dioxane or diethylether.

The reaction of a compound of Formula IV with a compound of Formula V to give a compound of Formula VI can be carried out in an organic solvent, such as, for example, dichloromethane, chloroform, carbon tetrachloride or dichloroethane, tetrahydrofuran with oxidants such as, for example, sodium hypochlorite, N-chlorosuccinimide or tert-butoxychloride in the presence of an optional base, such as, for example, pyridine, butyl lithium, N-methylmorpholine, diisopropylethylamine or triethylamine.

The deprotection of a compound of Formula VI (wherein P can be —C(═O)OC(CH₃)₃) to give a compound of Formula VII can be carried out in an organic solvent, such as, for example, methanol, ethanol, propanol or isopropylalcohol, in the presence of an alcoholic acid solution, such as, for example, ethanolic hydrochloric acid or methanolic hydrochloric acid.

The deprotection of a compound of Formula VI (wherein P can be —C(═O)OC(CH₃)₂CHBr₂) can be carried out in an organic solvent, such as, for example, ethanol, methanol, propanol or isopropylalcohol in the presence of hydrobromic acid or hydrochloric acid).

The deprotection of a compound of Formula VI (wherein P can be —C(═O)OC(CH₃)₂CCl₃) can be carried out by a supernucleophile, such as, for example, lithium cobalt (I) phthalocyanine, zinc and acetic acid or cobalt phthalocyanine.

The compound of Formula VII can be reacted with a compound of Formula VIII (path a) to give a compound of Formula IX in an organic solvent, such as, for example, dichloroethane, dichloromethane, chloroform or carbon tetrachloride in the presence of a base such as, for example, triethylamine, diisopropylethylamine, N-methylmorpholine or pyridine.

The compound of Formula VII can be reacted with a compound of Formula X (path b) to give a compound of Formula XI in an organic solvent, such as, for example, dichloroethane, dichloromethane, chloroform or carbon tetrachloride in the presence of a base such as, for example, triethylamine, diisopropylethylamine, N-methylmorpholine or pyridine.

The compound of Formula VII can be reacted with a compound of Formula XII (path c) to give a compound of Formula XIII in an organic solvent, such as, for example, dimethylformamide, tetrahydrofuran, diethylether or dioxane in the presence of a base such as, for example, N-methylmorpholine, triethylamine, diisopropylethylamine or pyridine.

The compound of Formula VII can be reacted with a compound of Formula XIV (path d) to give a compound of Formula XV in an organic solvent, such as, for example, dimethylformamide, tetrahydrofuran, diethylether or dioxane in the presence of a base such as, for example, potassium carbonate, sodium carbonate or lithium carbonate.

Some representative compounds which can be prepared following Scheme I include:
Tert-butyl 3-[3-(cyclopentyloxy)-4-methoxyphenyl]-1-oxa-2,7-diazaspiro[4.5]dec-2-ene-7-carboxylate (Compound No. 21),
Hydrochloride salt of 3-[3-(cyclopentyloxy)-4-methoxyphenyl]-1-oxa-2,7-diazaspiro[4.5]dec-2-ene (Compound No. 25),
   Some representative compounds which can be prepared following Scheme I, path a include:
3-[3-(Cyclopentyloxy)-4-methoxyphenyl]-N-(4-fluorophenyl)-1-oxa-2,7-diazaspiro[4.4]non-2-ene-7-carboxamide (Compound No. 2),
N-butyl-3-[3-(cyclopentyloxy)-4-methoxyphenyl]-1-oxa-2,7-diazaspiro[4.4]non-2-ene-7-carboxamide (Compound No. 5),
N-butyl-3-[3-(cyclopentyloxy)-4-methoxyphenyl]-1-oxa-2,8-diazaspiro[4.5]dec-2-ene-8-carboxamide (Compound No. 9),
N-benzyl-3-[3-(cyclopentyloxy)-4-methoxyphenyl]-1-oxa-2,7-diazaspiro[4.4]non-2-ene-7-carboxamide (Compound No. 19),
N-Benzyl-3-[3-(cyclopentyloxy)-4-methoxyphenyl]-1-oxa-2,8-diazaspiro[4.5]dec-2-ene-8-carboxamide (Compound No. 32),
3-[3-(Cyclopentyloxy)-4-methoxyphenyl]-1-oxa-2,8-diazaspiro[4.5]dec-2-ene-8-carboxamide (Compound No. 143),
N-Butyl-3-[3-(cyclopentyloxy)-4-methoxyphenyl]-1-oxa-2,7-diazaspiro[4.5]dec-2-ene-7-carboxamide (Compound No. 144).
   Some representative compounds which can be prepared following Scheme I, path b include:
3-[3-(cyclopentyloxy)-4-methoxyphenyl]-N,N-dimethyl-1-oxa-2,7-diazaspiro[4.4]non-2-ene-7-sulfonamide (Compound No. 4),
3-[3-(cyclopentyloxy)-4-methoxyphenyl]-8-(methylsulfonyl)-1-oxa-2,8-diazaspiro[4.5]dec-2-ene (Compound No. 10), 3-[3-(Cyclopentyloxy)-4-methoxyphenyl]-7-(methylsulfonyl)-1-oxa-2,7-diazaspiro[4.5]dec-2-ene (Compound No. 145).
   Some representative compounds which can be prepared following Scheme I, path c include:
3-[3-(Cyclopentyloxy)-4-methoxyphenyl]-7-(tetrahydrofuran-3-ylcarbonyl)-1-oxa-2,7-diazaspiro[4.4]non-2-ene (Compound No. 3),
Hydrochloride salt of 3-[3-(cyclopentyloxy)-4-methoxyphenyl]-8-prolyl-1-oxa-2,8-diazaspiro[4.5]dec-2-ene (Compound No. 7),
3-[3-(cyclopentyloxy)-4-methoxyphenyl]-7-(cyclopropylcarbonyl)-1-oxa-2,7-diazaspiro[4.4]non-2-ene (Compound No. 18),
7-acetyl-3-[3-(cyclopentyloxy)-4-methoxyphenyl]-1-oxa-2,7-diazaspiro[4.4]non-2-ene (Compound No. 20),
8-Acetyl-3-[3-(cyclopentyloxy)-4-methoxyphenyl]-1-oxa-2,8-diazaspiro[4.5]dec-2-ene (Compound No. 48),
8-(Cyclopentylcarbonyl)-3-[3-(cyclopentyloxy)-4-methoxyphenyl]-1-oxa-2,8-diazaspiro[4.5]dec-2-ene (Compound No. 49),
7-(Cyclopentylcarbonyl)-3-[3-(cyclopentyloxy)-4-methoxyphenyl]-1-oxa-2,7-diazaspiro[4.5]dec-2-ene (Compound No. 141),
7-Acetyl-3-[3-(cyclopentyloxy)-4-methoxyphenyl]-1-oxa-2,7-diazaspiro[4.5]dec-2-ene (Compound No. 155).
   Some representative compounds which can be prepared following Scheme I, path d include:
2-{3-[3-(Cyclopentyloxy)-4-methoxyphenyl]-1-oxa-2,7-diazaspiro[4.4]non-2-en-7-yl}acetamide (Compound No. 6),
3-[3-(Cyclopentyloxy)-4-methoxyphenyl]-8-(2-morpholin-4-yl-ethyl)-1-oxa-2,8-diazaspiro[4.5]dec-2-ene (Compound No. 8),
3-[3-(Cyclopentyloxy)-4-methoxyphenyl]-7-isopropyl-1-oxa-2,7-diazaspiro[4.4]non-2-ene (Compound No. 17),
3-[3-(Cyclopentyloxy)-4-methoxyphenyl]-8-(cyclopropylmethyl)-1-oxa-2,8-diazaspiro[4.5]dec-2-ene (Compound No. 31),
8-Benzyl-3-[3-(cyclopentyloxy)-4-methoxyphenyl]-1-oxa-2,8-diazaspiro[4.5]dec-2-ene (Compound No. 38),
3-[3-(Cyclopentyloxy)-4-methoxyphenyl]-8-(2-piperidin-1-ylethyl)-1-oxa-2,8-diazaspiro[4.5]dec-2-ene (Compound No. 50),
3-[3-(Cyclopentyloxy)-4-methoxyphenyl]-8-ethyl-1-oxa-2,8-diazaspiro[4.5]dec-2-ene (Compound No. 54).

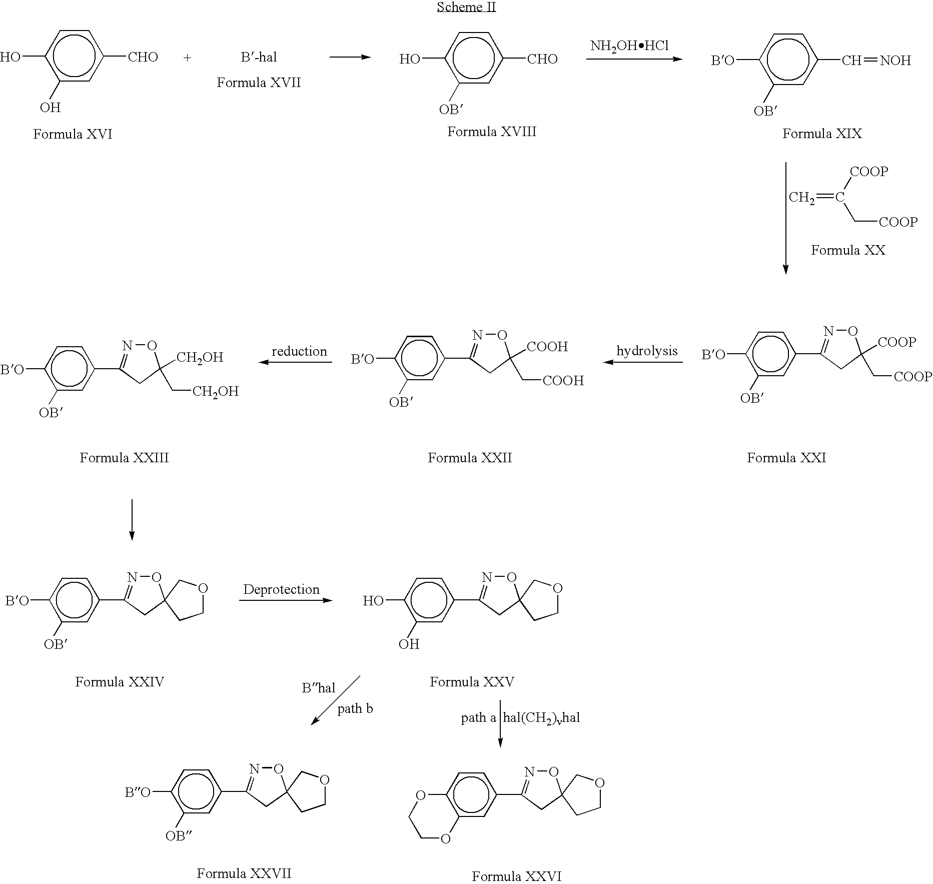

Compounds of Formulae XXIV, XXV, XXVI and XXVII can be prepared, for example, by following a reaction sequence of Scheme II. Thus, the compound of Formula XVI can be reacted with a compound of Formula XVII (wherein B′ can be alkaryl) to give a compound of Formula XVIII, which can be reacted with hydroxylamine hydrochloride to give a compound of Formula XIX, which can be reacted with a compound of Formula XX (wherein P can be alkyl or alkaryl) to give a compound of Formula XXI, which can undergo hydrolysis to give a compound of Formula XXII, which can undergo reduction to give a compound of Formula XXIII, which can undergo ring cyclisation to give a compound of Formula XXIV which can undergo deprotection to give a compound of Formula XXV, which can be reacted with

Path a: a compound of Formula hal(CH₂)ᵥhal [wherein hal is (Br, Cl or I) and v is an integer from 1-4] to give a compound of Formula XXVI; or

Path b: a compound of Formula B″ hal (wherein B″ is alkyl) and hal is the same as defined above) to give a compound of Formula XXVII.

The reaction of compound of Formula XVI with a compound of Formula XVII to give a compound of Formula XVIII can be carried out in an organic solvent, such as, for example, dimethylformamide, tetrahydrofuran, diethylether or dioxane, in the presence of base, such as, for example, potassium carbonate, sodium carbonate or sodium bicarbonate.

The reaction of a compound of Formula XVIII with hydroxylamine hydrochloride to give a compound of Formula XIX can be carried out in an organic solvent, such as, for example, ethanol, methanol, propanol or isopropylalcohol.

The compound of Formula XIX can be reacted with a compound of Formula XX to give a compound of Formula XXI in an organic solvent, such as, for example, dichloromethane, chloroform, carbon tetrachloride or dichloroethane with oxidants such as, for example, sodium hypochlorite, N-chlorosuccinimide or tert-butoxychloride in the presence of an optional base, such as, for example, pyridine, butyl lithium, N-methylmorpholine, diisopropylethylamine or triethylamine

The hydrolysis of a compound of Formula XXI to give a compound of Formula XXII can be carried out in a solvent system, such as, for example, tetrahydrofuran, methanol, dioxane or ethanol, in water in the presence of base, such as, for example, lithium hydroxide, sodium hydroxide or potassium hydroxide.

The compound of Formula XXII can undergo reduction to give a compound of Formula XXIII in an organic solvent, such as, for example, tetrahydrofuran, dimethylformamide, dioxane or diethyl ether, with reducing agent, such as, for example, sodium borohydride or lithium borohydride or lithium aluminium hydride.

The compound of Formula XXIII can undergo ring cyclisation to give a compound of Formula XXIV in an organic solvent, such as, for example, tetrahydrofuran, dimethylformamide, dioxane or diethyl ether in the presence of a redox couple. The oxidizing part of the redox couple is selected from the group diisopropylazodicarboxylate (DIAD), diethylazodicarboxylate (DEAD), N,N,N′,N′-tetramethylazodicarboxylate (TMAD), 1,1′-(azodicarbonyl) dipiperidine (ADDP), cyanomethylenetributylphosphorane (CMBP), 4,7-dimethyl-3,5,7-hexahydro-1,2,4,7-tetrazocin-3,8-dione (DHTD) or N,N,N′,N,′-tetraisopropylazodicarboxamide (TIPA). The reduction part of the redox couple is phosphine such as, for example, trialkylphosphine (such as tributylphosphine), triarylphosphine (such as triphenylphosphine), tricycloalkylphosphine (such as triscyclohexylphosphine) or tetraheteroarylphosphine. The phosphine reagents with a combination of aryl, alkyl or heteroaryl substituents may also be used (such as diphenylpyridylphosphine).

The compound of Formula XXIV can be deprotected to give a compound of Formula XXV in an organic solvent, such as, for example, methanol, ethanol, propanol or isopropylalcohol with a deprotecting agent, such as, for example, palladium on carbon or palladium on carbon with ammonium formate.

The compound of Formula XXV (path a) can be reacted with a compound of Formula hal(CH₂)ᵥhal to give a compound of Formula XXVI in an organic solvent such as, for example, dimethylformamide, tetrahydrofuran, diethyl ether or dioxane in the presence of a base such as, for example, potassium carbonate, sodium carbonate or lithium carbonate.

The compound of Formula XXV (path b) can be reacted with a compound of Formula B″ hal to give a compound of Formula XXVII in an organic solvent such as, for example, dimethylformamide, tetrahydrofuran, diethylether or dioxane in the presence of a base such as, for example, potassium carbonate, sodium carbonate or lithium carbonate.

Some representative compounds which may be prepared following Scheme II include:
3-[3,4-Bis(benzyloxy)phenyl]-1,7-dioxa-2-azaspiro[4.4]non-2-ene (Compound No. 33), 4-(1,7-Dioxa-2-azaspiro[4.4]non-2-en-3-yl)benzene-1,2-diol (Compound No. 34).
   Some representative compounds which may be prepared following Scheme II, path a include:
3-(2,3-Dihydro-1,4-benzodioxin-6-yl)-1,7-dioxa-2-azaspiro[4.4]non-2-ene (Compound No. 51).
   Some representative compounds prepared following Scheme II, path b include:
3-[3,4-bis(2-morpholin-4-ylethoxy)phenyl]-1,7-dioxa-2-azaspiro[4.4]non-2-ene (Compound No. 12),
3-(3,4-diisopropoxyphenyl)-1,7-dioxa-2-azaspiro[4.4]non-2-ene (Compound No. 13),
3-[3,4-bis(cyclopentyloxy)phenyl]-1,7-dioxa-2-azaspiro[4.4]non-2-ene (Compound No. 27),
3-[3,4-Bis(cyclopropylmethoxy)phenyl]-1,7-dioxa-2-azaspiro[4.4]non-2-ene (Compound No. 28),

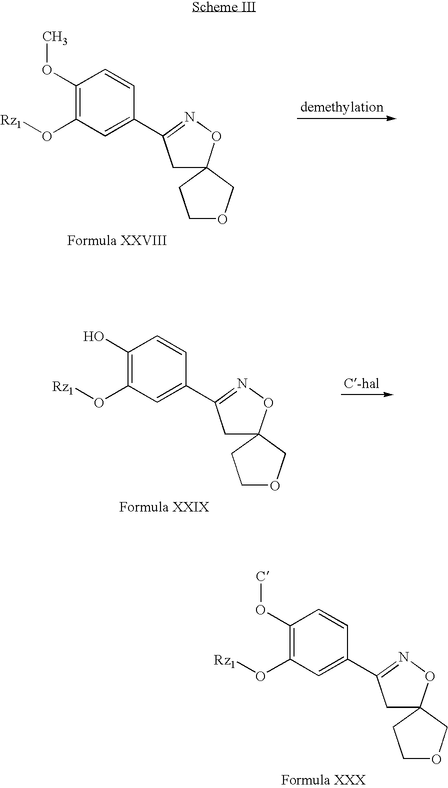

The compounds of Formula XXX can be prepared by following the procedure as depicted in scheme III. Thus a compound of Formula XXVIII (wherein Rz₁ is the same as defined earlier) undergoes demethylation to give a compound of Formula XXIX, which was reacted, with a compound of Formula C′-hal (wherein C′ is heterocyclylalkyl, cycloalkylalkyl, cycloalkyl or C₂₋₁₀ alkyl optionally substituted with halogen) to give a compound of Formula XXX.

The demethylation of a compound of Formula XXVIII to give a compound of Formula XXIX can be carried out with reducing agent such as, for example, sodium ethane thiolate, sodium decane thiolate, sodium dodecane thiolate, sodium thiocresolate in the presence of solvent for example N,N-dimethylacetamide, hexamethyl phosphoramide or dimethylformamide.

The reaction of a compound of Formula XXIX with a compound of Formula C′-hal can be carried out in an organic solvent such as, for example, dimethylformamide, tetrahydrofuran, diethyl ether or dioxane in the presence of a base such as, for example, potassium carbonate, sodium carbonate or lithium carbonate.

Some representative compounds which may be prepared following Scheme III include:
2-(Cyclopentyloxy)-4-(1,7-dioxa-2-azaspiro[4.4]non-2-en-3-yl)phenol (Compound No. 62),
3-(4-Butoxy-3-isobutoxyphenyl)-1,7-dioxa-2-azaspiro[4.4]non-2-ene (Compound No. 63),
3-(3-Isobutoxy-4-propoxyphenyl)-1,7-dioxa-2-azaspiro[4.4]non-2-ene (Compound No. 64),
3-[3-Butoxy-4-(cyclopropylmethoxy)phenyl]-1,7-dioxa-2-azaspiro[4.4]non-2-ene (Compound No. 65),
3-(3-Butoxy-4-ethoxyphenyl)-1,7-dioxa-2-azaspiro[4.4]non-2-ene (Compound No. 66),
3-[3-Butoxy-4-(cyclohexyloxy)phenyl]-1,7-dioxa-2-azaspiro[4.4]non-2-ene (Compound No. 67),
3-[3-(Cyclohexylmethoxy)-4-ethoxyphenyl]-1,7-dioxa-2-azaspiro[4.4]non-2-ene (Compound No. 68),
3-[3-(Cyclohexylmethoxy)-4-isopropoxyphenyl]-1,7-dioxa-2-azaspiro[4.4]non-2-ene (Compound No. 69),
3-[4-Butoxy-3-(cyclohexylmethoxy)phenyl]-1,7-dioxa-2-azaspiro[4.4]non-2-ene (Compound No. 70),
3-(4-Isobutoxy-3-isopropoxyphenyl)-1,7-dioxa-2-azaspiro[4.4]non-2-ene (Compound No. 71),
3-(4-Butoxy-3-isopropoxyphenyl)-1,7-dioxa-2-azaspiro[4.4]non-2-ene (Compound No. 72),
3-[4-(Cyclohexylmethoxy)-3-isopropoxyphenyl]-1,7-dioxa-2-azaspiro[4.4]non-2-ene (Compound No. 73),
3-[3-Isopropoxy-4-(2-morpholin-4-ylethoxy)phenyl]-1,7-dioxa-2-azaspiro[4.4]non-2-ene (Compound No. 74),
3-[3-(Cyclopropylmethoxy)-4-isopropoxyphenyl]-1,7-dioxa-2-azaspiro[4.4]non-2-ene (Compound No. 75),
3-[3-(Cyclopropylmethoxy)-4-(2-morpholin-4-ylethoxy)phenyl]-1,7-dioxa-2-azaspiro[4.4]non-2-ene (Compound No. 76),
3-[4-Butoxy-3-(cyclopropylmethoxy)phenyl]-1,7-dioxa-2-azaspiro[4.4]non-2-ene (Compound No. 77),
3-[3-(Cyclopropylmethoxy)-4-isopropoxyphenyl]-1,7-dioxa-2-azaspiro[4.4]non-2-ene (Compound No. 78),
3-(3-Isobutoxy-4-isopropoxyphenyl)-1,7-dioxa-2-azaspiro[4.4]non-2-ene (Compound No. 79),
3-[4-(Cyclopropylmethoxy)-3-isobutoxyphenyl]-1,7-dioxa-2-azaspiro[4.4]non-2-ene (Compound No. 80),
3-[4-(cyclohexyloxy)-3-(cyclopentyloxy)phenyl]-1,7-dioxa-2-azaspiro[4.4]non-2-ene (Compound No. 81)
3-[4-(Cyclohexylmethoxy)-3-(cyclopentyloxy)phenyl]-1,7-dioxa-2-azaspiro[4.4]non-2-ene (Compound No. 82),
3-[4-(Cyclopropylmethoxy)-3-(cyclopentyloxy)phenyl]-1,7-dioxa-2-azaspiro[4.4]non-2-ene (Compound No. 83),
3-[3-(Cyclopentyloxy)-4-isobutoxyphenyl]-1,7-dioxa-2-azaspiro[4.4]non-2-ene (Compound No. 84),
3-[3-(Cyclopentyloxy)-4-ethoxyphenyl]-1,7-dioxa-2-azaspiro[4.4]non-2-ene (Compound No. 85),
3-[3-(Cyclopropylmethoxy)-4-ethoxyphenyl]-1,7-dioxa-2-azaspiro[4.4]non-2-ene (Compound No. 86),
3-[4-(Cyclopentyloxy)-3-isobutoxyphenyl]-1,7-dioxa-2-azaspiro[4.4]non-2-ene (Compound No. 87),
3-[3-Isopropoxy-4-(2-morpholin-4-ylethoxy)phenyl]-1,7-dioxa-2-azaspiro[4.4]non-2-ene (Compound No. 88),
3-(4-Ethoxy-3-isobutoxyphenyl)-1,7-dioxa-2-azaspiro[4.4]non-2-ene (Compound No. 89)
3-[3-(Cyclopentyloxy)-4-propoxyphenyl]-1,7-dioxa-2-azaspiro[4.4]non-2-ene (Compound No. 90),
3-[4-Butoxy-3-(cyclopentyloxy)phenyl]-1,7-dioxa-2-azaspiro[4.4]non-2-ene (Compound No. 91),
3-[3-(Cyclopentyloxy)-4-isopropoxyphenyl]-1,7-dioxa-2-azaspiro[4.4]non-2-ene (Compound No. 92),
3-[3-(Cyclopentyloxy)-4-(cycloheptyloxy)phenyl]-1,7-dioxa-2-azaspiro[4.4]non-2-ene (Compound No. 93),
3-[3-(Cyclopentyloxy)-4-(2-morpholin-4-ylethoxy)phenyl]-1,7-dioxa-2-azaspiro[4.4]non-2-ene (Compound No. 94),
3-[4-(Cyclohexylmethoxy)-3-isobutoxyphenyl]-1,7-dioxa-2-azaspiro[4.4]non-2-ene (Compound No. 95),
3-[4-(Cyclohexylmethoxy)-3-(cyclopropylmethoxy)phenyl]-1,7-dioxa-2-azaspiro[4.4]non-2-ene (Compound No. 96),
3-[3-(Cyclopropylmethoxy)-4-propoxyphenyl]-1,7-dioxa-2-azaspiro[4.4]non-2-ene (Compound No. 97),
3-[4-(Cyclopentyloxy)-3-(cyclopropylmethoxy)phenyl]-1,7-dioxa-2-azaspiro[4.4]non-2-ene (Compound No. 98),
3-[4-(Cyclopropylmethoxy)-3-isopropoxyphenyl]-1,7-dioxa-2-azaspiro[4.4]non-2-ene (Compound No. 99),
3-[4-(Cyclopentyloxy)-3-isopropoxyphenyl]-1,7-dioxa-2-azaspiro[4.4]non-2-ene (Compound No. 100),
3-(3-Isopropoxy-4-propoxyphenyl)-1,7-dioxa-2-azaspiro[4.4]non-2-ene (Compound No. 101),
3-(4-Ethoxy-3-isopropoxyphenyl)-1,7-dioxa-2-azaspiro[4.4]non-2-ene (Compound No. 102),
3-[3-Butoxy-4-(2-morpholin-4-ylethoxy)phenyl]-1,7-dioxa-2-azaspiro[4.4]non-2-ene (Compound No. 103),
3-[3-Butoxy-4-(cyclopentyloxy)phenyl]-1,7-dioxa-2-azaspiro[4.4]non-2-ene (Compound No. 104),
3-(3-Butoxy-4-propoxyphenyl)-1,7-dioxa-2-azaspiro[4.4]non-2-ene (Compound No. 105),
3-(3-Butoxy-4-isopropoxyphenyl)-1,7-dioxa-2-azaspiro[4.4]non-2-ene (Compound No. 106),
3-[3-(Cyclohexylmethoxy)-4-propoxyphenyl]-1,7-dioxa-2-azaspiro[4.4]non-2-ene (Compound No. 107),
3-[3-(Cyclohexylmethoxy)-4-isobutoxyphenyl]-1,7-dioxa-2-azaspiro[4.4]non-2-ene (Compound No. 108),
3-[3-(Cyclohexylmethoxy)-4-(cyclopentyloxy)phenyl]-1,7-dioxa-2-azaspiro[4.4]non-2-ene (Compound No. 109),
3-[3-(Cyclohexylmethoxy)-4-(cyclopropylmethoxy)phenyl]-1,7-dioxa-2-azaspiro[4.4]non-2-ene (Compound No. 110),
3-[4-(Cyclohexylmethoxy)-3-propoxyphenyl]-1,7-dioxa-2-azaspiro[4.4]non-2-ene (Compound No. 111),
3-[4-(Cyclopropylmethoxy)-3-propoxyphenyl]-1,7-dioxa-2-azaspiro[4.4]non-2-ene (Compound No. 112),
3-[4-(Cyclopentyloxy)-3-propoxyphenyl]-1,7-dioxa-2-azaspiro[4.4]non-2-ene (Compound No. 113),
3-[4-(3-Isobutoxy)-3-propoxyphenyl]-1,7-dioxa-2-azaspiro[4.4]non-2-ene (Compound No. 114),
3-[3-(Cycloheptyloxy)-4-(cyclopropylmethoxy)phenyl]-1,7-dioxa-2-azaspiro[4.4]non-2-ene (Compound No. 115),
3-[3-(Cycloheptyloxy)-4-propoxyphenyl]-1,7-dioxa-2-azaspiro[4.4]non-2-ene (Compound No. 116),
3-[4-Butoxy-3-(cycloheptyloxy)phenyl]-1,7-dioxa-2-azaspiro[4.4]non-2-ene (Compound No. 117),
3-[3-(Cycloheptyloxy)-4-isopropoxyphenyl]-1,7-dioxa-2-azaspiro[4.4]non-2-ene (Compound No. 118),
3-[3-(Cycloheptyloxy)-4-(cyclopentyloxy)phenyl]-1,7-dioxa-2-azaspiro[4.4]non-2-ene (Compound No. 119),
3-(3-Ethoxy-4-propoxyphenyl)-1,7-dioxa-2-azaspiro[4.4]non-2-ene (Compound No. 120),
3-[4-(Cycloheptyloxy)-3-ethoxyphenyl]-1,7-dioxa-2-azaspiro[4.4]non-2-ene (Compound No. 121),
3-[4-(Cyclopropylmethoxy)-3-ethoxyphenyl]-1,7-dioxa-2-azaspiro[4.4]non-2-ene (Compound No. 122),
3-[4-(Cyclohexylmethoxy)-3-ethoxyphenyl]-1,7-dioxa-2-azaspiro[4.4]non-2-ene (Compound No. 123),
3-(3-Butoxy-4-isobutoxyphenyl)-1,7-dioxa-2-azaspiro[4.4]non-2-ene (Compound No. 125),
3-(3-Ethoxy-4-isopropoxyphenyl)-1,7-dioxa-2-azaspiro[4.4]non-2-ene (Compound No. 126),
3-[4-(Cyclopentyloxy)-3-ethoxyphenyl]-1,7-dioxa-2-azaspiro[4.4]non-2-ene (Compound No. 127),
3-(4-Butoxy-3-ethoxyphenyl)-1,7-dioxa-2-azaspiro[4.4]non-2-ene (Compound No. 128),
3-(3-Ethoxy-4-isobutoxyphenyl)-1,7-dioxa-2-azaspiro[4.4]non-2-ene (Compound No. 129),
3-[3-(Cycloheptyloxy)-4-isobutoxyphenyl]-1,7-dioxa-2-azaspiro[4.4]non-2-ene (Compound No. 130),
3-[3-(Cycloheptyloxy)-4-(cyclopentyloxy)phenyl]-1,7-dioxa-2-azaspiro[4.4]non-2-ene (Compound No. 131),
3-[3-(Cycloheptyloxy)-4-ethoxyphenyl]-1,7-dioxa-2-azaspiro[4.4]non-2-ene (Compound No. 132),
3-(4-Butoxy-3-propoxyphenyl)-1,7-dioxa-2-azaspiro[4.4]non-2-ene (Compound No. 133),
3-(4-Ethoxy-3-propoxyphenyl)-1,7-dioxa-2-azaspiro[4.4]non-2-ene (Compound No. 134),
3-[4-(Morpholin-4-ylethoxy)-3-propoxyphenyl]-1,7-dioxa-2-azaspiro[4.4]non-2-ene (Compound No. 135),
3-(4-Isopropoxy-3-propoxyphenyl)-1,7-dioxa-2-azaspiro[4.4]non-2-ene (Compound No. 136),
3-[4-(Difluoromethoxy)-3-(2,3-dihydro-1H-inden-2-yloxy)phenyl]-1,7-dioxa-2-azaspiro[4.4]non-2-ene (Compound No. 151),
3-[4-(Cyclopentyloxy)-3-(2,3-dihydro-1H-inden-2-yloxy)phenyl]-1,7-dioxa-2-azaspiro[4.4]non-2-ene (Compound No. 152),
3-[4-Butoxy-3-(2,3-dihydro-1H-inden-2-yloxy)phenyl]-1,7-dioxa-2-azaspiro[4.4]non-2-ene (Compound No. 153),
3-[3-(2,3-Dihydro-1H-inden-2-yloxy)-4-ethoxyphenyl]-1,7-dioxa-2-azaspiro[4.4]non-2-ene (Compound No. 157),
3-[3-(2,3-Dihydro-1H-inden-2-yloxy)-4-propoxyphenyl]-1,7-dioxa-2-azaspiro[4.4]non-2-ene (Compound No. 158),
3-[4-(Cyclopropylmethoxy)-3-(2,3-dihydro-1H-inden-2-yloxy)phenyl]-1,7-dioxa-2-azaspiro[4.4]non-2-ene (Compound No. 159),
3-[3-(2,3-Dihydro-1H-inden-2-yloxy)-4-isopropoxyphenyl]-1,7-dioxa-2-azaspiro[4.4]non-2-ene (Compound No. 160),
2-(2,3-Dihydro-1H-inden-2-yloxy)-4-(1,7-dioxa-2-azaspiro[4.4]non-2-en-3-yl)phenol (Compound No. 161),

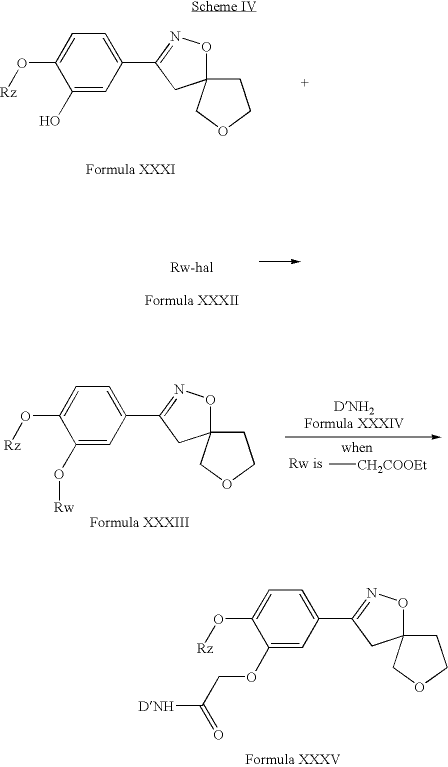

Compounds of Formulae XXXIII and XXXV can be prepared, for example, by following the reaction sequence as depicted, for example, in Scheme IV. Thus, the compound of Formula XXXI (prepared following the procedure reported in U.S. patent application Ser. No. 10/930,569 wherein Rz is the same as defined above) can be reacted with a compound of Formula XXXII [wherein R_{w} can be heteroarylalkyl, alkenyl or alkyl optionally substituted with cyano, carboxy or halogen and hal can be Br, Cl or I) to give a compound of Formula XXXIII, which can be reacted with a compound of formula XXXIV (wherein D′ is cycloalkyl or hydrogen) to give a compound of Formula XXXV.

The reaction of a compound of Formula XXXI with a compound of Formula XXXII to give a compound of Formula XXXIII can be carried out in an organic solvent, such as, for example, dimethylformamide, tetrahydrofuran, diethylether or dioxane, in the presence of base, such as, for example, potassium carbonate, sodium carbonate or sodium bicarbonate.

The compound of Formula XXXIII can be reacted with a compound of Formula XXXIV to give a compound of Formula XXXV.

Particular compounds which can be formed following the procedure shown in Scheme VII include:
3-[3-(Difluoromethoxy)-4-methoxyphenyl]-1,7-dioxa-2-azaspiro[4.4]non-2-ene (Compound No. 40),
3-[3-(Allyloxy)-4-methoxyphenyl]-1,7-dioxa-2-azaspiro[4.4]non-2-ene (Compound No. 60),
3-[3-(2-Chloroethoxy)-4-methoxyphenyl]-1,7-dioxa-2-azaspiro[4.4]non-2-ene (Compound No. 61),
3-[4-Methoxy-3-(pyridin-3-ylmethoxy)phenyl]-1,7-dioxa-2-azaspiro[4.4]non-2-ene (Compound No. 146),
3-[4-Methoxy-3-(pyridin-2-ylmethoxy)phenyl]-1,7-dioxa-2-azaspiro[4.4]non-2-ene (Compound No. 156),
N-cyclopropyl-2-[5-(1,7-dioxa-2-azaspiro[4.4]non-2-en-3-yl)-2-methoxyphenoxy]acetamide (Compound No. 162),
2-[5-(1,7-Dioxa-2-azaspiro[4.4]non-2-en-3-yl)-2-methoxyphenoxy]acetamide (Compound No. 164),
Ethyl [5-(1,7-dioxa-2-azaspiro[4.4]non-2-en-3-yl)-2-methoxyphenoxy]acetate (Compound No. 165),
[5-(1,7-Dioxa-2-azaspiro[4.4]non-2-en-3-yl)-2-methoxyphenoxy]acetonitrile (Compound No. 166),

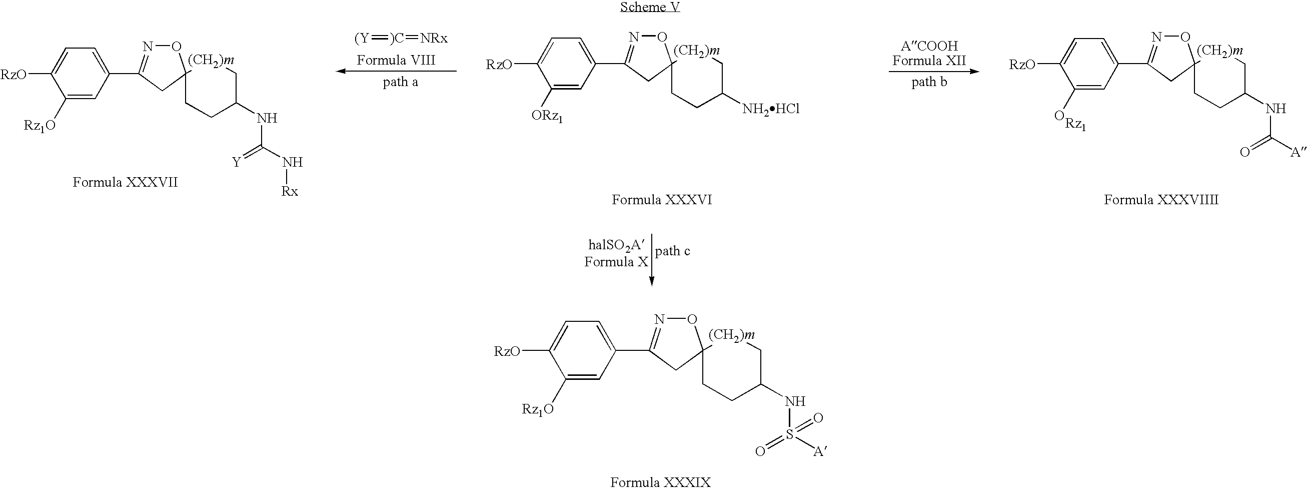

The compounds of Formulae XXXVII, XXXVIII and XXXIX can be prepared by following the procedure as depicted in Scheme V. Thus a compound of Formula XXXVI (prepared following the procedure disclosed in U.S. patent application Ser. No. 10/930,569 wherein Rz and Rz1 are the same as defined earlier) can be reacted with

Path a: a compound of Formula VIII (wherein Y and Rₓ are the same as defined earlier) to give a compound of Formula XXXVII;
Path b: a compound of Formula XII (wherein A″ is the same as defined earlier) to give a compound of Formula XXXVIII; or
Path c: a compound of Formula X (wherein A′ is the same as defined earlier) to give a compound of Formula XXXIX.

The compound of Formula XXXVI can be reacted with a compound of Formula VIII (path a) to give a compound of Formula XXXVII in an organic solvent, such as, for example, dichloroethane, dichloromethane, chloroform or carbon tetrachloride in the presence of a base such as, for example, triethylamine, diisopropylethylamine, N-methylmorpholine or pyridine.

The compound of Formula XXXVI can be reacted with a compound of Formula XII (path b) to give a compound of Formula XXXVIII in an organic solvent, such as, for example, dimethylformamide, tetrahydrofuran, diethylether or dioxane in the presence of a base such as, for example, N-methylmorpholine, triethylamine, diisopropylethylamine or pyridine.

The compound of Formula XXXVI can be reacted with a compound of Formula X (path c) to give a compound of Formula XXXIX in an organic solvent, such as, for example, dichloroethane, dichloromethane, chloroform or carbon tetrachloride in the presence of a base such as, for example, triethylamine, diisopropylethylamine, N-methylmorpholine or pyridine.

Some representative compounds which may be prepared following Scheme V, path a include:
N-butyl-N′-{3-[3-(cyclopentyloxy)-4-methoxyphenyl]-1-oxa-2-azaspiro[4.5]dec-2-en-8-yl}urea (Compound No. 22),
N-{3-[3-(cyclopentyloxy)-4-methoxyphenyl]-1-oxa-2-azaspiro[4.5]dec-2-en-8-yl}-N′-(2-methoxyphenyl)urea (Compound No. 23),
Tert-butyl [({3-[3-(cyclopentyloxy)-4-methoxyphenyl]-1-oxa-2-azaspiro[4.5]dec-2-en-8-yl}amino)carbonyl]carbamate (Compound No. 46),
   Some representative compounds which may be prepared following Scheme V, path b include:
N-{3-[3-(Cyclopentyloxy)-4-methoxyphenyl]-1-oxa-2-azaspiro[4.5]dec-2-en-8-yl}cyclopentanecarboxamide (Compound No. 47),
N-{3-[3-(Cyclopentyloxy)-4-methoxyphenyl]-1-oxa-2-azaspiro[4.5]dec-2-en-8-yl}-2-fluorobenzamide (Compound No. 138),
N-{3-[3-(cyclopentyloxy)-4-methoxyphenyl]-1-oxa-2-azaspiro[4.5]dec-2-en-8-yl}benzamide (Compound No. 139).
   Some representative compounds which may be prepared following Scheme V, path c include:
N-{3-[3-(Cyclopentyloxy)-4-methoxyphenyl]-1-oxa-2-azaspiro[4.5]dec-2-en-8-yl}methanesulfonamide (Compound No. 58),

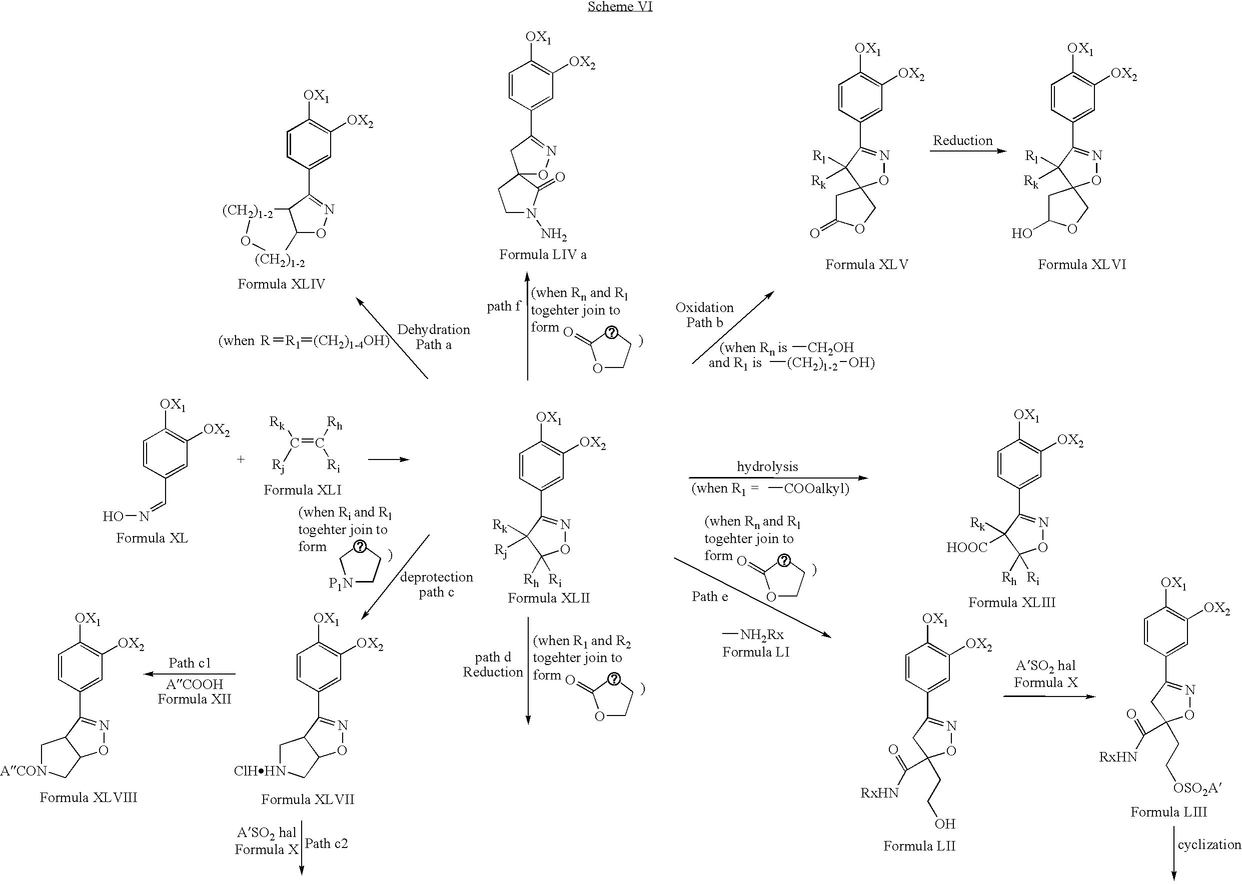

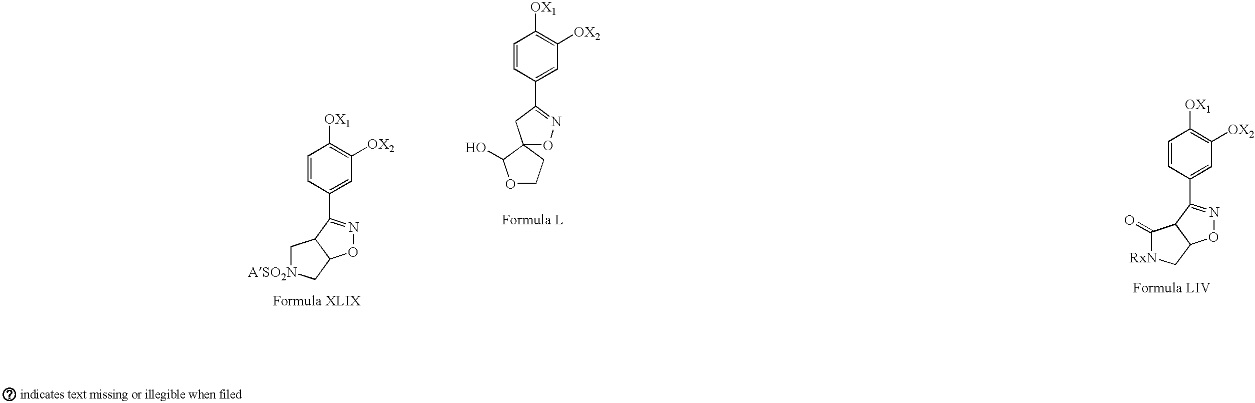

The compounds of Formulae XLIII, XLIV, XLV, XLVI, XLVII, XLVIII, XLIX, L, LI and LIV can be prepared, for example, by following the procedure as described, for example, in Scheme VI. Thus a compound of Formula XL (wherein X₁ and X₂ are the same as defined earlier) can be reacted with a compound of Formula XLI, wherein

a. Rₕ and Rᵢ may together join to form a cycloalkyl or heterocyclyl ring optionally substituted with alkaryl or oxo; Rⱼ is hydrogen or —COOalkyl and Rₖ is hydrogen,
b. Rₕ is hydrogen or —CH₂OH; Rᵢ is —(CH₂)₁₋₂OH; Rⱼ is hydrogen or —(CH₂)₁₋₂OH and Rₖ is hydrogen,
c. Rᵢ and Rⱼ together joins to form cycloalkyl or heterocyclyl ring; Rₕ and Rₖ are hydrogen;
to give a compound of Formula XLII, which can undergo hydrolysis (when Rⱼ is —COOalkyl) to give a compound of Formula XLIII,
path a: the compound of Formula XLII undergoes dehydration (when Rᵢ=Rⱼ=—(CH₂)₁₋₂OH) to give a compound of Formula XLIV;
Path b: the compound of Formula XLII undergoes oxidation (when Rₕ is —CH₂OH and Rᵢ is —(CH₂)₁₋₂OH) to give a compound of Formula XLV, which undergoes reduction to give a compound of Formula XLVI;
Path c: the compound of Formula XLII undergoes deprotection (Rᵢ and Rⱼ together joins to form

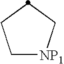

wherein represents a point of attachment and P₁ represents —C(═O)OC(CH₃)₃, —C(═O)OC(CH₃)₂CHBr₂ or —C(═O)OC(CH₃)₂CCl₃) to give a compound of Formula XLVII,
[Path c1: which can be reacted with a compound of Formula XII (wherein A″ is the same as defined earlier) to give a compound of Formula XLVIII]; or
[Path c2: which can be reacted with a compound of Formula X (wherein A′ is the same as defined earlier) to give a compound of Formula XLIX];
Path d: the compound of Formula XLII undergoes reduction (when Rₕ and Rᵢ together joins to form

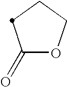

wherein represents a point of attachment) to give a compound of Formula L;
Path e: the compound of Formula XLII can be reacted with a compound of Formula LI (wherein Rₓ is the same as defined earlier) to give a compound of Formula LII, which can be reacted with a compound of Formula X to give a compound of formula LIII, which undergoes cyclisation to give a compound of Formula LIV; or
Path f: the compound of Formula XLII can be reacted with hydrazine hydrochloride to give a compound of Formula LIVa.

The reaction of a compound of Formula XL with a compound of Formula XLI to give a compound of Formula XLII can be carried out in an organic solvent, such as, for example, dichloromethane, chloroform, carbon tetrachloride, dichloromethane or tetrahydrofuran, with oxidants such as, for example, sodium hypochlorite, N-chlorosuccinimide or tert-butoxychloride, in the presence of an optional base, such as, for example, pyridine, butyl lithium, N-methylmorpholine, diisopropylethylamine or triethylamine.

The compound of Formula XLII can undergo hydrolysis (when Rⱼ is —COOalkyl) to give a compound of Formula XLIII in the presence of a basic hydrolyzing agent, such as, for example, sodium hydroxide, lithium hydroxide, potassium hydroxide, and a mixture thereof.

The compound of Formula XLII can undergo dehydration (when Rᵢ=Rⱼ=—(CH₂)₁₋₂OH) at temperature ranging from about 100-150° C. to give a compound of Formula XLIV with dehydrating agents, such as, for example, acetic anhydride, glacial acetic acid, calcium oxide or sulphuric acid.

The compound of Formula XLII can undergo oxidation (path b, when Rₕ is —CH₂OH and Rᵢ is —(CH₂)₁₋₂OH) to give a compound of Formula XLV in an organic solvent, such as, for example, dichloromethane, dichloroethane, chloroform or carbon tetrachloride, in the presence of a base for example, pyridine, triethylamine, N-methylmorpholine or diisopropylethylamine with oxidizing agents, such as, for example, chromic anhydride, sodium dichromate, potassium permanganate or potassium dichromate, pyridium chlorochromate or pyridinium dichromate

The compound of Formula XLV can undergo reduction to give a compound of Formula LXVI in an organic solvent, such as, for example, toluene, benzene or xylene, with reducing agent diisobutylaluminium hydride, sodiumborohydride, lithium aluminium hydride or sodium (bisethoxymethoxy) aluminium hydride

The compound of Formula XLII can undergo deprotection (path c, when Rᵢ and Rⱼ together joins to form

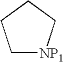

where P₁ is —C(═O)OC(CH₃)₃) to give a compound of Formula XLVII, which can be carried out in an organic solvent, such as, for example, methanol, ethanol, propanol or isopropylalcohol, in the presence of an alcoholic acid solution, such as, for example, methanolic hydrochloric acid or ethanolic hydrochloric acid.

The compound of Formula XLII can undergo deprotection (when Rᵢ and Rⱼ together joins to form

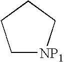

where P₁ is —C(═O)OC(CH₃)₂CHBr₂) to give a compound of Formula XLVII, which can be carried out in an organic solvent, such as, for example, ethanol, methanol, propanol or isopropylalcohol, or by hydrobromide in acetic acid.

The compound of Formula XLII can undergo deprotection (when Rᵢ and Rⱼ together joins to form

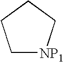

where P₁ is —C(═O)OC(CH₃)₂CCl₃) to give a compound of Formula XLVII, which can be carried out by a supernucleophile, such as, for example, lithium cobalt (I) phthalocyanine, zinc and acetic acid or cobalt phthalocyanine.

The reaction of a compound of Formula XLVII with a compound of Formula XII (path c1) to give a compound of Formula XLVIII can be carried out in an organic solvent, such as, for example, dimethylformamide, tetrahydrofuran, diethylether or dioxane in the presence of a base such as, for example, N-methylmorpholine, triethylamine, diisopropylethylamine or pyridine.

The reaction of a compound of Formula XLVII with a compound of Formula X (path c2) to give a compound of Formula XLIX can be carried out in an organic solvent, such as, for example, dichloroethane, dichloromethane, chloroform or carbon tetrachloride in the presence of a base such as, for example, triethylamine, diisopropylethylamine, N-methylmorpholine or pyridine.

The compound of formula XLII (path d, when Rₕ and Rᵢ together joins to form

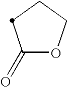

) can undergo reduction to give a compound of Formula L, in an organic solvent for example, toluene, benzene or xylene with reducing agent, such as, for example, diisobutylaluminium hydride, sodiumborohydride or lithium aluminium hydride.

The reaction of a compound of formula XLII (path e, when Rₕ and Rᵢ together joins to form

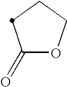

) with a compound of Formula LI to give a compound of Formula LII can be carried out in an organic solvent for example methanol, ethanol, propanol or isopropylalcohol.

The reaction of a compound of Formula LII with a compound of Formula X to give a compound of Formula LIII can be carried out in an organic solvent, such as, for example, dichloroethane, dichloromethane, chloroform or carbon tetrachloride in the presence of a base, such as, for example, triethylamine, diisopropylethylamine, N-methylmorpholine or pyridine.

The compound of Formula LIII can undergo cyclisation to give a compound of Formula LIV in an organic solvent, such as, for example, dimethylformamide, tetrahydrofuran, diethylether or dioxane, in the presence of a base, such as, for example, potassium carbonate, sodium carbonate or lithium carbonate.

The reaction of a compound of Formula XLII (path f) can be reacted with hydrazine hydrochloride to give a compound of Formula LIVa in an organic solvent, such as, for example, ethanol, methanol, propanol or isopropylalcohol.

Some representative compounds which can be prepared following Scheme VI include:
3-[3-(cyclopentyloxy)-4-methoxyphenyl]-1-oxa-2-azaspiro[4.4]non-2-ene (Compound No. 11),
Ethyl 8-benzyl-3-[3-(cyclopentyloxy)-4-methoxyphenyl]-1-oxa-2,8-diazaspiro[4.5]dec-2-ene-4-carboxylate (Compound No. 36),
3-[3-(Cyclopentyloxy)-4-methoxyphenyl]-1-oxa-2-azaspiro[4.5]dec-2-ene-4-carboxylic acid (Compound no. 37),
Ethyl 3-[3-(cyclopentyloxy)-4-methoxyphenyl]-1-oxa-2-azaspiro[4.5]dec-2-ene-4-carboxylate (Compound No. 39),
3-[3-(Cyclopentyloxy)-4-methoxyphenyl]-3a,6a-dimethyl-3aH-cyclopenta[d]isoxazole-4,6(5H,6aH)-dione (Compound No. 43),
3-[3-(Cyclopentyloxy)-4-methoxyphenyl]-6,6a-dihydrofuro[3,4-d]isoxazol-4(3aH)-one (Compound No. 45),
3-[3-(Cyclopentyloxy)-4-methoxyphenyl]-1,8-dioxa-2-azaspiro[4.5]dec-2-ene (Compound No. 52),
3-[3-(Cyclopentyloxy)-4-methoxyphenyl]-3aH-cyclopenta[d]isoxazole-4,6(5H,6aH)-dione (Compound No. 53),
3-[3-(Cyclopentyloxy)-4-methoxyphenyl]-3a,4,5,6,7,7a-hexahydro-1,2-benzisoxazole (Compound No. 56),
3-[3-(Cyclopentyloxy)-4-methoxyphenyl]-4,5,6,6a-tetrahydro-3aH-cyclopenta[d]isoxazole (Compound No. 57),
Tert-butyl 3-[3-(cyclopentyloxy)-4-methoxyphenyl]-3a,4,6,6a-tetrahydro-5H-pyrrolo[3,4-d]isoxazole-5-carboxylate (Compound No. 142),
3-[3-(Cyclopentyloxy)-4-methoxyphenyl]-3a,5,6,7a-tetrahydro-1,2-benzisoxazol-7(4H)-one (Compound No. 150).
   Some representative compounds which can be prepared following Scheme VI, path a include:
3-[3-(Cyclopentyloxy)-4-methoxyphenyl]-3a,4,6,6a-tetrahydrofuro[3,4-d]isoxazole (Compound No. 44).
   Some representative compounds which can be prepared following Scheme VI, path b include:
3-[3-(cyclopentyloxy)-4-methoxyphenyl]-1,7-dioxa-2-azaspiro[4.4]non-2-en-8-one (Compound no. 15),
3-[3-(cyclopentyloxy)-4-methoxyphenyl]-1,7-dioxa-2-azaspiro[4.4]non-2-en-8-ol (Compound No. 16).
   Some representative compounds which can be prepared following scheme VI, path c include:
3-[3-(Cyclopentyloxy)-4-methoxyphenyl]-4,5,6,6a-tetrahydro-3aH-pyrrolo[3,4-d]isoxazole (Compound No. 140)
   Some representative compounds prepared following scheme VI, path c1 include:
5-Acetyl-3-[3-(cyclopentyloxy)-4-methoxyphenyl]-4,5,6,6a-tetrahydro-3aH-pyrrolo[3,4-d]isoxazole (Compound No. 147).
   Some representative compounds which can be prepared following scheme VI, path c2 include:
3-[3-(Cyclopentyloxy)-4-methoxyphenyl]-5-(methylsulfonyl)-4,5,6,6a-tetrahydro-3aH-pyrrolo[3,4-d]isoxazole (Compound No. 148).
   Some representative compounds which can be prepared following scheme VI, path d include:
3-[3-(cyclopentyloxy)-4-methoxyphenyl]-1,7-dioxa-2-azaspiro[4.4]non-2-en-6-ol (Compound No. 1).
   Some representative compounds which can be prepared following scheme VI, path e include:
3-[3-(Cyclopentyloxy)-4-methoxyphenyl]-1-oxa-2,7-diazaspiro[4.4]non-2-en-6-one (Compound No. 42).
   Some representative compounds which can be prepared following scheme VI, path f include:
7-Amino-3-[3-(cyclopentyloxy)-4-methoxyphenyl]-1-oxa-2,7-diazaspiro[4.4]non-2-en-6-one (Compound No. 35).

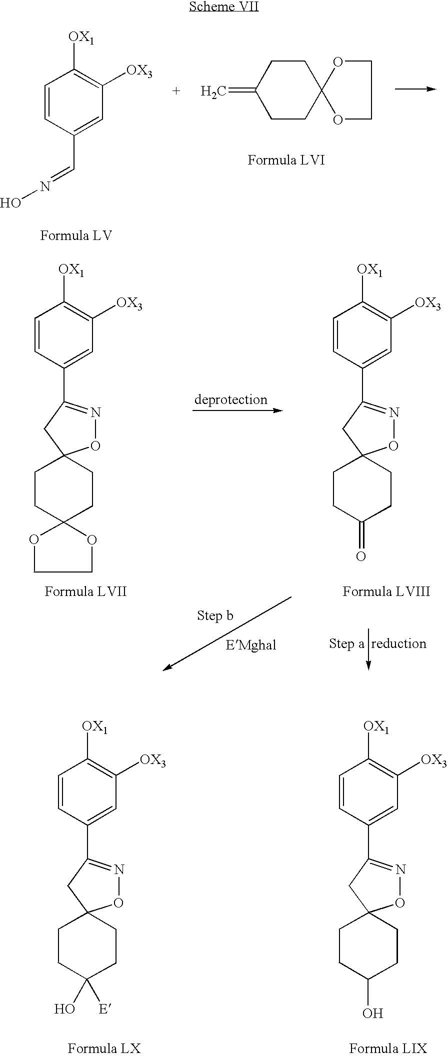

The compounds of Formulae LVIII, LIX and LX can be prepared, for example, by following the procedure as depicted in scheme VII. Thus a compound of Formula LV (wherein X₁ is the same as defined earlier and X₃ is hydrogen, alkyl, cycloalkyl, alkaryl, alkenyl, cycloalkylalkyl, heteroaryl, heterocyclyl, heteroarylalkyl, heterocyclylalkyl) can be reacted with a compound of Formula LVI to give a compound of Formula LVII, which can undergo deprotection to give a compound of Formula LVIII, which
Path a: undergoes reduction to give a compound of Formula LIX; or
Path b: can be reacted with a compound of Formula E′Mghal (wherein E′ is alkyl, alkenyl or alkynyl and hal is the same as defined earlier) to give a compound of Formula LX.

The reaction of a compound of Formula LV with a compound of Formula LVI to give a compound of Formula LVII can be carried out in an organic solvent, such as, for example, dichloromethane, chloroform, carbon tetrachloride or dichloromethane, with oxidants such as, for example, sodium hypochlorite, N-chlorosuccinimide or tert-butoxychloride, in the presence of an optional base, such as, for example, pyridine, butyl lithium, N-methylmorpholine, diisopropylethylamine or triethylamine.

The deprotection of a compound of Formula LVII to give a compound of Formula LVIII can be carried out in an organic solvent for such as, for example, dichloromethane, dichloroethane, carbon tetrachloride or chloroform, with deprotecting agent, such as, for example, trifluoroacetic acid, hydrochloric acid or sulphuric acid.

Alternatively the deprotection of a compound of Formula LVII to give a compound of Formula LVIII can also be carried out with benzyltriphenylphosphonium peroxymonosulphate or benzyltriphenylphosphonium in the presence of aluminium trichloride.

The reduction of a compound of Formula LVIII (path a) to give a compound of Formula LIX can be carried out in an organic solvent, such as, for example, methanol, ethanol or isopropylalcohol with reducing agents, such as, for example, sodium borohydride, lithium aluminium hydride or diisobutylaluminium hydride.

The reaction of a compound of Formula LVIII with a compound of Formula E′Mghal (path b) to give a compound of Formula LX can be carried out in an organic solvent, such as, for example, tetrahydrofuran, dimethylformamide, diethyl ether or dioxane.

Some representative compounds which can be prepared following Scheme VII include:
3-[3-(cyclopentyloxy)-4-methoxyphenyl]-1-oxa-2-azaspiro[4.5]dec-2-en-8-one (Compound No. 26),
   Some representative compounds which can be prepared following Scheme VII, path a include:
3-[3-(cyclopentyloxy)-4-methoxyphenyl]-1-oxa-2-azaspiro[4.5]dec-2-en-8-ol (Compound No. 24),
   Some representative compounds which can be prepared following Scheme VII, path b include:
3-[3-(Cyclopentyloxy)-4-methoxyphenyl]-8-vinyl-1-oxa-2-azaspiro[4.5]dec-2-en-8-ol (Compound No. 55),
3-[3-(Cyclopentyloxy)-4-methoxyphenyl]-8-methyl-1-oxa-2-azaspiro[4.5]dec-2-en-8-ol (Compound No. 59).

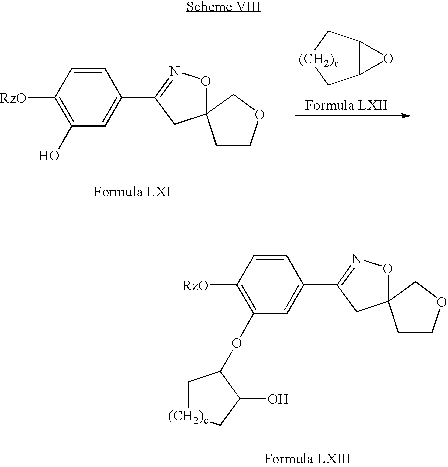

The compounds of Formulae LXIII can be prepared, for example, by the procedure as depicted, for example, in Scheme VIII. Thus, a compound of Formula LXI (wherein Rz is the same as defined earlier) can be reacted with a compound of Formula LXII (wherein c is an integer from 1-3) to give a compound of Formula LXIII.

The reaction of a compound of Formula LXI with a compound of Formula LXII to give a compound of Formula LXIII can be carried out in an organic solvent, such as, for example, dimethylformamide, tetrahydrofuran, diethylether or dioxane in the presence of a base, such as, for example, potassium carbonate, sodium carbonate or lithium carbonate.

Some representative compounds which may be prepared following Scheme VIII include:
2-[5-(1,7-Dioxa-2-azaspiro[4.4]non-2-en-3-yl)-2-methoxyphenoxy]cyclopentanol (Compound No. 137).

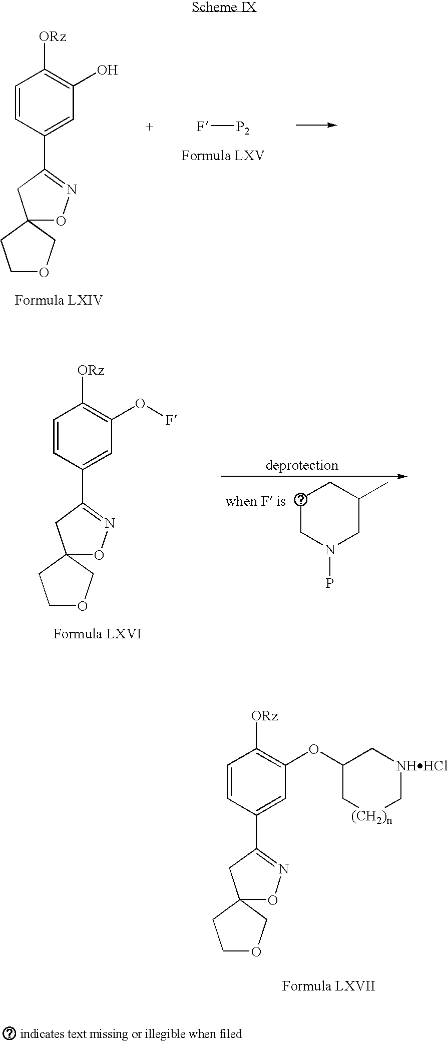

Compounds of Formulae LXVI and LXVII can be prepared, for example, by following a procedure as depicted, for example, in Scheme IX. Thus, a compound of Formula LXIV (wherein Rz is the same as defined earlier) can be reacted with a compound of Formula LXV [wherein P₂ is —O-tosyl, —O-mesyl, —O-4-bromophenylsulphonate, —O-4-nitrophenylsulfonate or —O-triflate and F′ is

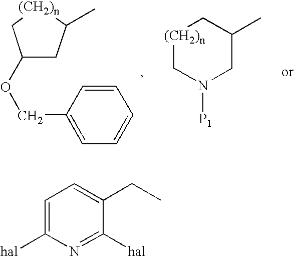

(where hal and n are the same as defined earlier and P₁ is —C(═O)OC(CH₃)₃, —C(═O)OC(CH₃)₂CHBr₂ or —C(═O)OC(CH₃)₂CCl₃)] to give a compound of Formula LXVI, which can undergo deprotection (when F′ is

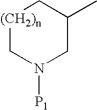

) to give a compound of Formula LXVII.

The reaction of a compound of Formula LXIV with a compound of Formula LXV to give a compound of Formula LXVI can be carried out in an organic solvent, such as, for example, dimethylformamide, tetrahydrofuran, diethyl ether or dioxane, in the presence of a base, such as, for example, potassium carbonate, sodium carbonate or lithium carbonate.

The deprotection of a compound of Formula LXVI (wherein P₁ can be —C(═O)OC(CH₃)₃) to give a compound of Formula LXVII can be carried out in an organic solvent, such as, for example, methanol, ethanol, propanol or isopropylalcohol, in the presence of an alcoholic acid solution, such as, for example, ethanolic hydrochloric acid or methanolic hydrochloric acid.

The deprotection of a compound of Formula LXVI (wherein P₁ can be —C(═O)OC(CH₃)₂CHBr₂) to give a compound of Formula LXVII can be carried out in an organic solvent, such as, for example, ethanol, methanol, propanol or isopropylalcohol or by hydrobromide in acetic acid.

The deprotection of a compound of Formula LXVI (wherein P₁ can be —C(═O)OC(CH₃)₂CCl₃) to give a compound of Formula LXVII can be carried out by a supernucleophile, such as, for example, lithium cobalt (I) phthalocyanine, zinc and acetic acid or cobalt phthalocyanine.

Some representative compounds which can be prepared following Scheme IX include:
3-(3-{[3-(Benzyloxy)cyclopentyl]oxy}-4-methoxyphenyl)-1,7-dioxa-2-azaspiro[4.4]non-2-ene (Compound No. 154),
Hydrochloride salt of 3-[4-methoxy-3-(piperidin-3-yloxy)phenyl]-1,7-dioxa-2-azaspiro[4.4]non-2-ene (Compound No. 163),
3-{3-[(2,6-Dichloropyridin-4-yl)methoxy]-4-methoxyphenyl}-1,7-dioxa-2-azaspiro[4.4]non-2-ene (Compound No. 167).

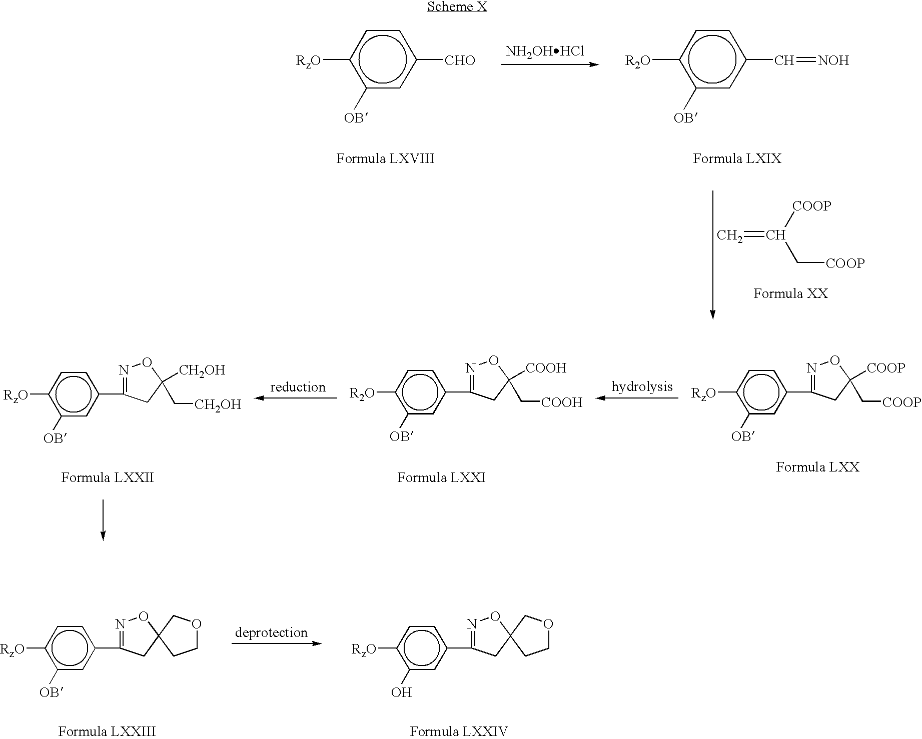

Compounds of Formulae LXXIII and LXXIV can be prepared, for example, by following the reaction sequence of Scheme X. Thus, the compound of Formula LXVIII (wherein B′ can be alkaryl) and Rz is the same as defined earlier) can be reacted with hydroxylamine hydrochloride to give a compound of Formula LXIX, which can be reacted with a compound of Formula XX to give a compound of Formula LXX, which can undergo hydrolysis to give a compound of Formula LXXI, which can undergo reduction to give a compound of Formula LXXII, which can undergo ring cyclisation to give a compound of Formula LXXIII, which can undergo deprotection to give a compound of Formula LXXIV.

The reaction of a compound of Formula LXVIII with hydroxylamine hydrochloride to give a compound of Formula LXIX can be carried out in an organic solvent, such as, for example, ethanol, methanol, propanol or isopropyl alcohol.

The compound of Formula LXIX can be reacted with a compound of Formula XX to give a compound of Formula LXX in an organic solvent, such as, for example, dichloromethane, chloroform, carbon tetrachloride or dichloromethane with oxidants such as, for example, sodium hypochlorite, N-chlorosuccinimide or tert-butoxychloride, in the presence of an optional base, such as, for example, pyridine, butyl lithium, N-methylmorpholine, diisopropylethylamine or triethylamine

The hydrolysis of a compound of Formula LXX to give a compound of Formula LXXI can be carried out in a solvent system, such as, for example, tetrahydrofuran, methanol, dioxane or ethanol, in water in the presence of base, such as, for example, lithium hydroxide, sodium hydroxide or potassium hydroxide.

The compound of Formula LXXI can undergo reduction to give a compound of Formula LXXII in an organic solvent, such as, for example, tetrahydrofuran, dimethylformamide, dioxane or diethyl ether, with reducing agent, such as, for example, sodium borohydride or sodium cyanoborohydride.

The compound of Formula LXXII can undergo ring cyclisation to give a compound of Formula LXXIII in an organic solvent, such as, for example in an organic solvent for example, tetrahydrofuran, dimethylformamide, dioxane or diethyl ether in the presence of a redox couple. The oxidizing part of the redox couple can be selected from, for example, diisopropylazodicarboxylate (DIAD), diethylazodicarboxylate (DEAD), N,N,N′,N′-tetramethylazodicarboxylate (TMAD), 1,1′-(azodicarbonyl) dipiperidine (ADDP), cyanomethylenetributylphosphorane (CMBP), 4,7-dimethyl-3,5,7-hexahydro-1,2,4,7-tetrazocin-3,8-dione (DHTD) or N,N,N′,N,′-tetraisopropylazodicarboxamide (TIPA). The reduction part of the redox couple can be phosphine, for example, trialkylphosphine (such as tributylphosphine), triarylphosphine (such as triphenylphosphine), tricycloalkylphosphine (such as triscyclohexylphosphine) or tetraheteroarylphosphine. The phosphine reagents with a combination of aryl, alkyl or heteroaryl substituents may also be used (such as diphenylpyridylphosphine).

The compound of Formula LXXIII can be deprotected to give a compound of Formula LXXIV in an organic solvent, such as, for example, methanol, ethanol, propanol or isopropylalcohol, with a deprotecting agent, such as, for example, palladium on carbon.

Some representative compounds which can be prepared following the procedure as described in Scheme X include:
2-(Difluoromethoxy)-5-(1,7-dioxa-2-azaspiro[4.4]non-2-en-3-yl)phenol (Compound No. 41)

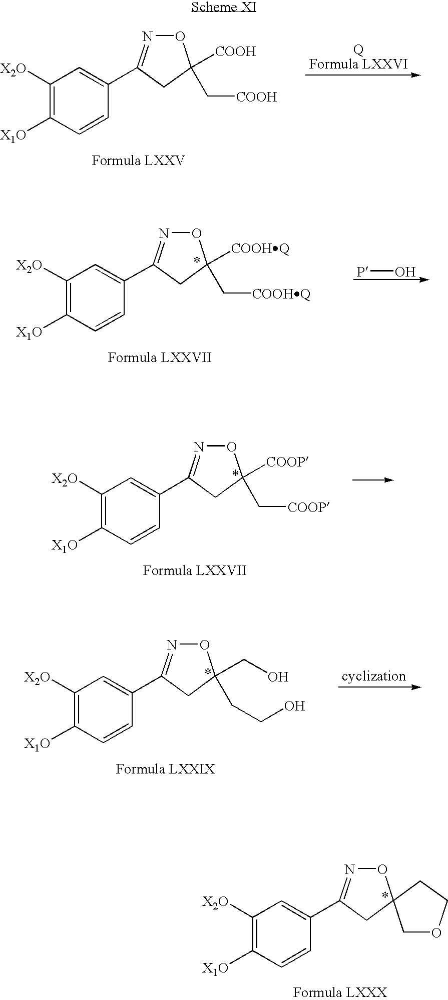

Compounds of Formula LXXX can be prepared by, for example, following a procedure as depicted in Scheme XI. Thus a compound of Formula LXXV (wherein X₁ and X₂ are the same as defined earlier) can be reacted with a compound of Formula LXXVI (wherein Q is a chiral resolving agent, for example, L-Ephederine, D-Ephederine, Brucine, (1S,2R) (−)-cis-1-amino-2-indanol, (1R2S) (+)-cis-1-amino-2-indanol, (1R,2R)-(−)-1,2-diamino cyclohexane or (1S,2S)-(+)-1,2-diamino cyclohexane or α-methylbenzylamine) to give a compound of Formula LXXVII, which can undergo protection with a compound of Formula P′-OH to give a compound of Formula LXXVIII (wherein P′ is alkyl), which can undergo reduction to give a compound of Formula LXXIX, which undergoes cyclisation to give a compound of Formula LXXX (wherein LXXX represents S-isomer when L-Ephidrine is used or R-isomer when D-Ephidrine is used).

The compound of Formula LXXV can be reacted with a compound of Formula LXXVI to give a compound of Formula LXXVII in an organic solvent such as, for example, acetone, dichloromethane or chloroform.

The protection of a compound of Formula LXXVII with a compound of Formula P′-OH to give a compound of Formula LXXVIII can be carried out with halogenating agents such as, for example, thionyl chloride, phosphorous pentachloride or phosphorous trichloride.

The compound of Formula LXXVIII undergoes reduction to give a compound of Formula LXXIX in an organic solvent, such as, for example, tetrahydrofuran, dimethylformamide, diethyl ether or dioxane, with reducing agent, such as, for example, sodiumboro hydride, lithium aluminium hydride or lithiumboro hydride.

Alternatively, the compound of Formula LXXIX can also be prepared by reducing free acid form of compound of Formula LXXVII.

The compound of Formula LXXIX can undergo cyclisation to give a compound of Formula LXXX in an organic solvent, such as, for example in an organic solvent for example, tetrahydrofuran, dimethylformamide, dioxane or diethyl ether, in the presence of a redox couple. The oxidizing part of the redox couple can be, for example, diisopropylazodicarboxylate (DIAD), diethylazodicarboxylate (DEAD), N,N,N′,N′-tetramethylazodicarboxylate (TMAD), 1,1′-(azodicarbonyl) dipiperidine (ADDP), cyanomethylenetributylphosphorane (CMBP), 4,7-dimethyl-3,5,7-hexahydro-1,2,4,7-tetrazocin-3,8-dione (DHTD) or N,N,N′,N,′-tetraisopropylazodicarboxamide (TIPA). The reduction part of the redox couple can be phosphine, for example, trialkylphosphine (such as tributylphosphine), triarylphosphine (such as triphenylphosphine), tricycloalkylphosphine (such as triscyclohexylphosphine) or tetraheteroarylphosphine. The phosphine reagents with a combination of aryl, alkyl or heteroaryl substituents may also be used (such as diphenylpyridylphosphine).

Some representative compounds which may be prepared following Scheme XI include:
(R)-3-[3-(cyclopentyloxy)-4-methoxyphenyl]-1,7-dioxa-2-azaspiro[4.4]non-2-ene (Compound No. 30),
(S)-3-[3-(cyclopentyloxy)-4-methoxyphenyl]-1,7-dioxa-2-azaspiro[4.4]non-2-ene (Compound No. 124).

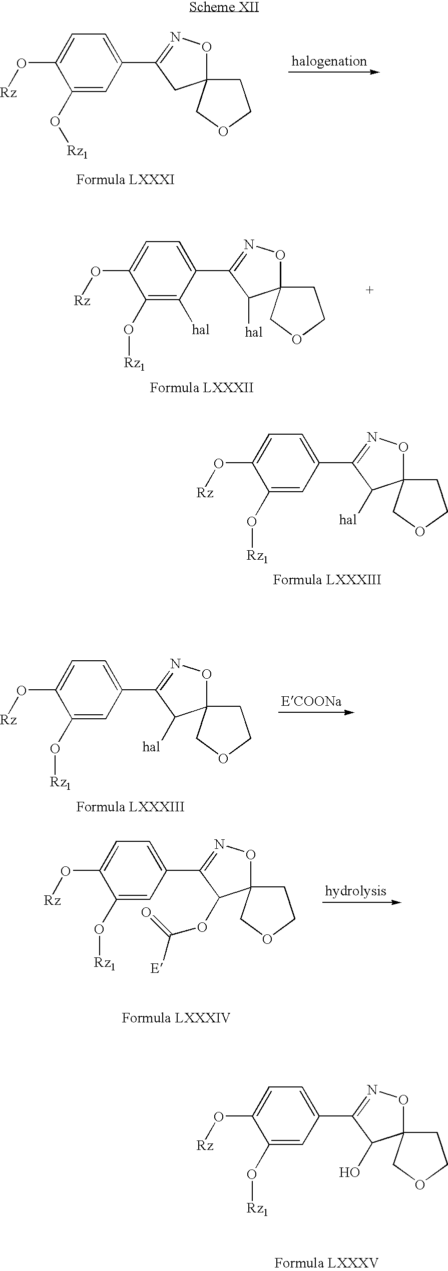

The compounds of Formulae LXXXIV and LXXXV can be prepared by, for example, following a procedure as depicted, for example, in Scheme XII. Thus a compound of Formula LXXXI (wherein Rz and Rz1 are the same as defined earlier) can undergo halogenation to give compounds of Formula LXXXII and LXXXIII. The compound of Formula LXXXIII can be reacted with a compound of Formula E′COONa (wherein E′ is the same as defined earlier) to give a compound of Formula LXXXIV, which can be hydrolysed to give a compound of Formula XXXV.

The halogenation of a compound of Formula LXXXI to give a compound of Formula LXXXII and LXXXIII can be carried out in an organic solvent, such as, for example, chloroform, carbon tetrachloride, dichloromethane or dichloroethane, in the presence of radical initiator, such as, for example, azoisobutyronitrile (AIBN) or di-tert-butyl peroxide (BOOB), with halogenating agent, such as, for example, N-bromosuccinimide, N-chlorosuccinimide or N-iodosuccinimide.

The reaction of a compound of Formula LXXXIII with a compound of Formula E′COONa to give a compound of Formula LXXXIV can be carried out in an organic solvent, such as, for example, dimethylformamide, tetrahydrofuran, diethyl ether or dioxane.

The hydrolysis of a compound of Formula LXXXIV to give a compound of Formula LXXXV can be carried out in an organic solvent, such as, for example, methanol, ethanol or isopropylalcohol, in the presence of a base, such as, for example, potassium carbonate, sodium carbonate or lithium carbonate.

Some representative compounds which may be prepared following Scheme XII include:
3-[3-(cyclopentyloxy)-4-methoxyphenyl]-1,7-dioxa-2-azaspiro[4.4]non-2-en-4-ol (Compound No. 29),
4-Bromo-3-[3-(cyclopentyloxy)-4-methoxyphenyl]-1,7-dioxa-2-azaspiro[4.4]non-2-ene (Compound No. 149)

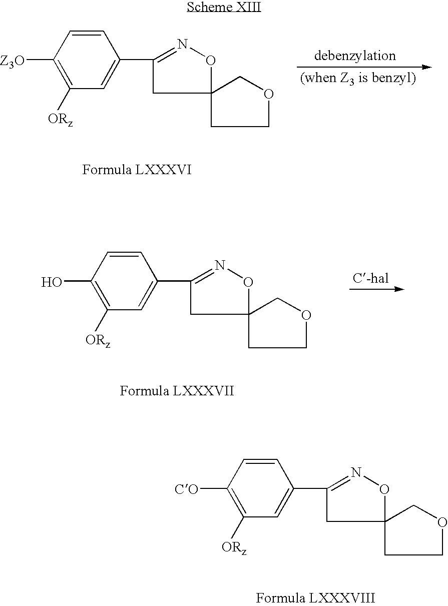

The compound of Formula LXXXVIII can be prepared, for example, by reaction sequence as depicted, for example, in Scheme XIII. Thus, a compound of Formula LXXXVI can be debenzylated (wherein Z₃ can be alkaryl) to give a compound of Formula LXXXVII, which can be reacted with a compound of Formula C′-hal to give a compound of Formula LXXXVIII.

The debenzylation of a compound of Formula LXXXVI to give a compound of formula LXXXVII can be carried out in an organic solvent, such as, for example, methanol, ethanol, propanol or isopropylalcohol, with a deprotecting agent, such as, for example, using hydrogen and palladium on carbon, or under catalytic hydrogenation transfer conditions of ammonium formate and palladium on carbon.

The reaction of a compound of Formula LXXXVII with a compound of Formula C′-hal to hive a compound of Formula LXXXVIII can be carried out in an organic solvent, such as, for example, dimethylformamide, tetrahydrofuran, diethyl ether or dioxane, in the presence of a base such as, for example, potassium carbonate, sodium carbonate or lithium carbonate.

Some representative compounds which can be prepared following Scheme XIII include:
3-[3-methoxy-4-(2-morpholin-4-ylethoxy)phenyl]-1,7-dioxa-2-azaspiro[4.4]non-2-ene (Compound No. 14),

### EXAMPLES

### Synthesis of Ethyl Cyclohexylideneacetate

To slurry of triethyl phosphonoacetate (5.05, 22.3 mmole) in tetrahydrofuran (5 ml) at 20° C. was added sodium hydride (0.892 g, 22.3 mmole) portionwise with constant stirring followed by the addition of cyclohexanone (1.87 ml, 22.3 mmole) in tetrahydrofuran (2 ml) dropwise. The reaction mixture was stirred for 1 hour. The mixture was diluted with water and extracted with ethyl acetate, dried over anhydrous sodium sulphate and concentrated under reduced pressure. The residue thus obtained was purified by column chromatography to furnish the title compound. Yield: 2.5 gm

### Synthesis of tert-Butyl 2,5-dihydro-1H-pyrrole-1-carboxylate

To a solution of the compound 2,5-dihydro-1H-pyrrole (commercially available) (400 mg, 0.0078 mol) in dichloromethane (50 ml) was added triethyl amine (1.75 g, 0.0173 mol) and cooled the mixture to 0° C. followed by the addition of di-tert-butoxy carbonyl anhydride (1.89 g, 0.00868 mol) dropwise. The reaction mixture was stirred for overnight. The mixture was extracted with dichloromethane. The organic layer was washed with brine, dried over anhydrous sodium sulphate and concentrated under reduced pressure to furnish the title compound. Yield: 1 g.

### Synthesis of 4-(Difluoromethoxy)3-benzyloxybenzaldehyde

To a solution of 3-hydroxy-4-difluoromethoxymethoxy-benzaldehyde (1 eq) was taken in dimethylformamide (10 mL), was added potassium iodide (0.1 eq) and potassium carbonate (2 eq). The reaction mixture was stirred at 70° C. and cyclopentyl bromide (2 eq) was added dropwise. The resulting reaction mixture was stirred at 70-80° C. for 16 hours. The reaction mixture was cooled and diluted with water, extracted with ethyl acetate and washed with saturated solution of sodium chloride. The organic solvent was removed under reduced pressure. The residue thus obtained was purified by column chromatography to furnish the title compound.

### Synthesis of 3-(Benzyloxy)cyclopentanol

To a stirred solution of cyclopentane-1,3-diol (1.0 g, 9.80 mmol) and silver oxide (3.41 g, 14.7 mmol) in dichloromethane (300 ml) was added benzyl bromide (1.05 ml, 8.82 mmol) under dark conditions at room temperature and stirred the reaction mixture for 44 hours. The reaction mixture was filtered through celite pad and washed with dichloromethane. The combined organic layer was washed with water, dried over anhydrous sodium sulphate and concentrated under reduced pressure. The residue thus obtained was purified by column chromatography to furnish the title compound. Yield: 0.38 g.

### Synthesis of tert-Butyl 3-hydroxypiperidine-1-carboxylate

To a mixture of 3-hydroxy piperidine (4.0 gm, 39.6 mmole) and triethyl amine (11.0 ml, 79.0 mmole) in dichloromethane (70 ml) at 0° C. was added tert-butoxy carbonyl anhydride (10.4 gm, 47.4 mmole) and stirred the reaction mixture at room temperature for 12 hrs. The reaction mixture was washed with water and saturated sodium chloride solution, dried over anhydrous sodium sulphate and concentrated under reduced pressure to furnish the title compound. Mass (m/z): 128 (MH⁺−tert. butanol).

### Synthesis of 2,6-dichloropyridin-3-yl)methanol

To a solution of the compound 2,6-dichloronicotinic acid (0.5 g, 2.6 mmol) in tetrahydrofuran (10 ml) at 0° C. was added sodium borohydride (0.29 g, 7.8 mmol) portion wise and stirred the reaction mixture at room temperature for 30 minutes. The resulting reaction mixture was again cooled to 0° C. followed by the addition of etheral solution of boron trifluoride (1.1 ml, 7.8 mmole) dropwise and stirred the mixture at room temperature for overnight. The reaction mixture was quenched with aqueous sodium hydroxide (1N) and the solvent was evaporated under reduced pressure to furnish the title compound. The residue thus obtained was diluted with water and extracted with ethyl acetate. The organic layer was washed with water and brine, dried over anhydrous sodium sulphate and concentrated under reduced pressure to furnish the title compound. Yield: 0.44 g.

### Synthesis of 2,6-dichloropyridin-3-yl)methyl toluenesulphonate

To a stirred solution of the compound 2,6-dichloropyridin-3-yl)methanol (0.4 g, 2.25 mmol), 4-dimethylaminopyridine (0.028 g, 0.225 mmol) and triethylamine (0.62 ml, 4.5 mmol) in dichloromethane (20 ml) was added p-toluene sulphonyl chloride (0.64 g, 3.75 mmol) portion wise at 0-5° C. and stirred the reaction mixture at room temperature for overnight. The mixture was diluted with dichloromethane, washed with water and brine, dried over anhydrous sodium sulphate and concentrated under reduced pressure to furnish the title compound. Yield: 0.725 g.

The following compounds can be prepared analogously,

3-(Benzyloxy)cyclopentyl methanesulfonate: Mass (m/z): 347.0 (M⁺+1).

Tert-butyl 3-[(methylsulfonyl)oxy]piperidine-1-carboxylate: Mass (m/z): 280.0 (M⁺+1).

### Example 1

### Tert-butyl 3-[3-(cyclopentyloxy)-4-methoxyphenyl]-1-oxa-2,7-diazaspiro[4.5]dec-2-ene-7-carboxylate

### Compound No. 21

### Step a: Synthesis of 3-oxo-piperidine-1-carboxylic acid tert-butyl ester

To a solution of the compound 3-hydroxy-piperidinyl-1-carboxylic acid tert-butyl ester (7.5 gm, 37.3 mmole) in dichloromethane (100 mL) was added celite (5.0 gm) and stirred at room temperature for 10 minutes. Pyridinium chlorochromate (9.57 gm, 44.4 mmole) was added portionwise over a period of 5 minutes. The reaction mixture was stirred at room temperature for 3 hours. Dichloromethane was removed under reduced pressure followed by the addition of ethyl acetate. The resulting reaction mixture was again stirred for 10 minutes and filtered through celite pad. The organic layer was removed under reduced pressure. The residue thus obtained was purified by column chromatography to furnish the title compound. Yield: 1.4 gm, 19%

### Step b: Synthesis of 3-methylene-piperidine-1-carboxylic acid tert-butyl ester

The solution of a compound triphenylmethylphosphonium iodide (7.12 gm, 17.6 mmole), potassium tert-butoxide (1.58 gm, 14.1 mmole) in tetrahydrofuran (100 mL) was stirred at −78° C. for 20 minutes and then at room temperature for 1 hour. To the resulting reaction mixture was added a solution of the compound obtained from step a above (1.4 gm, 7.04 mmole) in tetrahydrofuran (50 mL) at 0° C. The resulting reaction mixture was stirred at room temperature for 10 min. followed by diluting it with water. Tetrahydrofuran was evaporated under reduced pressure, extracted with ethyl acetate, washed with anhydrous sodium sulphate and concentrated under reduced pressure. The residue thus obtained was purified by column chromatography to furnish the title compound. Yield: 0.6 gm.

### Step c: Synthesis of tert-butyl 3-[3-(cyclopentyloxy)-4-methoxyphenyl]-1-oxa-2,7-diazaspiro[4.5]dec-2-ene-7-carboxylate (Compound No. 21)

The compound obtained from step b above (0.4 gm, 2.04 mmole) and 3-cyclopentyloxy-4-methoxy-benzaldehyde oxime (0.53 gm, 2.25 mmole) was taken in dichloromethane (20%) in chloroform followed by the addition of pyridine (2 drops). The reaction mixture was stirred at room temperature for 10 minutes followed by the addition of sodium hypochlorite (2 mL) dropwise. The resulting reaction mixture was stirred at room temperature for 4 hours. Tetrahydrofuran was evaporated under reduced pressure followed by diluting it with water. The compound was extracted with ethyl acetate, washed with brine, dried over anhydrous sodium sulphate and evaporated under reduced pressure. The residue thus obtained was purified by column chromatography to furnish the title compound. Yield: 0.26 gm. Mass (m/z): 431 (M⁺+1).

### Example 2

### Hydrochloride salt of 3-[3-(cyclopentyloxy)-4-methoxyphenyl]-1-oxa-2,7-diazaspiro[4.5]dec-2-ene

### Compound No. 25

To a solution of Compound No. 21 (0.18 gm, 0.42 mmole) in dichloromethane (50 mL), was added methanolic hydrochloric acid (4.2 ml, 8.37 mmole) at 0° C. and the reaction mixture was stirred at room temperature for 7 hours. The resulting reaction mixture was concentrated under reduced pressure, washed with saturated sodium bicarbonate solution and extracted with ether. Organic layer was concentrated under reduced pressure. The residue thus obtained was purified by column chromatography to furnish the title compound. Yield: 0.19 g. Mass (m/z): 331 (M⁺+1).

### Example 3

### 3-[3-(cyclopentyloxy)-4-methoxyphenyl]-N-(butyl)-1-oxa-2,7-diazaspiro[4.4]non-2-ene-7-carboxamide

### Compound No. 5

To a solution of the compound hydrochloride salt of 3-[3-(cyclopentyloxy)-4-methoxyphenyl]-1-oxa-2,7-diazaspiro[4.4]non-2-ene (disclosed in our copending patent application U.S. Ser. No. 60/498,947) (100 mg, 0.2840 mmol) in dichloroethane (2 mL) was added triethylamine (0.061 ml, 0.568 mmol) at room temperature followed by the addition of 1-isocyanatobutane dropwise (42.1 mg, 0.420 mmol). The reaction mixture was stirred at room temperature for 8 hours. The resulting mixture was quenched with aqueous sodium bicarbonate solution and dichloroethane was removed under reduced pressure. The mixture was extracted with ethyl acetate. The organic extracts were separated, washed with water and brine and dried over anhydrous sodium sulphate. They were then filtered and concentrated under reduced pressure. The residue thus obtained was purified by column chromatography using 80% ethyl acetate in hexane solvent mixture as eluent to furnish the title compound. Yield: 50 mg. Mass (m/z): 416.17 (M⁺+1).

Analogues of 3-[3-(cyclopentyloxy)-4-methoxyphenyl]-N-(butyl)-1-oxa-2,7-diazaspiro[4.4]non-2-ene-7-carboxamide (Compound No. 5) described below, can be prepared analogously,
N-4-Fluoro phenyl-3-[3-(cyclopentyloxy)-4-methoxyphenyl]-1-oxa-2,7-diazaspiro[4.4]non-2-ene-7-carboxamide (Compound No. 2),

Mass (m/z): 454.25 (M⁺+1).
N-Butyl-3-[3-(cyclopentyloxy)-4-methoxyphenyl]-1-oxa-2,8-diazaspiro[4.5]dec-2-ene-8-carboxamide (Compound No. 9),

Mass (m/z): 430.25 (M⁺+1).
N-Benzyl-3-[3-(cyclopentyloxy)-4-methoxyphenyl]-1-oxa-2,7-diazaspiro[4.4]non-2-ene-7-carboxamide (Compound No. 19),

Mass (m/z): 450.25 (M⁺+1).
N-Benzyl-3-[3-(cyclopentyloxy)-4-methoxyphenyl]-1-oxa-2,8-diazaspiro[4.5]dec-2-ene-8-carboxamide (Compound No. 32),

Mass (m/z): 464.0 (M⁺+1).
3-[3-(Cyclopentyloxy)-4-methoxyphenyl]-1-oxa-2,8-diazaspiro[4.5]dec-2-ene-8-carboxamide (Compound No. 143),

Mass (m/z): 388.19 (M⁺+1).
N-Butyl-3-[3-(cyclopentyloxy)-4-methoxyphenyl]-1-oxa-2,7-diazaspiro[4.5]dec-2-ene-7-carboxamide (Compound No. 144).

### Example 4

### 3-[3-(cyclopentyloxy)-4-methoxyphenyl]-N,N-dimethyl-1-oxa-2,7-diazaspiro[4.4]non-2-ene-7-sulfonamide

### Compound No. 4

To a solution of the compound hydrochloride salt of 3-[3-(cyclopentyloxy)-4-methoxyphenyl]-1-oxa-2,7-diazaspiro[4.4]non-2-ene (disclosed in our copending patent application U.S. Ser. No. 60/498,947) (100 mg, 0.2840 mmol) in dichloromethane (1 mL) was added triethylamine (71.7 mg, 0.7102 mmol) at room temperature followed by the addition of dimethylsulfamoylchloride (61 mg, 0.054 ml, 0.426 mmol). The reaction mixture was stirred at room temperature for 10 hours. The resulting mixture was quenched with aqueous sodium bicarbonate solution and extracted with dichloromethane followed by the removal of dichloromethane under reduced pressure. The organic extracts were separated, washed with water and brine and dried over anhydrous sodium sulphate. They were then filtered and concentrated under reduced pressure to furnish the title compound. Yield: 70 mg. Mass (m/z): 424.19 (M⁺+1).

Analogues of 3-[3-(cyclopentyloxy)-4-methoxyphenyl]-N,N-dimethyl-1-oxa-2,7-diazaspiro[4.4]non-2-ene-7-sulfonamide (Compound No. 4) described below, can be prepared analogously,
3-[3-(Cyclopentyloxy)-4-methoxyphenyl]-8-(methylsulfonyl)-1-oxa-2,8-diazaspiro[4.5]dec-2-ene (Compound No. 10),

Mass (m/z): 409.08 (M⁺+1).
3-[3-(Cyclopentyloxy)-4-methoxyphenyl]-7-(methylsulfonyl)-1-oxa-2,7-diazaspiro[4.5]dec-2-ene (Compound No. 145)

Mass (m/z): 409.22 (M⁺+1).

### Example 5

### 3-[3-(cyclopentyloxy)-4-methoxyphenyl]-7-(tetrahydrofuran-3-ylcarbonyl)-1-oxa-2,7-diazaspiro[4.4]non-2-ene

### Compound No. 3

To a solution of the compound hydrochloride salt of 3-[3-(cyclopentyloxy)-4-methoxyphenyl]-1-oxa-2,7-diazaspiro[4.4]non-2-ene (disclosed in our copending patent application U.S. Ser. No. 60/498,947) (100 mg, 0.2840 mmol) in dimethylformamide (1 mL) was added tetrahydrofuran-3-carboxylic acid (36.24 mg, 0.31249 mmol). The reaction mixture was cooled to 0° C. stirred followed by the addition of N-methylmorpholine (0.187 ml, 1.704 mmol) and hydroxybenzotriazole (38.38 mg, 0.284 mmol). The resulting mixture was stirred for 30 minutes at the same temperature followed by the addition of 1-(3-dimethylaminopropyl)-3-ethylcarbodiimide hydrochloride (60 mg, 0.3124 mmol). The mixture was again stirred for 10 hours. The resulting mixture was diluted with water and extracted with ethyl acetate. The organic extracts were separated, washed with water and brine and dried over anhydrous sodium sulphate. They were then filtered and concentrated under reduced pressure and the residue thus obtained was purified by column chromatography using 5% methanol in ethyl acetate solvent mixture as eluent to furnish the title compound. Yield: 80 mg. Mass (m/z): 415.22 (M⁺+1).

Analogues of 3-[3-(cyclopentyloxy)-4-methoxyphenyl]-7-(tetrahydrofuran-3-ylcarbonyl)-1-oxa-2,7-diazaspiro[4.4]non-2-ene (Compound No. 3) described below, can be prepared analogously,
Hydrochloride salt of 3-[3-(cyclopentyloxy)-4-methoxyphenyl]-8-prolyl-1-oxa-2,8-diazaspiro[4.5]dec-2-ene (Compound No. 7)

Mass (m/z): 428.24 (M⁺+1).
3-[3-(Cyclopentyloxy)-4-methoxyphenyl]-7-(cyclopropylcarbonyl)-1-oxa-2,7-diazaspiro[4.4]non-2-ene (Compound No. 18)

Mass (m/z): 385.23 (M⁺+1).
7-Acetyl-3-[3-(cyclopentyloxy)-4-methoxyphenyl]-1-oxa-2,7-diazaspiro[4.4]non-2-ene (Compound No. 20)

Mass (m/z): 359.25 (M⁺+1).
8-Acetyl-3-[3-(cyclopentyloxy)-4-methoxyphenyl]-1-oxa-2,8-diazaspiro[4.5]dec-2-ene (Compound No. 48)

Mass (m/z): 373.22 (M⁺+1).
8-(Cyclopentylcarbonyl)-3-[3-(cyclopentyloxy)-4-methoxyphenyl]-1-oxa-2,8-diazaspiro[4.5]dec-2-ene (Compound No. 49)

Mass (m/z): 427.21 (M⁺+1).
7-(Cyclopentylcarbonyl)-3-[3-(cyclopentyloxy)-4-methoxyphenyl]-1-oxa-2,7-diazaspiro[4.5]dec-2-ene (Compound No. 141)

Mass (m/z): 427.30 (M⁺+1).
7-Acetyl-3-[3-(cyclopentyloxy)-4-methoxyphenyl]-1-oxa-2,7-diazaspiro[4.5]dec-2-ene (Compound No. 155)

Mass (m/z): 373.07 (M⁺+1).

### Example 6

### 2-{3-[3-(cyclopentyloxy)-4-methoxyphenyl]-1-oxa-2,7-diazaspiro[4.4]non-2-en-7-yl}acetamide

### Compound No. 6

To a solution of the compound hydrochloride salt of 3-[3-(cyclopentyloxy)-4-methoxyphenyl]-1-oxa-2,7-diazaspiro[4.4]non-2-ene (disclosed in our copending patent application U.S. Ser. No. 60/498,947) (99 mg, 0.2553 mmol) in dimethylformamide (2 ml), was added potassium carbonate (70 mg, 0.5106 mmol) and heated the reaction mixture to 60° C. To the resulting mixture was added bromoacetamide (42.5 mg, 0.306 mmol) dropwise and stirred the reaction mixture at 60° C. for 10 hours. The reaction mixture was diluted with water and extracted with ethyl acetate. The organic extracts were collected, washed with brine, dried over anhydrous sodium sulphate and concentrated under reduced pressure. The residue thus obtained was purified by column chromatography using 5% methanol in ethyl acetate solvent mixture as eluent to furnish the title compound. Yield: 80 mg. Mass (m/z): 374.20 (M⁺+1).

Analogues of 2-{3-[3-(cyclopentyloxy)-4-methoxyphenyl]-1-oxa-2,7-diazaspiro[4.4]non-2-en-7-yl}acetamide (Compound No. 6) described below, can be prepared analogously,
3-[3-Cyclopentyloxy)-4-methoxyphenyl]-8-(2-morpholin-4-ylethyl)-1-oxa-2,8-diazaspiro[4.5]dec-2-ene (Compound No. 8)

Mass (m/z): 444.25 (M⁺+1),
3-[3-(Cyclopentyloxy)-4-methoxyphenyl]-7-isopropyl-1-oxa-2,7-diazaspiro[4.4]non-2-ene (Compound No. 17)

Mass (m/z): 359.25 (M⁺+1),
3-[3-(Cyclopentyloxy)-4-methoxyphenyl]-8-(cyclopropylmethyl)-1-oxa-2,8-diazaspiro[4.5]dec-2-ene (Compound No. 31)

Mass (m/z): 385.16 (M⁺+1),
8-Benzyl-3-[3-(cyclopentyloxy)-4-methoxyphenyl]-1-oxa-2,8-diazaspiro[4.5]dec-2-ene (Compound No. 38)

Mass (m/z): 421.22 (M⁺+1),
3-[3-(Cyclopentyloxy)-4-methoxyphenyl]-8-(2-piperidin-1-ylethyl)-1-oxa-2,8-diazaspiro[4.5]dec-2-ene (Compound No. 50)

Mass (m/z): 442.24 (M⁺+1),
3-[3-(Cyclopentyloxy)-4-methoxyphenyl]-8-ethyl-1-oxa-2,8-diazaspiro[4.5]dec-2-ene (Compound No. 54)

Mass (m/z): 359.21 (M⁺+1).

### Example 7

### 4-(1,7-Dioxa-2-azaspiro[4.4]non-2-en-3-yl)benzene-1,2-diol

### Compound No. 34

### Step a: Synthesis of 3,4-bis(benzyloxy)benzaldehyde

To a solution of the compound 3,4-dihydroxybenzaldehyde (25 g, 181.1 mmol) in dimethylformamide (150 ml) was added benzyl chloride (114.6 g, 905.7 mmol) and potassium carbonate (124.9 g, 905.7 mmol). The reaction mixture was stirred for 20 hours at 65-70° C. which subsequently cooled and diluted with toluene (50 ml) and filtered. The solid thus obtained was washed with toluene. The organic extracts were collected and washed with sodium hydroxide, water and dried over anhydrous sodium sulphate. The organic layer was concentrated under reduced pressure and the solid thus formed was added in hexane with vigorous stirring. Filtered and dried under reduced pressure. Yield: 49.732 g.

### Step b: Synthesis of 3,4-bis(benzyloxy)benzaldehyde oxime

Hydroxylamine hydrochloride (42.8 g, 616.3 mmole) and sodium acetate (50.5 g, 616.3 mmole) was added to a stirred solution of compound obtained from step a above (49.0 g, 154.0 mmole) in ethanol (200 ml). The reaction mixture was stirred at room temperature for 50 minutes. Ethanol was evaporated under reduced pressure, which was diluted with water (100 ml) and the organic compound was extracted with ethyl acetate (3×100 ml). The ethyl acetate layer was dried over anhydrous sodium sulphate, filtered and concentrated under reduced pressure to afford the title compound.

### Step c: Synthesis of methyl 3-[3,4-bis(benzyloxy)phenyl]-5-(2-methoxy-2-oxoethyl)-4,5-dihydroisoxazole-5-carboxylate

Dimethyl 2-methylenesuccinate (38.5 g, 122.0 mmole) was added to the solution of compound obtained from step b above (40.6 h, 122.0 mmole) in tetrahydrofuran (240 mL), and the resulting reaction mixture was stirred at room temperature. Sodium hypochlorite (250 mL) was added slowly to the mixture thus obtained over the period of 20 minutes and the reaction mixture was allowed to stir at room temperature overnight. A second lot of sodium hypochlorite (100 mL) was again added to it and stirred for 2 hours at room temperature. Tetrahydrofuran was evaporated off and the organic compound was extracted with ethyl acetate twice. The organic layer was concentrated to furnish the title compound. Yield: 56.3 g.

### Step d: Synthesis of 3-[3,4-bis(benzyloxy)phenyl]-5-(carboxymethyl)-4,5-dihydroisoxazole-5-carboxylic acid

The compound obtained from step c above (0.70 gm, 2.102 mmole, 1 eq.) was dissolved in tetrahydrofuran (15 mL) and lithium hydroxide in water solution (4.8 mL of 0.5 M aqueous solution, 2.4 mmoles, 1.2 eq) was added. The mixture was stirred for 1 hour at room temperature and an additional amount of lithium hydroxide in water solution (1.9 mL, 0.5 M) was added. The mixture was stirred for 2 hour 35 minutes. Solvent was removed under reduced pressure and the residue thus obtained was diluted with water and acidified with drop of concentrated hydrochloric acid. The organic compound was extracted with ethyl acetate, washed with brine, dried over anhydrous sodium sulphate and finally concentrated under reduced pressure to afford the title organic compound with a yield of 0.500 g.

### Step e: Synthesis of 2-[3-[3,4-bis(benzyloxy)phenyl]-5-(hydroxymethyl)-4,5-dihydroisoxazol-5-yl]ethanol

To a solution of sodium borohydride (3 eq) in tetrahydrofuran, was added a solution of the compound obtained from step d above (1 eq) in tetrahydrofuran. To the resulting reaction mixture was added ethereal solution of trifluoroborane (3 eq) at 0° C. and stirred for 14-16 hours at ambient temperature. To it was added sodium hydroxide (1N) solution at 0° C. and stirred for 1 hour. The reaction mixture was diluted with ethylacetate and water. The combined extract was washed with saturated solution of sodium chloride and concentrated under reduced pressure. The residue thus obtained was purified by column chromatography to furnish the title compound. Yield: 0.340 g

### Step f: Synthesis of 3-[3,4-bis(benzyloxy)phenyl]-1,7-dioxa-2-azaspiro[4.4]non-2-ene

To a solution of the compound obtained from step e above (1 eq) in tetrahydrofuran, triphenylphosphine (1.12 eq) and succinimide (1 eq) was added diisopropyldiazadicarboxylate (1.14 eq). The reaction mixture was stirred at room temperature for overnight. The organic solvent was removed under reduced pressure and the residue thus obtained was purified by column chromatography to furnish the title compound. Yield: 250 mg.

### Step g: Synthesis of 4-(1,7-dioxa-2-azaspiro[4.4]non-2-en-3-yl)benzene-1,2-diol

### Compound No. 34

To a solution of the compound obtained from step f above (00.250 g, 0.6 mmole) in methanol (10 ml), was added palladium on carbon (0.500 g, 10%). The reaction mixture was evacuated with hydrogen gas and the resulting reaction mixture was allowed to stir under hydrogen atmosphere at room temperature for 1 hour. The reaction mixture was filtered through celite pad. The filtrate was concentrated under reduced pressure to furnish the title compound. Yield: 110 mg. Mass (m/z): 236.19 (M⁺+1).

### Example 8

### Synthesis of 3-(2,3-Dihydro-1,4-benzodioxin-6-yl)-1,7-dioxa-2-azaspiro[4.4]non-2-ene

### Compound No. 51

To a solution of Compound No. 34 (0.200 g, 0.85 mmol) above in dimethylformamide (60 ml), was added 1,2-dibromoethane (0.160 g, 0.85 mmol) and potassium carbonate (0.176 g, 1.27 mmol). The reaction mixture was stirred for 20 hours at 60-65° C. The mixture was extracted with ethyl acetate, washed with brine and water and concentrated under reduced pressure. The residue thus obtained was purified by column chromatography using 20% ethyl acetate in hexane solvent mixture as eluent to furnish the title compound. Yield: 0.079 gm. Mass (m/z): 262.17 (M⁺+1).

### Example 9

### 3-[3,4-bis(cyclopentyloxy)phenyl]-1,7-dioxa-2-azaspiro[4.4]non-2-ene

### Compound No. 27

To a solution of Compound No. 34 (0.070 g, 0.29 mmol) in dimethylformamide (2 ml), was added potassium carbonate (0.164 g, 1.1 mmol) and cyclopentyl bromide (0.132 g, 0.891 mmol). The reaction mixture was stirred for 20 hours at 50-60° C. The mixture was extracted with ethyl acetate, washed with water, dried over anhydrous sodium sulphate and concentrated under reduced pressure. The residue thus obtained was purified by column chromatography by using 20% ethyl acetate in hexane solvent mixture as eluent to furnish the title compound. Yield: 0.040 gm. Mass (m/z): 372.14 (M⁺+1).

Analogues of 3-[3,4-bis(cyclopentyloxy)phenyl]-1,7-dioxa-2-azaspiro[4.4]non-2-ene (Compound No. 27) described below, can be prepared analogously,
3-[3,4-bis(2-morpholin-4-ylethoxy)phenyl]-1,7-dioxa-2-azaspiro[4.4]non-2-ene (Compound No. 12)

Mass (m/z): 349.19 (M⁺+1),
3-(3,4-diisopropoxyphenyl)-1,7-dioxa-2-azaspiro[4.4]non-2-ene (Compound No. 13)

Mass (m/z): 320.21 (M⁺+1),
3-[3,4-Bis(cyclopropylmethoxy)phenyl]-1,7-dioxa-2-azaspiro[4.4]non-2-ene (Compound No. 28)

Mass (m/z): 344.12 (M⁺+1),
3-[3,4-Bis(benzyloxy)phenyl]-1,7-dioxa-2-azaspiro[4.4]non-2-ene (Compound No. 33)

Mass (m/z): 416.06 (M⁺+1).

### Example 10

### 2-(Cyclopentyloxy)-4-(1,7-dioxa-2-azaspiro[4.4]non-2-en-3-yl)phenol

### Compound No. 62

To a solution of the compound 3-[3-(cyclopentyloxy)-4-methoxyphenyl]-1,7-dioxa-2-azaspiro[4.4]non-2-ene (disclosed in our copending patent application U.S. Ser. No. 60/498,947) (100 mg, 0.315 mmol) in dimethylacetamide (2 ml), sodium ethane thiolate (79.6 mg, 0.94637 mmol) and stirred the reaction mixture at 110° C. for 7-9 hours under nitrogen atmosphere. The mixture was quenched with aqueous ammonium chloride and extracted with ethyl acetate. The organic layer was washed with water, dried over anhydrous sodium sulphate and concentrated under reduced pressure to furnish the title compound. Yield: 90 mg. Mass (m/z): 304.23 (M⁺+1).

Analogues of 2-(cyclopentyloxy)-4-(1,7-dioxa-2-azaspiro[4.4]non-2-en-3-yl)phenol (Compound No. 62) described below can be prepared analogously,
2-(2,3-Dihydro-1H-inden-2-yloxy)-4-(1,7-dioxa-2-azaspiro[4.4]non-2-en-3-yl)phenol (Compound No. 161)

Mass (m/z): 352.0 (M⁺+1).

### Example 11

### 3-[3-(Cyclopentyloxy)-4-ethoxyphenyl]-1,7-dioxa-2-azaspiro[4.4]non-2-ene

### Compound No. 85

To a solution of the Compound No. 62 (50 mg, 0.16 mmole) in dimethylformamide (2 ml), was added potassium carbonate (46 mg, 0.33 mmole) and heated the reaction mixture to 60° C. To the resulting mixture was added ethyl bromide (36 mg, 0.33 mmole) dropwise and stirred the reaction mixture at 60° C. for 10 hours. The reaction mixture was diluted with water and extracted with ethyl acetate. The organic extracts were collected, washed with brine, dried over anhydrous sodium sulphate and concentrated under reduced pressure. The residue thus obtained was purified by column chromatography to furnish the title compound. Yield: 46 mg. Mass (m/z): 332.18 (M⁺+1).

Analogues of 3-[3-(Cyclopentyloxy)-4-ethoxyphenyl]-1,7-dioxa-2-azaspiro[4.4]non-2-ene (Compound no. 85) described below can be prepared similarily,
3-[3-methoxy-4-(2-morpholin-4-ylethoxy)phenyl]-1,7-dioxa-2-azaspiro[4.4]non-2-ene (Compound No. 14)

Mass (m/z): 363.24 (M⁺+1),
3-(4-Butoxy-3-isobutoxyphenyl)-1,7-dioxa-2-azaspiro[4.4]non-2-ene (Compound No. 63)

Mass (m/z): 348.33 (M⁺+1),
3-(3-Isobutoxy-4-propoxyphenyl)-1,7-dioxa-2-azaspiro[4.4]non-2-ene (Compound No. 64)

Mass (m/z): 334.21 (M⁺+1),
3-[3-Butoxy-4-(cyclopropylmethoxy)phenyl]-1,7-dioxa-2-azaspiro[4.4]non-2-ene (Compound No. 65)

Mass (m/z): 346.23 (M⁺+1),
3-(3-Butoxy-4-ethoxyphenyl)-1,7-dioxa-2-azaspiro[4.4]non-2-ene (Compound No. 66)

Mass (m/z): 320.23 (M⁺+1),
3-[3-Butoxy-4-(cyclohexyloxy)phenyl]-1,7-dioxa-2-azaspiro[4.4]non-2-ene (Compound No. 67)

Mass (m/z): 388.26 (M⁺+1),
3-[3-(Cyclohexylmethoxy)-4-ethoxyphenyl]-1,7-dioxa-2-azaspiro[4.4]non-2-ene (Compound No. 68)

Mass (m/z): 360.22 (M⁺+1),
3-[3-(Cyclohexylmethoxy)-4-isopropoxyphenyl]-1,7-dioxa-2-azaspiro[4.4]non-2-ene (Compound No. 69)

Mass (m/z): 374.27 (M⁺+1),
3-[4-Butoxy-3-(cyclohexylmethoxy)phenyl]-1,7-dioxa-2-azaspiro[4.4]non-2-ene (Compound No. 70)

Mass (m/z): 388.26 (M⁺+1),
3-(4-Isobutoxy-3-isopropoxyphenyl)-1,7-dioxa-2-azaspiro[4.4]non-2-ene (Compound No. 71)

Mass (m/z): 334.28 (M⁺+1),
3-(4-Butoxy-3-isopropoxyphenyl)-1,7-dioxa-2-azaspiro[4.4]non-2-ene (Compound No. 72)

Mass (m/z): 334.21 (M⁺+1),
3-[4-(Cyclohexylmethoxy)-3-isopropoxyphenyl]-1,7-dioxa-2-azaspiro[4.4]non-2-ene (Compound No. 73)

Mass (m/z): 374.27 (M⁺+1),
3-[3-Isopropoxy-4-(2-morpholin-4-ylethoxy)phenyl]-1,7-dioxa-2-azaspiro[4.4]non-2-ene (Compound No. 74)

Mass (m/z): 391.19 (M⁺+1),
3-[3-(Cyclopropylmethoxy)-4-isopropoxyphenyl]-1,7-dioxa-2-azaspiro[4.4]non-2-ene (Compound No. 75)

Mass (m/z): 346.20 (M⁺+1),
3-[3-(Cyclopropylmethoxy)-4-(2-morpholin-4-ylethoxy)phenyl]-1,7-dioxa-2-azaspiro[4.4]non-2-ene (Compound No. 76)

Mass (m/z): 403.22 (M⁺+1),
3-[4-Butoxy-3-(cyclopropylmethoxy)phenyl]-1,7-dioxa-2-azaspiro[4.4]non-2-ene (Compound No. 77)

Mass (m/z): 346.19 (M⁺+1),
3-[3-(Cyclopropylmethoxy)-4-isopropoxyphenyl]-1,7-dioxa-2-azaspiro[4.4]non-2-ene (Compound No. 78)

Mass (m/z): 332.18 (M⁺+1),
3-(3-Isobutoxy-4-isopropoxyphenyl)-1,7-dioxa-2-azaspiro[4.4]non-2-ene (Compound No. 79)

Mass (m/z): 334.21 (M⁺+1),
3-[4-(Cyclopropylmethoxy)-3-isobutoxyphenyl]-1,7-dioxa-2-azaspiro[4.4]non-2-ene (Compound No. 80)

Mass (m/z): 346.29 (M⁺+1),
3-[4-(cyclohexyloxy)-3-(cyclopentyloxy)phenyl]-1,7-dioxa-2-azaspiro[4.4]non-2-ene (Compound No. 81)

Mass (m/z): 386.23 (M⁺+1),
3-[4-(Cyclohexylmethoxy)-3-(cyclopentyloxy)phenyl]-1,7-dioxa-2-azaspiro[4.4]non-2-ene (Compound No. 82)

Mass (m/z): 400.21 (M⁺+1),
3-[4-(Cyclopropylmethoxy)-3-(cyclopentyloxy)phenyl]-1,7-dioxa-2-azaspiro[4.4]non-2-ene (Compound No. 83)

Mass (m/z): 358.19 (M⁺+1),
3-[3-(Cyclopentyloxy)-4-isobutoxyphenyl]-1,7-dioxa-2-azaspiro[4.4]non-2-ene (Compound No. 84)

Mass (m/z): 360.22 (M⁺+1),
3-[3-(cyclopropylmethoxy)-4-ethoxyphenyl]-1,7-dioxa-2-azaspiro[4.4]non-2-ene (Compound No. 86)

Mass (m/z): 318.20 (M⁺+1),
3-[4-(Cyclopentyloxy)-3-isobutoxyphenyl]-1,7-dioxa-2-azaspiro[4.4]non-2-ene (Compound No. 87)

Mass (m/z): 360.21 (M⁺+1),
3-[3-Isopropoxy-4-(2-morpholin-4-ylethoxy)phenyl]-1,7-dioxa-2-azaspiro[4.4]non-2-ene (Compound No. 88)

Mass (m/z): 405.18 (M⁺+1),
3-(4-Ethoxy-3-isobutoxyphenyl)-1,7-dioxa-2-azaspiro[4.4]non-2-ene (Compound No. 89)

Mass (m/z): 320.16 (M⁺+1),
3-[3-(Cyclopentyloxy)-4-propoxyphenyl]-1,7-dioxa-2-azaspiro[4.4]non-2-ene (Compound No. 90)

Mass (m/z): 346.16 (M⁺+1),
3-[4-Butoxy-3-(cyclopentyloxy)phenyl]-1,7-dioxa-2-azaspiro[4.4]non-2-ene (Compound No. 91)

Mass (m/z): 360.21 (M⁺+1),
3-[3-(Cyclopentyloxy)-4-isopropoxyphenyl]-1,7-dioxa-2-azaspiro[4.4]non-2-ene (Compound No. 92)

Mass (m/z): 346.16 (M⁺+1),
3-[3-(Cyclopentyloxy)-4-(cycloheptyloxy)phenyl]-1,7-dioxa-2-azaspiro[4.4]non-2-ene (Compound No. 93)

Mass (m/z): 400.21 (M⁺+1),
3-[3-(Cyclopentyloxy)-4-(2-morpholin-4-ylethoxy)phenyl]-1,7-dioxa-2-azaspiro[4.4]non-2-ene (Compound No. 94)

Mass (m/z): 417.21 (M⁺+1),
3-[4-(Cyclohexylmethoxy)-3-isobutoxyphenyl]-1,7-dioxa-2-azaspiro[4.4]non-2-ene (Compound No. 95)

Mass (m/z): 388.19 (M⁺+1),
3-[4-(Cyclohexylmethoxy)-3-(cyclopropylmethoxy)phenyl]-1,7-dioxa-2-azaspiro[4.4]non-2-ene (Compound No. 96)

Mass (m/z): 386.23 (M⁺+1),
3-[3-(Cyclopropylmethoxy)-4-propoxyphenyl]-1,7-dioxa-2-azaspiro[4.4]non-2-ene (Compound No. 97)

Mass (m/z): 332.25 (M⁺+1),
3-[4-(Cyclopentyloxy)-3-(cyclopropylmethoxy)phenyl]-1,7-dioxa-2-azaspiro[4.4]non-2-ene (Compound No. 98)

Mass (m/z): 358.19 (M⁺+1),
3-[4-(Cyclopropylmethoxy)-3-isopropoxyphenyl]-1,7-dioxa-2-azaspiro[4.4]non-2-ene (Compound No. 99)

Mass (m/z): 332.25 (M⁺+1),
3-[4-(Cyclopentyloxy)-3-isopropoxyphenyl]-1,7-dioxa-2-azaspiro[4.4]non-2-ene (Compound No. 100)

Mass (m/z): 346.23 (M⁺+1),
3-(3-Isopropoxy-4-propoxyphenyl)-1,7-dioxa-2-azaspiro[4.4]non-2-ene (Compound No. 101)

Mass (m/z): 320.23 (M⁺+1),
3-(4-Ethoxy-3-isopropoxyphenyl)-1,7-dioxa-2-azaspiro[4.4]non-2-ene (Compound No. 102)

Mass (m/z): 306.25 (M⁺+1),
3-[3-Butoxy-4-(2-morpholin-4-ylethoxy)phenyl]-1,7-dioxa-2-azaspiro[4.4]non-2-ene (Compound No. 103)

Mass (m/z): 405.18 (M⁺+1),
3-[3-Butoxy-4-(cyclopentyloxy)phenyl]-1,7-dioxa-2-azaspiro[4.4]non-2-ene (Compound No. 104)

Mass (m/z): 360.24 (M⁺+1),
3-(3-Butoxy-4-propoxyphenyl)-1,7-dioxa-2-azaspiro[4.4]non-2-ene (Compound No. 105)

Mass (m/z): 334.21 (M⁺+1),
3-(3-Butoxy-4-isopropoxyphenyl)-1,7-dioxa-2-azaspiro[4.4]non-2-ene (Compound No. 106)

Mass (m/z): 334.21 (M⁺+1),
3-[3-(Cyclohexylmethoxy)-4-propoxyphenyl]-1,7-dioxa-2-azaspiro[4.4]non-2-ene (Compound No. 107)

Mass (m/z): 374.27 (M⁺+1),
3-[3-(Cyclohexylmethoxy)-4-isobutoxyphenyl]-1,7-dioxa-2-azaspiro[4.4]non-2-ene (Compound No. 108)

Mass (m/z): 388.19 (M⁺+1),
3-[3-(Cyclohexylmethoxy)-4-(cyclopentyloxy)phenyl]-1,7-dioxa-2-azaspiro[4.4]non-2-ene (Compound No. 109)

Mass (m/z): 400.21 (M⁺+1),
3-[3-(Cyclohexylmethoxy)-4-(cyclopropylmethoxy)phenyl]-1,7-dioxa-2-azaspiro[4.4]non-2-ene (Compound No. 110)

Mass (m/z): 386.23 (M⁺+1),
3-[4-(Cyclohexylmethoxy)-3-propoxyphenyl]-1,7-dioxa-2-azaspiro[4.4]non-2-ene (Compound No. 111)

Mass (m/z): 374.27 (M⁺+1),
3-[4-(Cyclopropylmethoxy)-3-propoxyphenyl]-1,7-dioxa-2-azaspiro[4.4]non-2-ene (Compound No. 112)

Mass (m/z): 332.18 (M⁺+1),
3-[4-(Cyclopentyloxy)-3-propoxyphenyl]-1,7-dioxa-2-azaspiro[4.4]non-2-ene (Compound No. 113)

Mass (m/z): 346.23 (M⁺+1),
3-[4-Isobutoxy)-3-propoxyphenyl]-1,7-dioxa-2-azaspiro[4.4]non-2-ene (Compound No. 114)

Mass (m/z): (M⁺+1),
3-[3-(Cycloheptyloxy)-4-(cyclopropylmethoxy)phenyl]-1,7-dioxa-2-azaspiro[4.4]non-2-ene (Compound No. 115)

Mass (m/z): 386.23 (M⁺+1),
3-[3-(Cycloheptyloxy)-4-propoxyphenyl]-1,7-dioxa-2-azaspiro[4.4]non-2-ene (Compound No. 116)

Mass (m/z): 374.27 (M⁺+1),
3-[4-Butoxy-3-(cycloheptyloxy)phenyl]-1,7-dioxa-2-azaspiro[4.4]non-2-ene (Compound No. 117)

Mass (m/z): 388.26 (M⁺+1),
3-[3-(Cycloheptyloxy)-4-isopropoxyphenyl]-1,7-dioxa-2-azaspiro[4.4]non-2-ene (Compound No. 118)

Mass (m/z): 374.08 (M⁺+1),
3-[3-(Cycloheptyloxy)-4-(cyclopentyloxy)phenyl]-1,7-dioxa-2-azaspiro[4.4]non-2-ene (Compound No. 119)

Mass (m/z): 428.26 (M⁺+1),
3-(3-Ethoxy-4-propoxyphenyl)-1,7-dioxa-2-azaspiro[4.4]non-2-ene (Compound No. 120)

Mass (m/z): 306.18 (M⁺+1),
3-[4-(Cycloheptyloxy)-3-ethoxyphenyl]-1,7-dioxa-2-azaspiro[4.4]non-2-ene (Compound No. 121)

Mass (m/z): 360.29 (M⁺+1),
3-[4-(Cyclopropylmethoxy)-3-ethoxyphenyl]-1,7-dioxa-2-azaspiro[4.4]non-2-ene (Compound No. 122)

Mass (m/z): 318.20 (M⁺+1),
3-[4-(Cyclohexylmethoxy)-3-ethoxyphenyl]-1,7-dioxa-2-azaspiro[4.4]non-2-ene (Compound No. 123)

Mass (m/z): 360.22 (M⁺+1),
3-(3-Butoxy-4-isobutoxyphenyl)-1,7-dioxa-2-azaspiro[4.4]non-2-ene (Compound No. 125)

Mass (m/z): 348.18 (M⁺+1),
3-(3-Ethoxy-4-isopropoxyphenyl)-1,7-dioxa-2-azaspiro[4.4]non-2-ene (Compound No. 126)

Mass (m/z): 306.16 (M⁺+1),
3-[4-(Cyclopentyloxy)-3-ethoxyphenyl]-1,7-dioxa-2-azaspiro[4.4]non-2-ene (Compound No. 127)

Mass (m/z): 332.20 (M⁺+1),
3-(4-Butoxy-3-ethoxyphenyl)-1,7-dioxa-2-azaspiro[4.4]non-2-ene (Compound No. 128)

Mass (m/z): 320.18 (M⁺+1),
3-(3-Ethoxy-4-isobutoxyphenyl)-1,7-dioxa-2-azaspiro[4.4]non-2-ene (Compound No. 129)

Mass (m/z): 320.18 (M⁺+1),
3-[3-(Cycloheptyloxy)-4-isobutoxyphenyl]-1,7-dioxa-2-azaspiro[4.4]non-2-ene (Compound No. 130)

Mass (m/z): 388.20 (M⁺+1),
3-[3-(Cycloheptyloxy)-4-(cyclopentyloxy)phenyl]-1,7-dioxa-2-azaspiro[4.4]non-2-ene (Compound No. 131)

Mass (m/z): 400.22 (M⁺+1),
3-[3-(Cycloheptyloxy)-4-ethoxyphenyl]-1,7-dioxa-2-azaspiro[4.4]non-2-ene (Compound No. 132)

Mass (m/z): 360.20 (M⁺+1),
3-(4-Butoxy-3-propoxyphenyl)-1,7-dioxa-2-azaspiro[4.4]non-2-ene (Compound No. 133)

Mass (m/z): 334.21 (M⁺+1),
3-(4-Ethoxy-3-propoxyphenyl)-1,7-dioxa-2-azaspiro[4.4]non-2-ene (Compound No. 134)

Mass (m/z): 306.22 (M⁺+1),
3-[4-(Morpholin-4-ylmethoxy)-3-propoxyphenyl]-1,7-dioxa-2-azaspiro[4.4]non-2-ene (Compound No. 135)

Mass (m/z): 391.16 (M⁺+1),
3-(4-Isopropoxy-3-propoxyphenyl)-1,7-dioxa-2-azaspiro[4.4]non-2-ene (Compound No. 136)

Mass (m/z): 320.18 (M⁺+1),
3-[4-(Difluoromethoxy)-3-(2,3-dihydro-1H-inden-2-yloxy)phenyl]-1,7-dioxa-2-azaspiro[4.4]non-2-ene (Compound No. 151)

Mass (m/z): 402.0 (M⁺+1),
3-[4-(Cyclopentyloxy)-3-(2,3-dihydro-1H-inden-2-yloxy)phenyl]-1,7-dioxa-2-azaspiro[4.4]non-2-ene (Compound No. 152)

Mass (m/z): 420.10 (M⁺+1),
3-[4-Butoxy-3-(2,3-dihydro-1H-inden-2-yloxy)phenyl]-1,7-dioxa-2-azaspiro[4.4]non-2-ene (Compound No. 153)

Mass (m/z): 408.2 (M⁺+1),
3-[3-(2,3-Dihydro-1H-inden-2-yloxy)-4-ethoxyphenyl]-1,7-dioxa-2-azaspiro[4.4]non-2-ene (Compound No. 157)

Mass (m/z): 380.04 (M⁺+1),
3-[3-(2,3-Dihydro-1H-inden-2-yloxy)-4-propoxyphenyl]-1,7-dioxa-2-azaspiro[4.4]non-2-ene (Compound No. 158)

Mass (m/z): 394.08 (M⁺+1),
3-[4-(Cyclopropylmethoxy)-3-(2,3-dihydro-1H-inden-2-yloxy)phenyl]-1,7-dioxa-2-azaspiro[4.4]non-2-ene (Compound No. 159)

Mass (m/z): 406.05 (M⁺+1),
3-[3-(2,3-Dihydro-1H-inden-2-yloxy)-4-isopropoxyphenyl]-1,7-dioxa-2-azaspiro[4.4]non-2-ene (Compound No. 160)

Mass (m/z): 394.2 (M⁺+1),

### Example 12

### 3-[3-(Difluoromethoxy)-4-methoxyphenyl]-1,7-dioxa-2-azaspiro[4.4]non-2-ene

### Compound No. 40

To a solution of the compound 5-(1,7-Dioxa-2-aza-spiro[4.4]non-2-en-3-yl)-2-methoxy-phenol (disclosed in our copending patent application U.S. Ser. No. 60/498,947) (90 mg) 90 mg) in dimethylformamide (10 ml), benzyltriethyl ammonium chloride (0.036 mole) was added. To the resulting reaction mixture was added sodium hydroxide solution (0.0018 mole of 30% solution) dropwise for about 3 minutes with a continuous flow of chloro-difluoro methane. The reaction mixture was acidified with dilute hydrochloric acid and diluted with water. The reaction mixture was extracted with ethyl acetate, washed with saturated solution of sodium chloride and concentrated under reduced pressure. The residue thus obtained was purified by column chromatography to furnish the title compounds. Yield: 25 mg. Mass (m/z): 300.1. (M⁺+1).

Analogues of 3-[3-(Difluoromethoxy)-4-methoxyphenyl]-1,7-dioxa-2-azaspiro[4.4]non-2-ene (Compound No. 40), described below can prepared analogously,
3-[3-(Allyloxy)-4-methoxyphenyl]-1,7-dioxa-2-azaspiro[4.4]non-2-ene (Compound No. 60)

Mass (m/z): 290.11 (M⁺+1),
3-[3-(2-Chloroethoxy)-4-methoxyphenyl]-1,7-dioxa-2-azaspiro[4.4]non-2-ene (Compound No. 61)

Mass (m/z): 312.12 (M⁺+1),
3-[4-Methoxy-3-(pyridin-3-ylmethoxy)phenyl]-1,7-dioxa-2-azaspiro[4.4]non-2-ene (Compound No. 146)

Mass (m/z): 341.06 (M⁺+1),
3-[4-Methoxy-3-(pyridin-2-ylmethoxy)phenyl]-1,7-dioxa-2-azaspiro[4.4]non-2-ene (Compound No. 156)

Mass (m/z): 341.0 (M⁺+1),
Ethyl [5-(1,7-dioxa-2-azaspiro[4.4]non-2-en-3-yl)-2-methoxyphenoxy]acetate (Compound No. 165)

Mass (m/z): 336.0 (M⁺+1),
[5-(1,7-Dioxa-2-azaspiro[4.4]non-2-en-3-yl)-2-methoxyphenoxy]acetonitrile (Compound No. 166)

Mass (m/z): 289.0 (M⁺+1).

### Example 13

### 2-[5-(1,7-Dioxa-2-azaspiro[4.4]non-2-en-3-yl)-2-methoxyphenoxy]acetamide

### Compound No. 164

A solution of the Compound No. 165 (50 mg) in methanolic ammonia (2 ml, 4.5 N) was stirred at room temperature for 6 hrs followed by the removal of methanol under reduced pressure. Solid thus separated out was washed with hexane and dried under vacuum to furnish the title compound. Yield 30 mg. Mass (m/z): 307.0 (M⁺+1).

The following compound can be prepared analogously,
N-cyclopropyl-2-[5-(1,7-dioxa-2-azaspiro[4.4]non-2-en-3-yl)-2-methoxyphenoxy]acetamide (Compound No. 162)

Mass (m/z): 347.0 (M⁺+1).

### Example 14

### N-butyl-N′-{3-[3-(cyclopentyloxy)-4-methoxyphenyl]-1-oxa-2-azaspiro[4.5]dec-2-en-8-yl}urea

### Compound No. 22

To a solution of the compound hydrochloride salt of 3-(3-cyclopentyloxy-4-methoxy-phenyl)-1-oxa-2-aza-spiro[4.5]dec-2-en-8-ylamine (disclosed in U.S. patent application Ser. No. 10/930,569) (100 mg, 0.262 mmol) in dichloroethane (10 mL) was added triethylamine (0.0.04 ml, 0.0262 mmol) at room temperature followed by the addition of 1-isocyanatobutane dropwise (28 mg, 0.288 mmol). The reaction mixture was stirred at room temperature for 12 hours. The resulting mixture was quenched with aqueous sodium bicarbonate solution and dichloroethane was removed under reduced pressure. The mixture was extracted with ethyl acetate. The organic extracts were separated, washed with water and brine and dried over anhydrous sodium sulphate. They were also filtered and concentrated under reduced pressure. The residue thus obtained was purified by column chromatography to furnish the title compound. Yield: 60 mg. Mass (m/z): 444.23 (M⁺+1).

The following compounds can be prepared analogously,
N-{3-[3-(cyclopentyloxy)-4-methoxyphenyl]-1-oxa-2-azaspiro[4.5]dec-2-en-8-yl}-N-(2-methoxyphenyl)urea (Compound No. 23)

Mass (m/z): 494.19 (M⁺+1),
Tert-butyl [({3-[3-(cyclopentyloxy)-4-methoxyphenyl]-1-oxa-2-azaspiro[4.5]dec-2-en-8-yl}amino)carbonyl]carbamate (Compound No. 46)

Mass (m/z): 502.22 (M⁺+1),

### Example 15

### N-{3-[3-(Cyclopentyloxy)-4-methoxyphenyl]-1-oxa-2-azaspiro[4.5]dec-2-en-8-yl}cyclopentanecarboxamide

### Compound No. 47

To a solution of the compound hydrochloride salt of 3-(3-cyclopentyloxy-4-methoxy-phenyl)-1-oxa-2-aza-spiro[4.5]dec-2-en-8-ylamine (disclosed in U.S. patent application Ser. No. 10/930,569) (100 mg, 0.260 mmol) in dimethylformamide (1 mL) was added cyclopentylcarboxylic acid (0.025 ml, 0.236 mmole). The reaction mixture was cooled to 0° C. stirred followed by the addition of N-methylmorpholine (0.0318 ml, 0.289 mmol) and hydroxybenzotriazole 39 mg, 0.289 mmole). The resulting mixture was stirred for 30 minutes at the same temperature followed by the addition of 1-(3-dimethylaminopropyl)-3-ethylcarbodiimide hydrochloride (55 mg, 0.289 mmol). The mixture was again stirred for 10 hours. The resulting mixture was diluted with water and extracted with ethyl acetate. The organic extracts were separated, washed with water and brine and dried over anhydrous sodium sulphate. They were then filtered and concentrated under reduced pressure and the residue thus obtained was purified by column chromatography using 5% methanol in ethyl acetate solvent mixture as eluent to furnish the title compound. Yield: 80 mg. Mass (m/z): 441.34 (M⁺+1).

The following compounds can be prepared analogously,
N-{3-[3-(Cyclopentyloxy)-4-methoxyphenyl]-1-oxa-2-azaspiro[4.5]dec-2-en-8-yl}-2-fluorobenzamide (Compound No. 138)

Mass (m/z): 467.0 (M⁺+1),
N-{3-[3-(Cyclopentyloxy)-4-methoxyphenyl]-1-oxa-2-azaspiro[4.5]dec-2-en-8-yl}benzamide (Compound No. 139)

Mass (m/z): 449.0 (M⁺+1).

### Example 16

### N-{3-[3-(Cyclopentyloxy)-4-methoxyphenyl]-1-oxa-2-azaspiro[4.5]dec-2-en-8-yl}methanesulfonamide

### Compound No. 58

To a solution of the compound hydrochloride salt of 3-(3-cyclopentyloxy-4-methoxy-phenyl)-1-oxa-2-aza-spiro[4.5]dec-2-en-8-ylamine (disclosed in our copending patent application U.S. Ser. No. 60/498,947) (0.17 gm, 0.45 mmole) in dichloromethane (50 mL) was added triethylamine (0.13 ml, 0.090 mmole) at room temperature followed by the addition of methane sulphonylchloride (0.05 ml, 0.58 mmole). The reaction mixture was stirred at room temperature for 2 hours. The resulting mixture was quenched with aqueous sodium bicarbonate solution and extracted with ethyl acetate followed by the removal of dichloromethane under reduced pressure. The organic extracts were separated, washed with water and brine and dried over anhydrous sodium sulphate. They were then filtered and concentrated under reduced pressure to furnish the title compound. Yield: 70 mg.

### Example 17

### 3-[3-(Cyclopentyloxy)-4-methoxyphenyl]-3a,4,5,6,7,7a-hexahydro-1,2-benzisoxazole

### Compound No. 56

To a solution of the compound 3-(cyclopentyloxy)-4-methoxybenzaldehyde oxime (disclosed in our copending patent application U.S. Ser. No. 60/498,947) (0.26 g, 1.11 mmol), cyclohexene (0.091 g, 1.11 mmol), 3 to 4 drops of pyridine in 20% chloroform in dichloromethane (50 ml) was added sodium hypochlorite (4%, 2.5 ml, 1.33 mmol) under nitrogen atmosphere. The resulting reaction mixture was stirred at room temperature for 18 hours followed by the addition of aqueous sodium hypochlorite (4%, 2.5 ml, 1.33 mmol) dropwise again. The reaction mixture was again stirred for 36 hours, washed with water and brine, dried over anhydrous sodium sulphate and concentrated under reduced pressure. The residue thus obtained was purified by column chromatography to furnish the title compound. Yield: 0.100 g. Mass (m/z): 317 (M⁺+1).

The following compounds can be prepared analogously,
3-[3-(cyclopentyloxy)-4-methoxyphenyl]-1-oxa-2-azaspiro[4.4]non-2-ene (Compound No. 11)

Mass (m/z): 316.25 (M⁺+1),
Ethyl 8-benzyl-3-[3-(cyclopentyloxy)-4-methoxyphenyl]-1-oxa-2,8-diazaspiro[4.5]dec-2-ene-4-carboxylate (Compound No. 36)

Mass (m/z): 493.33 (M⁺+1),
Ethyl 3-[3-(cyclopentyloxy)-4-methoxyphenyl]-1-oxa-2-azaspiro[4.5]dec-2-ene-4-carboxylate (Compound No. 39)

Mass (m/z): 402.17 (M⁺+1),
3-[3-(Cyclopentyloxy)-4-methoxyphenyl]-3a,6a-dimethyl-3aH-cyclopenta[d]isoxazole-4,6(5H,6aH)-dione (Compound No. 43)

Mass (m/z): 406.25 (M⁺+1),
3-[3-(Cyclopentyloxy)-4-methoxyphenyl]-6,6a-dihydro furo[3,4-d]isoxazol-4(3aH)-one (Compound No. 45)

Mass (m/z): 318.34. (M⁺+1),
3-[3-(Cyclopentyloxy)-4-methoxyphenyl]-1,8-dioxa-2-azaspiro[4.5]dec-2-ene (Compound No. 52)

Mass (m/z): 332.18 (M⁺+1),
3-[3-(Cyclopentyloxy)-4-methoxyphenyl]-3aH-cyclopenta[d]isoxazole-4,6(5H,6aH)-dione (Compound No. 53)

Mass (m/z): 332.30 (M⁺+1),
3-[3-(Cyclopentyloxy)-4-methoxyphenyl]-4,5,6,6a-tetrahydro-3aH-cyclopenta[d]isoxazole (Compound No. 57)

Mass (m/z): 302.0 (M⁺+1),
Tert-butyl 3-[3-(cyclopentyloxy)-4-methoxyphenyl]-3a,4,6,6a-tetrahydro-5H-pyrrolo[3,4-d]isoxazole-5-carboxylate (Compound No. 142)

Mass (m/z): 303.16 (M⁺+BOC)
3-[3-(Cyclopentyloxy)-4-methoxyphenyl]-3a,5,6,7a-tetrahydro-1,2-benzisoxazol-7(4H)-one (Compound No. 150)

Mass (m/z): 330.10 (M⁺+1).

### Example 18

### 3-[3-(Cyclopentyloxy)-4-methoxyphenyl]-1-oxa-2-azaspiro[4.5]dec-2-ene-4-carboxylic acid

### Compound No. 37

Compound No. 39 (50 mg, 0.12 mmole) was dissolved in ethanol (1.5 mL) and lithium hydroxide in water solution (16 mg, 0.37 mmole) was added. The mixture was stirred for 4 hour at refluxing temperature. Solvent was removed under reduced pressure and the residue thus obtained was diluted with water and acidified with drop of concentrated hydrochloric acid. The organic compound was extracted with ethyl acetate, washed with brine, dried over anhydrous sodium sulphate and finally concentrated under reduced pressure to afford title organic compound with a yield of 32 mg. Mass (m/z): 374.20 (M⁺+1).

### Example 19

### 3-[3-(Cyclopentyloxy)-4-methoxyphenyl]-3a,4,6,6a-tetrahydrofuro[3,4-d]isoxazole

### Compound No. 44

### Step a: Synthesis of {3-[3-(cyclopentyloxy)-4-methoxyphenyl]-4,5-dihydroisoxazole-4,5-diyl}dimethanol

But-2-ene-1,4-diol (29 mg, 0.328 mmole) was added to the solution of the compound 3-(cyclopentyloxy)-4-methoxybenzaldehyde oxime (70 mg, 0.298 mmole) in tetrahydrofuran (10 mL), and the resulting reaction mixture was stirred at room temperature. Sodium hypochlorite (1 mL) was added slowly to the mixture thus obtained over the period of 20 minutes and the reaction mixture was allowed to stir at room temperature overnight. A second lot of sodium hypochlorite (1 mL) was again added to it and stirred for 2 hours at room temperature. Tetrahydrofuran was evaporated off and the organic compound was extracted with ethyl acetate twice. The organic layer was concentrated to yield the title compound with a yield of 25 mg.

### Step b: Synthesis of 3-[3-(Cyclopentyloxy)-4-methoxyphenyl]-3a,4,6,6a-tetrahydrofuro[3,4-d]isoxazole

A solution of the compound obtained from step a above (100 mg, 0.00031 mole) in acetic anhydride (10 ml) was refluxed for 100-110 C for 12 hours. The reaction mixture was diluted with water and extracted with ethyl acetate, dried over anhydrous sodium sulphate and concentrated under reduced pressure. The residue thus obtained was purified by column chromatography using 10% ethyl acetate in hexane solvent mixture as eluent to furnish the title compound. Yield: 65 mg. Mass (m/z): 304.38 (M⁺+1).

### Example 20

### 3-[3-(cyclopentyloxy)-4-methoxyphenyl]-1,7-dioxa-2-azaspiro[4.4]non-2-en-8-one

### Compound No. 15

To a suspension of chromic anhydride (3.6 g, 35.82 mmol) in dichloromethane (20 ml) was added pyridine (5.66 g, 71.64 mmol) and stirred the reaction mixture for 15 minutes at room temperature. To it was added a solution of the compound 2-[3-[3-(cyclopentyloxy)-4-methoxyphenyl]-5-(hydroxymethyl)-4,5-dihydroisoxazol-5-yl]ethanol (disclosed in our copending patent application U.S. Ser. No. 60/498,947) (1.0 g, 2.99 mmol) in dichloromethane (5 ml) and stirred the reaction mixture for 1 hour. The solvent was evaporated under reduced pressure and the mixture was filtered through celite pad. The filterate was concentrated under reduced pressure and the residue thus obtained was purified by column chromatography to furnish the title compound. Yield: 230 mg. Mass (m/z): 332.17 (M⁺+1).

### Example 21

### 3-[3-(cyclopentyloxy)-4-methoxyphenyl]-1,7-dioxa-2-azaspiro[4.4]non-2-en-8-ol

### Compound No. 16

A solution of the Compound No. 15 (30 mg, 0.09 mmol) in dry toluene (5 ml) was cooled to −78° C. followed by the addition of diisobutylaluminium hydride (19.3 mg, 0.14 mmol) dropwise and stirred the reaction mixture at same temperature for 2 hours under argon atmosphere. To it was added sodium potassium tartarate solution followed by ethyl acetate and water. The organic layer was separated, washed with brine and water, dried over anhydrous sodium sulphate and concentrated under reduced pressure to furnish the title compound. Yield: 18 mg. Mass (m/z): 334 (M⁺+1).

### Example 22

### 3-[3-(Cyclopentyloxy)-4-methoxyphenyl]-4,5,6,6a-tetrahydro-3aH-pyrrolo[3,4-d]isoxazole

### Compound No. 140

To a solution of the Compound No. 142 (120 mg) in dichloromethane (5 ml) at 0° C. was added methanolic hydrochloric acid (1 ml) dropwise and stirred the reaction mixture for overnight. The solvent was evaporated under reduced pressure and the residue thus obtained was recrystallised with dichloromethane in hexane (20:80) solvent mixture as eluent to furnish the title compound. Yield: 100 mg. Mass (m/z): 303.99 (M⁺+1).

### Example 23

### 5-Acetyl-3-[3-(cyclopentyloxy)-4-methoxyphenyl]-4,5,6,6a-tetrahydro-3aH-pyrrolo[3,4-d]isoxazole

### Compound No. 147

The compound No. 140 (45 mg, 0.149 mmole) and acetic anhydride (18.25 mg, 0.1788 mmole) were taken in dichloromethane (6 ml) followed by the addition of catalytic amount of dimethylamino pyridine was added and stirred for overnight. The resulting reaction mixture was diluted with water (15 ml) and extracted with dichloromethane. The organic layer was separated, washed with brine and water, dried over anhydrous sodium sulphate and concentrated under reduced pressure. The residue thus obtained was purified by column chromatography to furnish the title compound. Yield 36 mg. Mass (m/z): 345.0 (M⁺+1).

### Example 24

### 3-[3-(Cyclopentyloxy)-4-methoxyphenyl]-5-(methylsulfonyl)-4,5,6,6a-tetrahydro-3aH-pyrrolo[3,4-d]isoxazole

### Compound No. 148

The title compound was prepared following the procedure as described for the synthesis in Example 4, by using Compound No. 140 in place of hydrochloride salt of 3-[3-(cyclopentyloxy)-4-methoxyphenyl]-1-oxa-2,7-diazaspiro[4.4]non-2-ene. Yield: 35 mg.

Mass (m/z): 381.37 (M⁺+1).

### Example 25

### 3-[3-(cyclopentyloxy)-4-methoxyphenyl]-1,7-dioxa-2-azaspiro[4.4]non-2-en-6-ol

### Compound No. 1

The title compound was prepared by following the procedure as described for the synthesis of Compound No. 16, by using compound 3-[3-cyclopentyloxy-4-methoxy-phenyl)-1,7-dioxa-2-aza-spiro[4.4]non-2-ene-6-one (disclosed in our copending patent application U.S. Ser. No. 60/498,947) in place of using Compound No. 15. Yield: 28 mg.

Mass (m/z): 334.0 (M⁺+1).

### Example 26

### 3-[3-(Cyclopentyloxy)-4-methoxyphenyl]-1-oxa-2,7-diazaspiro[4.4]non-2-en-6-one

### Compound No. 42

### Step a: Synthesis of 3-[3-(cyclopentyloxy)-4-methoxyphenyl]-5-(2-hydroxyethyl)-4,5-dihydroisoxazole-5-carboxamide

To a compound 3-[3-cyclopentyloxy-4-methoxy-phenyl)-1,7-dioxa-2-aza-spiro[4.4]non-2-ene-6-one (described in copending U.S. patent application Ser. No. 10/930,569) (0.20 g) was added methanolic ammonia (3 mL) and stirred the reaction mixture for 2.5 hours at room temperature. The reaction mixture was concentrated under vacuum to yield white solid compound. Yield 0.16 gm.

### Step b: Synthesis of 2-{5-(aminocarbonyl)-3-[3-(cyclopentyloxy)-4-methoxyphenyl]-4,5-dihydroisoxazol-5-yl}ethyl methanesulfonate

The title compound was prepared following the procedure as described for the synthesis of Compound No. 4, by using the compound obtained from step a above in place of hydrochloride salt of 3-[3-(cyclopentyloxy)-4-methoxyphenyl]-1-oxa-2,7-diazaspiro[4.4]non-2-ene.

### Step c: Synthesis of 3-[3-(Cyclopentyloxy)-4-methoxyphenyl]-1-oxa-2,7-diazaspiro[4.4]non-2-en-6-one

### Compound No. 42

The compound obtained from step b above (0.16 gm, 0.375 mmole) was taken in dimethylformamide (1.4 ml) followed by the addition of anhydrous potassium carbonate (0.518 gm, 3.75 mmole) stirred for 24 hrs. The resulting reaction mixture was diluted with water and extracted with ethylacetate. Organic layer was dried over anhydrous sodium sulphate and concentrated under reduced pressure. The residue thus obtained was purified by column chromatography to give 20 mg of final product. Mass (m/z): 331.24 (M⁺+1).

### Example 27

### 7-Amino-3-[3-(cyclopentyloxy)-4-methoxyphenyl]-1-oxa-2,7-diazaspiro[4.4]non-2-en-6-one

### Compound No. 35

To a solution of the compound 3-[3-cyclopentyloxy-4-methoxy-phenyl)-1,7-dioxa-2-aza-spiro[4.4]non-2-ene-6-one (disclosed in our copending patent application U.S. Ser. No. 60/498,947) (100 mg, 0.0003 mmole) in ethanol (5 ml) was added hydrazine hydrate (0.061 ml, 0.0012 mmole) was added and refluxed for 10 hrs. Solvent was removed under reduced pressure, water was added and extracted with ethyl acetate. Organic layer was dried over anhydrous sodium sulphate and concentrated under reduced pressure. The residue thus obtained was purified by column chromatography to furnish the title compound. Yield (20 mg). Mass (m/z): 346.24 (M⁺+1).

### Example 28

### 3-[3-(cyclopentyloxy)-4-methoxyphenyl]-1-oxa-2-azaspiro[4.5]dec-2-en-8-one

### Compound No. 26

### Step a: Synthesis of 8-methylene-1,4-dioxaspiro[4.5]decane

A solution of the compound methyltriphenylphosphine iodide (19.5 g, 48.0 mmol) and potassium tert-butoxide (4.32 g, 38.4 mmol) in tetrahydrofuran (100 ml) was stirred for 3 hours at room temperature. To the resulting reaction mixture was added to a solution of 1,4-dioxaspiro[4.5]decan-8-one (3.0 g, 19.2 mmol) in tetrahydrofuran (50 ml) and stirred the mixture for 6 hours. The reaction mixture was quenched with aqueous ammonium chloride solution (10 ml) and concentrated under reduced pressure followed by diluting it with dichloromethane. The organic layer was washed with water and brine, dried over anhydrous sodium sulphate and concentrated under reduced pressure to furnish the title compound. Yield: 1.52 g.

### Step b: Synthesis of 3-[3-(cyclopentyloxy)-4-methoxyphenyl]-1,9,12-trioxa-2-azadispiro[4.2.4.2]tetradec-2-ene

The title compound was prepared following the procedure as described for the synthesis of Compound No. 21 by using the compound obtained from step a above in place of 3-methylene-piperidine-1-carboxylic acid tert-butyl ester. Yield: 0.76 g.

### Step c: Synthesis of 3-[3-(cyclopentyloxy)-4-methoxyphenyl]-1-oxa-2-azaspiro[4.5]dec-2-en-8-one

### Compound No. 26

To a solution of the compound obtained from step b above (0.6 g, 1.55 mmol) in dichloromethane (30 ml) was added trifluoroacetic acid (0.72 ml) in three lots over a time interval of 1 hour followed by the addition of water (1 ml) and stirred the reaction mixture for 6 hours at room temperature. The reaction mixture was diluted with water and extracted with dichloromethane. The organic layer was washed with aqueous sodium bicarbonate, water and brine, dried over anhydrous sodium sulphate and concentrated under reduced pressure. The residue thus obtained as purified by column chromatography to furnish the title compound. Yield: 0.44 g. Mass (m/z): 344 (M⁺+1).

### Example 29

### Synthesis of 3-[3-(cyclopentyloxy)-4-methoxyphenyl]-1-oxa-2-azaspiro[4.5]dec-2-en-8-ol

### Compound No. 24

To a solution of the Compound No. 26 (290 mg, 0.85 mmol) in methanol (50 ml) at 0° C. was added sodium borohydride (45 mg, 1.18 mmol) and stirred the reaction mixture for 2 hours. The mixture was quenched with saturated ammonium chloride and evaporated under reduced pressure. The residue thus obtained was diluted with dichloromethane, washed with water and brine, dried over anhydrous sodium sulphate and concentrated under reduced pressure. The residue thus obtained was purified by column chromatography to furnish the title compound. Yield: 0.18 g. Mass (m/z): 346 (M⁺+1).

### Example 30

### Synthesis of 3-[3-(cyclopentyloxy)-4-methoxyphenyl]-8-methyl-1-oxa-2-azaspiro[4.5]dec-2-en-8-ol

### Compound No. 59

To a solution of the Compound No. 26 (0.3 g, 0.88 mmol) in dry tetrahydrofuran (50 ml) at 0° C. was added methyl magnesium chloride (0.5 ml, 1.14 mmol) and stirred the reaction mixture for 2 hours. The mixture was quenched with aqueous ammonium hydroxide (5 ml) and concentrated under reduced pressure. The residue thus obtained was dissolved in dichloromethane, washed with water and brine, dried over anhydrous sodium sulphate and concentrated under reduced pressure. The residue thus obtained was purified by column chromatography to furnish the title compound. Yield: 0.22 g. Mass (m/z): 361 (M⁺+1).

The following compound can be prepared analogously,
3-[3-(Cyclopentyloxy)-4-methoxyphenyl]-8-vinyl-1-oxa-2-azaspiro[4.5]dec-2-en-8-ol (Compound No. 55)

Mass (m/z): 372 (M⁺+1).

### Scheme VIII, Procedure:

### Example 31

### 2-[5-(1,7-Dioxa-2-azaspiro[4.4]non-2-en-3-yl)-2-methoxyphenoxy]cyclopentanol

### Compound No. 137

To a solution of the compound 5-(1,7-dioxa-2-aza-spiro[4.4]non-2-en-3-yl)-2-methoxy-phenol (disclosed in our copending patent application U.S. Ser. No. 60/498,947) (0.11 g, 0.44 mmol) in dry dimethylformamide (20 ml) was added potassium carbonate (0.18 g, 1.33 mmol) at room temperature under nitrogen atmosphere followed by the addition of cyclopentene oxide (0.77 ml, 8.84 mmol) and stirred the reaction mixture at 80-90° C. for 24-48 hours. The reaction mixture was then diluted with ice-cold water and extracted with ethyl acetate. The combined organic extracts were washed with ice-cold water and brine, dried over anhydrous sodium sulphate and concentrated under reduced pressure. The residue thus obtained was purified by column chromatography to furnish the title compound. Yield: 0.03 g. Mass (m/z): 334.24 (M⁺+1).

### Example 32

### 3-(3-{[3-(Benzyloxy)cyclopentyl]oxy}-4-methoxyphenyl)-1,7-dioxa-2-azaspiro[4.4]non-2-ene

### Compound No. 154

The title compound was synthesised by following the procedure as described for the synthesis of (Compound No. 137) by using the compound 3-(benzyloxy)cyclopentyl methanesulfonate in place of cyclopentene oxide. Mass (m/z): 424.07 (M⁺+1).

The following compound was prepared analogously,
Hydrochloride salt of 3-[4-methoxy-3-(piperidin-3-yloxy)phenyl]-1,7-dioxa-2-azaspiro[4.4]non-2-ene (Compound No. 163)

Mass (m/z): 331.1 (M⁺−HCl).
3-{3-[(2,6-Dichloropyridin-4-yl)methoxy]-4-methoxyphenyl}-1,7-dioxa-2-azaspiro[4.4]non-2-ene (Compound No. 167)

Mass (m/z): 408.8 (M⁺+1).

### Example 33

### 2-(Difluoromethoxy)-5-(1,7-dioxa-2-azaspiro[4.4]non-2-en-3-yl)phenol

### Compound No. 41

### Step a: Synthesis of 3-(benzyloxy)-4-(difluoromethoxy)benzaldehyde oxime

Hydroxylamine hydrochloride (1.50 g, 21.58 mmole) and sodium acetate (1.769 g, 21.573 mmole) was added to a stirred solution of compound 4-(difluoromethoxy)-3-phenoxybenzaldehyde (1.50 g, 5.395 mmole) in ethanol (10 mL). The reaction mixture was stirred at room temperature for 3-4 hrs. Ethanol was evaporated under reduced pressure, which was diluted with water (20 mL) and the organic compound was extracted with ethyl acetate (2×15 mL). The ethyl acetate layer was dried over anhydrous sodium sulphate, filtered and concentrated under reduced pressure to afford the title compound.

### Step b: Synthesis of methyl 3-[3-(benzyloxy)-4-(difluoromethoxy)phenyl]-5-(2-methoxy-2-oxoethyl)-4,5-dihydroisoxazole-5-carboxylate

Dimethyl 2-methylenesuccinate (1.078 g, 6.824 mmole) was added to the solution of compound obtained from step a above (1.00 g, 3.412 mmole) in tetrahydrofuran (5 mL), and the resulting reaction mixture was stirred at room temperature. Sodium hypochlorite (10 mL) was added slowly to the mixture thus obtained over the period of 20 minutes and the reaction mixture was allowed to stir at room temperature overnight. Tetrahydrofuran was evaporated off and the organic compound was extracted with ethyl acetate twice. The organic layer was concentrated to yield the title compound with a yield of 1.50 g.

### Step c: Synthesis of 2-[3-[3-(benzyloxy)-4-(difluoromethoxy)phenyl]-5-(hydroxymethyl)-4,5-dihydroisoxazol-5-yl]ethanol

The compound obtained from step b above (1.5 g, 3.340 mmole) was dissolved in tetrahydrofuran (10 mL) and lithium hydroxide in water solution (0.68 mL of 0.5 M aqueous solution, 16.682 mmoles, 5 eq) was added. The mixture was stirred for 1 hour at room temperature. The mixture was stirred for 5 hrs at 55-60° C. Solvent was removed under reduced pressure and the residue thus obtained was diluted with water and acidified with drops of concentrated hydrochloric acid. The organic compound was extracted with ethyl acetate, washed with brine, dried over anhydrous sodium sulphate and finally concentrated under reduced pressure to afford title organic compound with a yield of 1.103 g.

### Step d: Synthesis of 2-[3-[3-(benzyloxy)-4-(difluoromethoxy)phenyl]-5-(hydroxymethyl)-4,5-dihydroisoxazol-5-yl]ethanol

The compound obtained from step c (1.1 g, 2.428 mmole) was taken in tetrahydrofuran (7 ml) followed by the addition of sodium borohydride (0.276 g, 7.26 mmole) at 0-5° C. and boron trifluoride etherate (1.02 g, 7.28 mmole) was added dropwise and stirred for 14 hrs at room temperature. Solvent was removed under reduced pressure, water was added and extracted with ethylacetate. The organic layer was dried over anhydrous sodium sulphate and concentrated under reduced pressure. The residue thus obtained was purified by column chromatography to furnish final product with the yield 0.732 g.

### Step e: Synthesis of 3-[3-(benzyloxy)-4-(difluoromethoxy)phenyl]-1,7-dioxa-2-azaspiro[4.4]non-2-ene

To a solution of the compound obtained from step d above (1 eq) in tetrahydrofuran, triphenylphosphine (1.12 eq) and succinimide (1 eq), was added diisopropyldiazadicarboxylate (1.14 eq). The reaction mixture was stirred at room temperature for overnight. The organic solvent was removed under reduced pressure and the residue thus obtained was purified by column chromatography to furnish the title compound. Yield: 40%.

### Step f: Synthesis of 2-(difluoromethoxy)-5-(1,7-dioxa-2-azaspiro[4.4]non-2-en-3-yl)phenol

### Compound No. 41

To a solution of the compound obtained from step e above (0.200 g, 0.53 mmole) in methanol (10 mL), was added palladium on carbon (300 mg, 10%). The reaction mixture was evacuated with hydrogen gas and the resulting reaction mixture was allowed to stir at room temperature for 1 hour under hydrogen atmosphere. The reaction mixture was filtered through celite pad. The filtrate was concentrated under reduced pressure to furnish the title compound. Yield=60 mg. Mass (m/z): 286.03 (M⁺+1).

### Example 34

### (S)-3-[3-(cyclopentyloxy)-4-methoxyphenyl]-1,7-dioxa-2-azaspiro[4.4]non-2-ene

### Compound No. 124

### Step a: Synthesis of L-Ephedrine salt of 5-(carboxymethyl)-3-[3-(cyclopentyloxy)-4-methoxyphenyl]-4,5-dihydroisoxazole-5-carboxylic acid

5-(carboxymethyl)-3-[3-(cyclopentyloxy)-4-methoxyphenyl]-4,5-dihydroisoxazole-5-carboxylic acid (disclosed in our copending patent application U.S. Ser. No. 60/498,947) (1.0 g, 2.87 mmol) and L-Ephedrine (0.95 g, 5.73 mmol) were dissolved in acetone (50 ml) and the mixture was refluxed for 4 h. The reaction mixture was slowly brought to room temperature (35° C.) and kept as it is for 24-36 hours to furnish the S-isomer. Yield: 0.3 g.

### Step b: Preparation of (S)-methyl 3-[3-(cyclopentyloxy)-4-methoxyphenyl]-5-(2-methoxy-2-oxoethyl)-4,5-dihydroisoxazole-5-carboxylate

Thionyl chloride (0.80 ml, 11.1 mmol) was added slowly to a dry-methanol (50 mL) at 0° C. under nitrogen atmosphere and stirred for 1 hour followed by the addition of solution of the compound obtained from step a above (1.88 g, 2.77 mmol) in dry-methanol (50 mL) at 0° C. The reaction mixture was slowly brought to room temperature and stirred at that temperature for 12 hours. The reaction mixture was concentrated and diluted with dichloromethane. The organic portion was washed with water, brine and dried over sodium sulphate and concentrated under reduced pressure. The residue thus obtained was purified by column chromatography to furnish the title compound. Yield: 0.92 g. m.p.: 92-93° C.; [α]_{D}=−113.9° (C, 1.17, CH₃OH).

¹H NMR (CDCl₃) δ 7.35 (s, 1H), 7.05 (d, J=0.02 Hz, 1H), 6.85 (d, J=0.02 Hz, 1H), 4.81 (m, 1H), 4.00 (d, J=0.04 Hz, 1H), 3.88 (s, 3H), 3.82 (s, 3H), 3.72 (s, 3H), 3.48 (d, J=0.04 Hz, 1H), 3.27 (d, J=0.04 Hz, 1H), 3.00 (d, J=0.04 Hz, 1H), 1.95 (m, 2H), 1.88 (m, 4H), 1.63 (m, 2H). Mass (m/z): 393 (M⁺+1).

### Step c: Synthesis of (S)-2-[3-[3-(cyclopentyloxy)-4-methoxyphenyl]-5-(hydroxymethyl)-4,5-dihydroisoxazol-5-yl]ethanol

The compound obtained from step b above (0.85 g, 2.17 mmol) was dissolved in tetrahydrofuran (100 mL) and cooled to 0° C. and sodium borohydride (0.41 g, 10.9 mmol) was added portion wise. The reaction mixture was stirred for 1 hour followed by the addition of methanol (10 mL). The reaction mixture was stirred for 10 hour at room temperature. Reaction mixture was filtered and the solid thus obtained was washed with tetrahydrofuran. The organic solution was cooled to 0° C. and saturated ammonium chloride solution was added slowly over a period of 30 minutes. The reaction mixture was concentrated and diluted with ethyl acetate (100 mL). The organic portion was washed with saturated ammonium chloride solution, water and brine, dried over sodium sulphate and concentrated under reduced pressure. The residue thus obtained was purified by column chromatography to furnish the title compound. Yield: 0.5 g. m.p.: 108-109° C. [α]_{D}=−5.32° (c, 1.17, CH₃OH).

¹H NMR (CDCl₃) δ 7.33 (s, 1H), 7.04 (d, J=0.02 Hz, 1H), 6.84 (d, J=0.02 Hz, 1H), 4.81 (m, 1H), 3.92-3.83 (m, 2H), 3.85 (s, 3H), 3.72 (m, 2H), 3.41 (d, J=0.04 Hz, 1H), 3.20 (d, J=0.04 Hz, 1H), 2.40 (bs, 2H, —OH), 2.07 (m, 2H), 2.05-1.83 (m, 6H), 1.63-1.61 (m, 2H). Mass (m/z): 336 (M⁺+1).

### Step d: Synthesis of (S)-3-[3-(cyclopentyloxy)-4-methoxyphenyl]-1,7-dioxa-2-azaspiro[4.4]non-2-ene (Compound No. 124)

To a solution of the compound obtained from step c above (0.43 g, 1.28 mmol), triphenyl phosphine (0.37 g, 1.41 mmol) and succinimide (0.14 g, 1.41 mmol) was added dry tetrahydrofuran (20 mL) and stirred the reaction mixture for 20 minutes at room temperature which was subsequently cooled to 0° C. Diisopropylazodicarboxylate (0.30 mL, 1.54 mmol) was added slowly over a period of 10 minutes at 0° C. and further stirred the reaction mixture at room temperature for overnight. The reaction mixture was concentrated under reduced pressure. The residue thus obtained was purified by column chromatography to furnish the title compound. Yield: 0.28 g. m.p.: 110.5° C. [α]_{D}=+1.76° (c, 1.19, CH₃OH).

¹H NMR (CDCl₃) δ 7.37 (s, 1H), 7.00 (d, J=0.02 Hz, 1H), 6.85 (d, J=0.02 Hz, 1H), 4.82 (m, 1H), 4.10 (d, J=0.03 Hz, 1H), 4.03 (m, 2H), 3.88 (s, 3H), 3.82 (d, J=0.03 Hz, 1H), 3.37 (s, 2H), 2.06 (m, 1H), 1.97-1.62 (m, 7H), 1.61 (m, 2H); Mass (m/z): 319 (M⁺+1).

The following compound can be prepared analogously by using D-Ephidrine in place of L-Ephidrine,
(R)-3-[3-(cyclopentyloxy)-4-methoxyphenyl]-1,7-dioxa-2-azaspiro[4.4]non-2-ene (Compound No. 30)

Mass (m/z): 319 (M⁺+1).

### Example 35

### 4-Bromo-3-[3-(cyclopentyloxy)-4-methoxyphenyl]-1,7-dioxa-2-azaspiro[4.4]non-2-ene

### Compound No. 149

To a solution of the compound 3-[3-(cyclopentyloxy)-4-methoxyphenyl]-1,7-dioxa-2-azaspiro[4.4]non-2-ene (disclosed in our copending patent application U.S. Ser. No. 60/498,947) (100 mg, 0.32 mmol) in chloroform (5 ml) was added N-bromosuccinimide (84 mg, 0.47 mmol) and azobutyronitrile (10 mg, 0.06 mmol). The reaction mixture was stirred for 2 hours and subsequently diluted with water. The mixture was extracted with dichloromethane, washed with water and brine, dried over anhydrous sodium sulphate and concentrated under reduced pressure. The residue thus obtained was purified with column chromatography to furnish the title compounds. Yield: 40 mg. Mass (m/z): 395.97 (M⁺+1, Compound No. 149).

### Example 36

### 3-[3-(cyclopentyloxy)-4-methoxyphenyl]-1,7-dioxa-2-azaspiro[4,4]non-2-en-4-ol

### Compound No. 29

### Step a: Synthesis of 3-[3-(cyclopentyloxy)-4-methoxyphenyl]-1,7-dioxa-2-azaspiro[4.4]non-2-en-4-yl acetate

To a solution of the 3-[3-(cyclopentyloxy)-4-methoxyphenyl]-1,7-dioxa-2-azaspiro[4.4]non-2-ene which is disclosed in our copending patent application U.S. Ser. No. 60/498,947 (100 mg, 0.26 mmol) in dimethylformamide (5 ml), was added sodium acetate (104 mg, 1.26 mmol) and stirred the mixture at 110° C. for 14 hours. The resulting reaction mixture was diluted with water and extracted with ethyl acetate. The organic layer was separated, washed with water and brine, dried over anhydrous sodium sulphate and concentrated under reduced pressure to furnish the title compound. Yield: 110 mg.

### Step b: Synthesis of 3-[3-(cyclopentyloxy)-4-methoxyphenyl]-1,7-dioxa-2-azaspiro[4.4]non-2-en-4-ol

To a solution of the compound obtained from step a above (42 mg, 0.11 mmol) in methanol (2 ml) was added potassium carbonate (46 mg, 0.34 mmol) under argon atmosphere and stirred the reaction mixture for 30 minutes at room temperature. The mixture was diluted with water and extracted with ethyl acetate. The organic layer was separated, washed with water and brine, dried over anhydrous sodium sulphate and concentrated under reduced pressure. The residue thus obtained was purified by column chromatography to furnish the title compound. Yield: 28 mg. Mass (m/z): 334.13 (M⁺+1).

### PDE-IV Enzyme Assay

The efficacy of compounds as PDE-4 inhibitor was determined by an enzyme assay (Burnouf et al.; J. Med. Chem., 2000, 43:4850-4867). The PDE-4 enzyme source used was U937 cell cytosolic fraction prepared by sonication. The enzyme reaction was carried out, with the cytosolic fraction as the enzyme source, in the presence of cAMP (1 μM) at 30° C. in the presence or absence of NCE for 45-60 min. An aliquot of this reaction mixture was taken further for the ELISA assay to determine level of cAMP in the sample. The concentration of the cAMP in the sample directly correlates with the degree of PDE-4 enzyme inhibition. Results were expressed as percent control and the IC₅₀ values of test compounds were reported to be in the range of about μM to low fM. For example, the IC₅₀ for PDE-IV inhibition ranged from about 1 μM to about 100 fM, or from about 600 nM to about 100 fM, or from about 400 nM to about 100 fM, or from about 200 nM to about 100 fM, or from about 100 nM to about 100 fM, or from about 75 nM to about 100 fM, or from about 1 nM to about 100 fM, as compared to rolipram (about 480 nM 5 repetitions). Compound No. 119 was not tested as it was insoluble under the experimental conditions.

### Cell Based Assay for TNF-α Release

### Method of Isolation of Human Peripheral Blood Mononuclear Cells:

Human whole blood was collected in vacutainer tubes containing heparin or EDTA as an anti coagulant. The blood was diluted (1:1) in sterile phosphate buffered saline and 10 ml. was carefully layered over 5 ml Ficoll Hypaque gradient (density 1.077 g/ml) in a 15 ml conical centrifuge tube. The sample was centrifuged at 3000 rpm for 25 minutes in a swing-out rotor at room temperature. After centrifugation, interface of cells were collected, diluted at least 1:5 with PBS and washed three times by centrifugation at 2500 rpm for 10 minutes at room temperature. The cells were resuspended in serum free RPMI 1640 medium at a concentration of 2 million cells/ml. Alternatively whole blood was used.

LPS stimulation of Human PBMNC's:

PBMN cells (0.1 ml; 2 million/ml) were co-incubated with 20 μl of compound (final DMSO concentration of 0.2%) for 10 min in a flat bottom 96 well microtiter plate. Compounds were dissolved in DMSO initially and diluted in medium for a final concentration of 0.2% DMSO. LPS (1 μg/ml, final concentration) was then added at a volume of 10 μl per well. After 30 min, 20 μl of fetal calf serum (final concentration of 10%) was added to each well. Cultures were incubated overnight at 37° C. in an atmosphere of 5% CO₂ and 95% air. Supernatant were then removed and tested by ELISA for TNF-α release using a commercial kit (e.g. BD Biosciences). For whole blood, the plasma samples were diluted 1:20 for ELISA. The level of TNFα in treated wells was compared with the vehicle treated controls and inhibitory potency of compound was expressed as IC₅₀ values calculated by using Graph pad prism.

Compounds 29, 33, 39, 52, 56, 57, 60, 61, 140, 148, 151, 154, 157 and 164 exhibited IC₅₀ in the TNF assay of from about 10 μM to about 0.27 nM, or from about 200 nM to about 0.24 nM, or from about 130 nM to about 0.24 nM, or from about 12 nM to about 0.24 nM, as compared to rolipram (about 240 nM, 4 repetitions).

We claim:

1. A compound having the structure of Formula I, 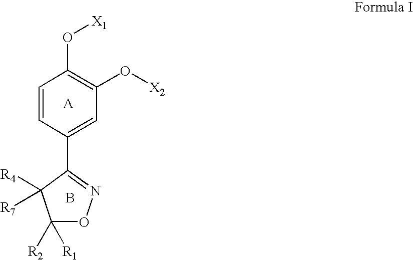
and its pharmaceutically acceptable salts, pharmaceutically acceptable solvates, enantiomers, diastereomers or N-oxides, wherein
R₁ and R₂ together forms an optionally substituted cycloalkyl or heterocyclyl ring wherein one or more optional substituent are oxo, alkyl, alkaryl, alkenyl, alkynyl, heterocyclylalkyl, cycloalkylalkyl, —SO₂NRₓR_{y}, halogen, —NH₂, —(CH₂)_{g}C(═O)NRₓR_{y}, —NHC(═O)OR₆, —NHC(═O)NRₓR_{y}, —C(═O)OR₃, —NHC(═O)Rₓ, —SO₂R₃, cyano, hydroxy, alkoxy, substituted amino, —C(═O)R₃;
R₄ is hydrogen; alkyl; hydroxy; halogen; carboxy;
R₇ is hydrogen; alkyl;
R₁ is independently hydrogen or alkyl and R₂ and R₄ forms an optionally substituted 4-12 membered saturated or unsaturated monocyclic or bicyclic ring system fused to ring B having 0-4 heteroatom(s) selected from the group consisting of N, O and S, wherein the substituents is one or more of oxo, alkyl, —C(═O)OR₃, —SO₂R₃, halogen, hydroxy, alkoxy, —NH₂ or substituted amino, with the proviso that R₂ and R₄ together does not form —CH₂—O—CH₂—O—CH₂—;
X₁ and X₂ is hydrogen, alkyl, cycloalkyl, alkaryl, alkenyl, cycloalkylalkyl, heteroaryl, heterocyclyl, heteroarylalkyl, heterocyclylalkyl, —(CH₂)_{g}C(═O)NRₓR_{y} or —(CH₂)_{g1}C(═O)OR₃ (wherein g is an integer from 0-3 and g₁ is an integer from 1-3);
X₁ and X₂ together can optionally form a cyclic ring fused with the ring A shown in Formula I, the ring containing 3-5 carbon atoms within the ring and having 2-3 heteroatoms selected from the group consisting of N, O and S;
wherein R₃ is alkyl, cycloalkyl or heterocyclyl;
wherein the halogen is F, Cl, Br, or I; Rₓ and R_{y} each independently is hydrogen, alkyl, C₃-C₆ alkenyl, C₃-C₆ alkynyl, carboxy, cycloalkyl, —S(O)ₘR₅, aryl, alkaryl, heteroaryl, heterocyclyl, heteroarylalkyl, and heterocyclylalkyl; m is an integer between 0-2; R₆ is alkyl, alkenyl, alkynyl, cycloalkyl, alkaryl, heteroarylalkyl or heterocyclylalkyl;
wherein R₅ is hydrogen, alkyl, alkenyl, alkynyl, aryl, cycloalkyl, alkaryl, heteroaryl, heteroarylalkyl, heterocyclyl or heterocyclylalkyl;

2. A compound which is selected from
3-[3-(cyclopentyloxy)-4-methoxyphenyl]-1,7-dioxa-2-azaspiro[4.4]non-2-en-6-ol (Compound No. 1).
3-[3-(Cyclopentyloxy)-4-methoxyphenyl]-N-(4-fluorophenyl)-1-oxa-2,7-diazaspiro[4.4]non-2-ene-7-carboxamide (Compound No. 2),
3-[3-(Cyclopentyloxy)-4-methoxyphenyl]-7-(tetrahydrofuran-3-ylcarbonyl)-1-oxa-2,7-diazaspiro[4.4]non-2-ene (Compound No. 3),
3-[3-(cyclopentyloxy)-4-methoxyphenyl]-N,N-dimethyl-1-oxa-2,7-diazaspiro[4.4]non-2-ene-7-sulfonamide (Compound No. 4),
N-butyl-3-[3-(cyclopentyloxy)-4-methoxyphenyl]-1-oxa-2,7-diazaspiro[4.4]non-2-ene-7-carboxamide (Compound No. 5),
2-{3-[3-(Cyclopentyloxy)-4-methoxyphenyl]-1-oxa-2,7-diazaspiro[4.4]non-2-en-7-yl}acetamide (Compound No. 6),
Hydrochloride salt of 3-[3-(cyclopentyloxy)-4-methoxyphenyl]-8-prolyl-1-oxa-2,8-diazaspiro[4.5]dec-2-ene (Compound No. 7),
3-[3-(Cyclopentyloxy)-4-methoxyphenyl]-8-(2-morpholin-4-yl-ethyl)-1-oxa-2,8-diazaspiro[4.5]dec-2-ene (Compound No. 8),
N-butyl-3-[3-(cyclopentyloxy)-4-methoxyphenyl]-1-oxa-2,8-diazaspiro[4.5]dec-2-ene-8-carboxamide (Compound No. 9),
3-[3-(cyclopentyloxy)-4-methoxyphenyl]-8-(methylsulfonyl)-1-oxa-2,8-diazaspiro[4.5]dec-2-ene (Compound No. 10),
3-[3-(cyclopentyloxy)-4-methoxyphenyl]-1-oxa-2-azaspiro[4.4]non-2-ene (Compound No. 11),
3-[3,4-bis(2-morpholin-4-ylethoxy)phenyl]-1,7-dioxa-2-azaspiro[4.4]non-2-ene (Compound No. 12),
3-(3,4-diisopropoxyphenyl)-1,7-dioxa-2-azaspiro[4.4]non-2-ene (Compound No. 13),
3-[3-methoxy-4-(2-morpholin-4-ylethoxy)phenyl]-1,7-dioxa-2-azaspiro[4.4]non-2-ene (Compound No. 14),
3-[3-(cyclopentyloxy)-4-methoxyphenyl]-1,7-dioxa-2-azaspiro[4.4]non-2-en-8-one (Compound no. 15),
3-[3-(cyclopentyloxy)-4-methoxyphenyl]-1,7-dioxa-2-azaspiro[4.4]non-2-en-8-ol (Compound No. 16).
3-[3-(Cyclopentyloxy)-4-methoxyphenyl]-7-isopropyl-1-oxa-2,7-diazaspiro[4.4]non-2-ene (Compound No. 17),
3-[3-(cyclopentyloxy)-4-methoxyphenyl]-7-(cyclopropylcarbonyl)-1-oxa-2,7-diazaspiro[4.4]non-2-ene (Compound No. 18),
N-benzyl-3-[3-(cyclopentyloxy)-4-methoxyphenyl]-1-oxa-2,7-diazaspiro[4.4]non-2-ene-7-carboxamide (Compound No. 19),
7-acetyl-3-[3-(cyclopentyloxy)-4-methoxyphenyl]-1-oxa-2,7-diazaspiro[4.4]non-2-ene (Compound No. 20),
Tert-butyl 3-[3-(cyclopentyloxy)-4-methoxyphenyl]-1-oxa-2,7-diazaspiro[4.5]dec-2-ene-7-carboxylate (Compound No. 21),
N-butyl-N′-{3-[3-(cyclopentyloxy)-4-methoxyphenyl]-1-oxa-2-azaspiro[4.5]dec-2-en-8-yl}urea (Compound No. 22),
N-{3-[3-(cyclopentyloxy)-4-methoxyphenyl]-1-oxa-2-azaspiro[4.5]dec-2-en-8-yl}-N′-(2-methoxyphenyl)urea (Compound No. 23),
3-[3-(cyclopentyloxy)-4-methoxyphenyl]-1-oxa-2-azaspiro[4.5]dec-2-en-8-ol (Compound No. 24),
Hydrochloride salt of 3-[3-(cyclopentyloxy)-4-methoxyphenyl]-1-oxa-2,7-diazaspiro[4.5]dec-2-ene (Compound No. 25),
3-[3-(cyclopentyloxy)-4-methoxyphenyl]-1-oxa-2-azaspiro[4.5]dec-2-en-8-one (Compound No. 26),
3-[3,4-bis(cyclopentyloxy)phenyl]-1,7-dioxa-2-azaspiro[4.4]non-2-ene (Compound No. 27),
3-[3,4-Bis(cyclopropylmethoxy)phenyl]-1,7-dioxa-2-azaspiro[4.4]non-2-ene (Compound No. 28),
3-[3-(cyclopentyloxy)-4-methoxyphenyl]-1,7-dioxa-2-azaspiro[4.4]non-2-en-4-ol (Compound No. 29),
(R)-3-[3-(cyclopentyloxy)-4-methoxyphenyl]-1,7-dioxa-2-azaspiro[4.4]non-2-ene (Compound No. 30),
3-[3-(Cyclopentyloxy)-4-methoxyphenyl]-8-(cyclopropylmethyl)-1-oxa-2,8-diazaspiro[4.5]dec-2-ene (Compound No. 31),
N-Benzyl-3-[3-(cyclopentyloxy)-4-methoxyphenyl]-1-oxa-2,8-diazaspiro[4.5]de-2-ene-8-carboxamide (Compound No. 32),
3-[3,4-Bis(benzyloxy)phenyl]-1,7-dioxa-2-azaspiro[4.4]non-2-ene (Compound No. 33),
4-(1,7-Dioxa-2-azaspiro[4.4]non-2-en-3-yl)benzene-1,2-diol (Compound No. 34).
7-Amino-3-[3-(cyclopentyloxy)-4-methoxyphenyl]-1-oxa-2,7-diazaspiro[4.4]non-2-en-6-one (Compound No. 35).
Ethyl 8-benzyl-3-[3-(cyclopentyloxy)-4-methoxyphenyl]-1-oxa-2,8-diazaspiro[4.5]dec-2-ene-4-carboxylate (Compound No. 36),
3-[3-(Cyclopentyloxy)-4-methoxyphenyl]-1-oxa-2-azaspiro[4.5]dec-2-ene-4-carboxylic acid (Compound no. 37),
8-Benzyl-3-[3-(cyclopentyloxy)-4-methoxyphenyl]-1-oxa-2,8-diazaspiro[4.5]dec-2-ene (Compound No. 38),
Ethyl 3-[3-(cyclopentyloxy)-4-methoxyphenyl]-1-oxa-2-azaspiro[4.5]dec-2-ene-4-carboxylate (Compound No. 39),
3-[3-(Difluoromethoxy)-4-methoxyphenyl]-1,7-dioxa-2-azaspiro[4.4]non-2-ene (Compound No. 40),
2-(Difluoromethoxy)-5-(1,7-dioxa-2-azaspiro[4.4]non-2-en-3-yl)phenol (Compound No. 41)
3-[3-(Cyclopentyloxy)-4-methoxyphenyl]-1-oxa-2,7-diazaspiro[4.4]non-2-en-6-one (Compound No. 42).
3-[3-(Cyclopentyloxy)-4-methoxyphenyl]-3a,6a-dimethyl-3aH-cyclopenta[d]isoxazole-4,6(5H,6aH)-dione (Compound No. 43),
3-[3-(Cyclopentyloxy)-4-methoxyphenyl]-3a,4,6,6a-tetrahydrofuro[3,4-d]isoxazole (Compound No. 44).
3-[3-(Cyclopentyloxy)-4-methoxyphenyl]-6,6a-dihydrofuro[3,4-d]isoxazol-4(3 aH)-one (Compound No. 45),
Tert-butyl [({3-[3-(cyclopentyloxy)-4-methoxyphenyl]-1-oxa-2-azaspiro[4.5]dec-2-en-8-yl}amino)carbonyl]carbamate (Compound No. 46),
N-{3-[3-(Cyclopentyloxy)-4-methoxyphenyl]-1-oxa-2-azaspiro[4.5]dec-2-en-8-yl}cyclopentanecarboxamide (Compound No. 47),
8-Acetyl-3-[3-(cyclopentyloxy)-4-methoxyphenyl]-1-oxa-2,8-diazaspiro[4.5]dec-2-ene (Compound No. 48),
8-(Cyclopentylcarbonyl)-3-[3-(cyclopentyloxy)-4-methoxyphenyl]-1-oxa-2,8-diazaspiro[4.5]dec-2-ene (Compound No. 49),
3-[3-(Cyclopentyloxy)-4-methoxyphenyl]-8-(2-piperidin-1-ylethyl)-1-oxa-2,8-diazaspiro[4.5]dec-2-ene (Compound No. 50),
3-(2,3-Dihydro-1,4-benzodioxin-6-yl)-1,7-dioxa-2-azaspiro[4.4]non-2-ene (Compound No. 51),
3-[3-(Cyclopentyloxy)-4-methoxyphenyl]-1,8-dioxa-2-azaspiro[4.5]dec-2-ene (Compound No. 52),
3-[3-(Cyclopentyloxy)-4-methoxyphenyl]-3 aH-cyclopenta[d]isoxazole-4,6(5H,6aH)-dione (Compound No. 53),
3-[3-(Cyclopentyloxy)-4-methoxyphenyl]-8-ethyl-1-oxa-2,8-diazaspiro[4.5]dec-2-ene (Compound No. 54),
3-[3-(Cyclopentyloxy)-4-methoxyphenyl]-8-vinyl-1-oxa-2-azaspiro[4.5]dec-2-en-8-ol (Compound No. 55),
3-[3-(Cyclopentyloxy)-4-methoxyphenyl]-3a,4,5,6,7,7a-hexahydro-1,2-benzisoxazole (Compound No. 56),
3-[3-(Cyclopentyloxy)-4-methoxyphenyl]-4,5,6,6a-tetrahydro-3 aH-cyclopenta[d]isoxazole (Compound No. 57),
N-{3-[3-(Cyclopentyloxy)-4-methoxyphenyl]-1-oxa-2-azaspiro[4.5]dec-2-en-8-yl}methanesulfonamide (Compound No. 58),
3-[3-(Cyclopentyloxy)-4-methoxyphenyl]-8-methyl-1-oxa-2-azaspiro[4.5]dec-2-en-8-ol (Compound No. 59).
3-[3-(Allyloxy)-4-methoxyphenyl]-1,7-dioxa-2-azaspiro[4.4]non-2-ene (Compound No. 60),
3-[3-(2-Chloroethoxy)-4-methoxyphenyl]-1,7-dioxa-2-azaspiro[4.4]non-2-ene (Compound No. 61),
2-(Cyclopentyloxy)-4-(1,7-dioxa-2-azaspiro[4.4]non-2-en-3-yl)phenol (Compound No. 62),
3-(4-Butoxy-3-isobutoxyphenyl)-1,7-dioxa-2-azaspiro[4.4]non-2-ene (Compound No. 63),
3-(3-Isobutoxy-4-propoxyphenyl)-1,7-dioxa-2-azaspiro[4.4]non-2-ene (Compound No. 64),
3-[3-Butoxy-4-(cyclopropylmethoxy)phenyl]-1,7-dioxa-2-azaspiro[4.4]non-2-ene (Compound No. 65),
3-(3-Butoxy-4-ethoxyphenyl)-1,7-dioxa-2-azaspiro[4.4]non-2-ene (Compound No. 66),
3-[3-Butoxy-4-(cyclohexyloxy)phenyl]-1,7-dioxa-2-azaspiro[4.4]non-2-ene (Compound No. 67),
3-[3-(Cyclohexylmethoxy)-4-ethoxyphenyl]-1,7-dioxa-2-azaspiro[4.4]non-2-ene (Compound No. 68),
3-[3-(Cyclohexylmethoxy)-4-isopropoxyphenyl]-1,7-dioxa-2-azaspiro[4.4]non-2-ene (Compound No. 69),
3-[4-Butoxy-3-(cyclohexylmethoxy)phenyl]-1,7-dioxa-2-azaspiro[4.4]non-2-ene (Compound No. 70),
3-(4-Isobutoxy-3-isopropoxyphenyl)-1,7-dioxa-2-azaspiro[4.4]non-2-ene (Compound No. 71),
3-(4-Butoxy-3-isopropoxyphenyl)-1,7-dioxa-2-azaspiro[4.4]non-2-one (Compound No. 72),
3-[4-(Cyclohexylmethoxy)-3-isopropoxyphenyl]-1,7-dioxa-2-azaspiro[4.4]non-2-ene (Compound No. 73),
3-[3-Isopropoxy-4-(2-morpholin-4-ylethoxy)phenyl]-1,7-dioxa-2-azaspiro[4.4]non-2-ene (Compound No. 74),
3-[3-(Cyclopropylmethoxy)-4-isopropoxyphenyl]-1,7-dioxa-2-azaspiro[4.4]non-2-ene (Compound No. 75),
3-[3-(Cyclopropylmethoxy)-4-(2-morpholin-4-ylethoxy)phenyl]-1,7-dioxa-2-azaspiro[4.4]non-2-ene (Compound No. 76),
3-[4-Butoxy-3-(cyclopropylmethoxy)phenyl]-1,7-dioxa-2-azaspiro[4.4]non-2-ene (Compound No. 77),
3-[3-(Cyclopropylmethoxy)-4-isopropoxyphenyl]-1,7-dioxa-2-azaspiro[4.4]non-2-ene (Compound No. 78),
3-(3-Isobutoxy-4-isopropoxyphenyl)-1,7-dioxa-2-azaspiro[4.4]non-2-ene (Compound No. 79),
3-[4-(Cyclopropylmethoxy)-3-isobutoxyphenyl]-1,7-dioxa-2-azaspiro[4.4]non-2-ene (Compound No. 80),
3-[4-(cyclohexyloxy)-3-(cyclopentyloxy)phenyl]-1,7-dioxa-2-azaspiro[4.4]non-2-ene (Compound No. 81)
3-[4-(Cyclohexylmethoxy)-3-(cyclopentyloxy)phenyl]-1,7-dioxa-2-azaspiro[4.4]non-2-ene (Compound No. 82),
3-[4-(Cyclopropylmethoxy)-3-(cyclopentyloxy)phenyl]-1,7-dioxa-2-azaspiro[4.4]non-2-ene (Compound No. 83),
3-[3-(Cyclopentyloxy)-4-isobutoxyphenyl]-1,7-dioxa-2-azaspiro[4.4]non-2-ene (Compound No. 84),
3-[3-(Cyclopentyloxy)-4-ethoxyphenyl]-1,7-dioxa-2-azaspiro[4.4]non-2-ene (Compound No. 85),
3-[3-(Cyclopropylmethoxy)-4-ethoxyphenyl]-1,7-dioxa-2-azaspiro[4.4]non-2-ene (Compound No. 86),
3-[4-(Cyclopentyloxy)-3-isobutoxyphenyl]-1,7-dioxa-2-azaspiro[4.4]non-2-ene (Compound No. 87),
3-[3-Isopropoxy-4-(2-morpholin-4-ylethoxy)phenyl]-1,7-dioxa-2-azaspiro[4.4]non-2-ene (Compound No. 88),
3-(4-Ethoxy-3-isobutoxyphenyl)-1,7-dioxa-2-azaspiro[4.4]non-2-ene (Compound No. 89)
3-[3-(Cyclopentyloxy)-4-propoxyphenyl]-1,7-dioxa-2-azaspiro[4.4]non-2-ene (Compound No. 90),
3-[4-Butoxy-3-(cyclopentyloxy)phenyl]-1,7-dioxa-2-azaspiro[4.4]non-2-ene (Compound No. 91),
3-[3-(Cyclopentyloxy)-4-isopropoxyphenyl]-1,7-dioxa-2-azaspiro[4.4]non-2-ene (Compound No. 92),
3-[3-(Cyclopentyloxy)-4-(cycloheptyloxy)phenyl]-1,7-dioxa-2-azaspiro[4.4]non-2-ene (Compound No. 93),
3-[3-(Cyclopentyloxy)-4-(2-morpholin-4-ylethoxy)phenyl]-1,7-dioxa-2-azaspiro[4.4]non-2-ene (Compound No. 94),
3-[4-(Cyclohexylmethoxy)-3-isobutoxyphenyl]-1,7-dioxa-2-azaspiro[4.4]non-2-ene (Compound No. 95),
3-[4-(Cyclohexylmethoxy)-3-(cyclopropylmethoxy)phenyl]-1,7-dioxa-2-azaspiro[4.4]non-2-ene (Compound No. 96),
3-[3-(Cyclopropylmethoxy)-4-propoxyphenyl]-1,7-dioxa-2-azaspiro[4.4]non-2-ene (Compound No. 97),
3-[4-(Cyclopentyloxy)-3-(cyclopropylmethoxy)phenyl]-1,7-dioxa-2-azaspiro[4.4]non-2-ene (Compound No. 98),
3-[4-(Cyclopropylmethoxy)-3-isopropoxyphenyl]-1,7-dioxa-2-azaspiro[4.4]non-2-ene (Compound No. 99),
3-[4-(Cyclopentyloxy)-3-isopropoxyphenyl]-1,7-dioxa-2-azaspiro[4.4]non-2-ene (Compound No. 100),
3-(3-Isopropoxy-4-propoxyphenyl)-1,7-dioxa-2-azaspiro[4.4]non-2-ene (Compound No. 101),
3-(4-Ethoxy-3-isopropoxyphenyl)-1,7-dioxa-2-azaspiro[4.4]non-2-ene (Compound No. 102),
3-[3-Butoxy-4-(2-morpholin-4-ylethoxy)phenyl]-1,7-dioxa-2-azaspiro[4.4]non-2-ene (Compound No. 103),
3-[3-Butoxy-4-(cyclopentyloxy)phenyl]-1,7-dioxa-2-azaspiro[4.4]non-2-ene (Compound No. 104),
3-(3-Butoxy-4-propoxyphenyl)-1,7-dioxa-2-azaspiro[4.4]non-2-ene (Compound No. 105),
3-(3-Butoxy-4-isopropoxyphenyl)-1,7-dioxa-2-azaspiro[4.4]non-2-ene (Compound No. 106),
3-[3-(Cyclohexylmethoxy)-4-propoxyphenyl]-1,7-dioxa-2-azaspiro[4.4]non-2-ene (Compound No. 107),
3-[3-(Cyclohexylmethoxy)-4-isobutoxyphenyl]-1,7-dioxa-2-azaspiro[4.4]non-2-ene (Compound No. 108),
3-[3-(Cyclohexylmethoxy)-4-(cyclopentyloxy)phenyl]-1,7-dioxa-2-azaspiro[4.4]non-2-ene (Compound No. 109),
3-[3-(Cyclohexylmethoxy)-4-(cyclopropylmethoxy)phenyl]-1,7-dioxa-2-azaspiro[4.4]non-2-ene (Compound No. 110),
3-[4-(Cyclohexylmethoxy)-3-propoxyphenyl]-1,7-dioxa-2-azaspiro[4.4]non-2-ene (Compound No. 111),
3-[4-(Cyclopropylmethoxy)-3-propoxyphenyl]-1,7-dioxa-2-azaspiro[4.4]non-2-ene (Compound No. 112),
3-[4-(Cyclopentyloxy)-3-propoxyphenyl]-1,7-dioxa-2-azaspiro[4.4]non-2-ene (Compound No. 113),
3-[4-(3-Isobutoxy)-3-propoxyphenyl]-1,7-dioxa-2-azaspiro[4.4]non-2-ene (Compound No. 114),
3-[3-(Cycloheptyloxy)-4-(cyclopropylmethoxy)phenyl]-1,7-dioxa-2-azaspiro[4.4]non-2-ene (Compound No. 115),
3-[3-(Cycloheptyloxy)-4-propoxyphenyl]-1,7-dioxa-2-azaspiro[4.4]non-2-ene (Compound No. 116),
3-[4-Butoxy-3-(cycloheptyloxy)phenyl]-1,7-dioxa-2-azaspiro[4.4]non-2-ene (Compound No. 117),
3-[3-(Cycloheptyloxy)-4-isopropoxyphenyl]-1,7-dioxa-2-azaspiro[4.4]non-2-ene (Compound No. 118),
3-[3-(Cycloheptyloxy)-4-(cyclopentyloxy)phenyl]-1,7-dioxa-2-azaspiro[4.4]non-2-ene (Compound No. 119),
3-(3-Ethoxy-4-propoxyphenyl)-1,7-dioxa-2-azaspiro[4.4]non-2-ene (Compound No. 120),
3-[4-(Cycloheptyloxy)-3-ethoxyphenyl]-1,7-dioxa-2-azaspiro[4.4]non-2-ene (Compound No. 121),
3-[4-(Cyclopropylmethoxy)-3-ethoxyphenyl]-1,7-dioxa-2-azaspiro[4.4]non-2-ene (Compound No. 122),
3-[4-(Cyclohexylmethoxy)-3-ethoxyphenyl]-1,7-dioxa-2-azaspiro[4.4]non-2-ene (Compound No. 123),
(S)-3-[3-(cyclopentyloxy)-4-methoxyphenyl]-1,7-dioxa-2-azaspiro[4.4]non-2-ene (Compound No. 124
3-(3-Butoxy-4-isobutoxyphenyl)-1,7-dioxa-2-azaspiro[4.4]non-2-ene (Compound No. 125),
3-(3-Ethoxy-4-isopropoxyphenyl)-1,7-dioxa-2-azaspiro[4.4]non-2-ene (Compound No. 126),
3-[4-(Cyclopentyloxy)-3-ethoxyphenyl]-1,7-dioxa-2-azaspiro[4.4]non-2-ene (Compound No. 127),
3-(4-Butoxy-3-ethoxyphenyl)-1,7-dioxa-2-azaspiro[4.4]non-2-ene (Compound No. 128),
3-(3-Ethoxy-4-isobutoxyphenyl)-1,7-dioxa-2-azaspiro[4.4]non-2-ene (Compound No. 129),
3-[3-(Cycloheptyloxy)-4-isobutoxyphenyl]-1,7-dioxa-2-azaspiro[4.4]non-2-ene (Compound No. 130),
3-[3-(Cycloheptyloxy)-4-(cyclopentyloxy)phenyl]-1,7-dioxa-2-azaspiro[4.4]non-2-ene (Compound No. 131),
3-[3-(Cycloheptyloxy)-4-ethoxyphenyl]-1,7-dioxa-2-azaspiro[4.4]non-2-ene (Compound No. 132),
3-(4-Butoxy-3-propoxyphenyl)-1,7-dioxa-2-azaspiro[4.4]non-2-ene (Compound No. 133),
3-(4-Ethoxy-3-propoxyphenyl)-1,7-dioxa-2-azaspiro[4.4]non-2-ene (Compound No. 134),
3-[4-(Morpholin-4-ylethoxy)-3-propoxyphenyl]-1,7-dioxa-2-azaspiro[4.4]non-2-ene (Compound No. 135),
3-(4-Isopropoxy-3-propoxyphenyl)-1,7-dioxa-2-azaspiro[4.4]non-2-ene (Compound No. 136),
2-[5-(1,7-Dioxa-2-azaspiro[4.4]non-2-en-3-yl)-2-methoxyphenoxy]cyclopentanol (Compound No. 137).
N-{3-[3-(Cyclopentyloxy)-4-methoxyphenyl]-1-oxa-2-azaspiro[4.5]dec-2-en-8-yl}-2-fluorobenzamide (Compound No. 138),
N-{3-[3-(cyclopentyloxy)-4-methoxyphenyl]-1-oxa-2-azaspiro[4.5]dec-2-en-8-yl}benzamide (Compound No. 139).
3-[3-(Cyclopentyloxy)-4-methoxyphenyl]-4,5,6,6a-tetrahydro-3 aH-pyrrolo[3,4-d]isoxazole (Compound No. 140)
7-(Cyclopentylcarbonyl)-3-[3-(cyclopentyloxy)-4-methoxyphenyl]-1-oxa-2,7-diazaspiro[4.5]dec-2-ene (Compound No. 141),
Tert-butyl 3-[3-(cyclopentyloxy)-4-methoxyphenyl]-3a,4,6,6a-tetrahydro-5H-pyrrolo[3,4-d]isoxazole-5-carboxylate (Compound No. 142),
3-[3-(Cyclopentyloxy)-4-methoxyphenyl]-1-oxa-2,8-diazaspiro[4.5]dec-2-ene-8-carboxamide (Compound No. 143),
N-Butyl-3-[3-(cyclopentyloxy)-4-methoxyphenyl]-1-oxa-2,7-diazaspiro[4.5]dec-2-ene-7-carboxamide (Compound No. 144),
3-[3-(Cyclopentyloxy)-4-methoxyphenyl]-7-(methylsulfonyl)-1-oxa-2,7-diazaspiro[4.5]dec-2-ene (Compound No. 145).
3-[4-Methoxy-3-(pyridin-3-ylmethoxy)phenyl]-1,7-dioxa-2-azaspiro[4.4]non-2-ene (Compound No. 146),
5-Acetyl-3-[3-(cyclopentyloxy)-4-methoxyphenyl]-4,5,6,6a-tetrahydro-3aH-pyrrolo[3,4-d]isoxazole (Compound No. 147).
3-[3-(Cyclopentyloxy)-4-methoxyphenyl]-5-(methylsulfonyl)-4,5,6,6a-tetrahydro-3aH-pyrrolo[3,4-d]isoxazole (Compound No. 148).
4-Bromo-3-[3-(cyclopentyloxy)-4-methoxyphenyl]-1,7-dioxa-2-azaspiro[4.4]non-2-ene (Compound No. 149)
3-[3-(Cyclopentyloxy)-4-methoxyphenyl]-3a,5,6,7a-tetrahydro-1,2-benzisoxazol-7(4H)-one (Compound No. 150).
3-[4-(Difluoromethoxy)-3-(2,3-dihydro-1H-inden-2-yloxy)phenyl]-1,7-dioxa-2-azaspiro[4.4]non-2-ene (Compound No. 151),
3-[4-(Cyclopentyloxy)-3-(2,3-dihydro-1H-inden-2-yloxy)phenyl]-1,7-dioxa-2-azaspiro[4.4]non-2-ene (Compound No. 152),
3-[4-Butoxy-3-(2,3-dihydro-1H-inden-2-yloxy)phenyl]-1,7-dioxa-2-azaspiro[4.4]non-2-ene (Compound No. 153),
3-(3-{[3-(Benzyloxy)cyclopentyl]oxy}-4-methoxyphenyl)-1,7-dioxa-2-azaspiro[4.4]non-2-ene (Compound No. 154),
7-Acetyl-3-[3-(cyclopentyloxy)-4-methoxyphenyl]-1-oxa-2,7-diazaspiro[4.5]dec-2-ene (Compound No. 155),
3-[4-Methoxy-3-(pyridin-2-ylmethoxy)phenyl]-1,7-dioxa-2-azaspiro[4.4]non-2-ene (Compound No. 156),
3-[3-(2,3-Dihydro-1H-inden-2-yloxy)-4-ethoxyphenyl]-1,7-dioxa-2-azaspiro[4.4]non-2-ene (Compound No. 157),
3-[3-(2,3-Dihydro-1H-inden-2-yloxy)-4-propoxyphenyl]-1,7-dioxa-2-azaspiro[4.4]non-2-ene (Compound No. 158),
3-[4-(Cyclopropylmethoxy)-3-(2,3-dihydro-1H-inden-2-yloxy)phenyl]-1,7-dioxa-2-azaspiro[4.4]non-2-ene (Compound No. 159),
3-[3-(2,3-Dihydro-1H-inden-2-yloxy)-4-isopropoxyphenyl]-1,7-dioxa-2-azaspiro[4.4]non-2-ene (Compound No. 160),
2-(2,3-Dihydro-1H-inden-2-yloxy)-4-(1,7-dioxa-2-azaspiro[4.4]non-2-en-3-yl)phenol (Compound No. 161),
N-cyclopropyl-2-[5-(1,7-dioxa-2-azaspiro[4.4]non-2-en-3-yl)-2-methoxyphenoxy]acetamide (Compound No. 162),
Hydrochloride salt of 3-[4-methoxy-3-(piperidin-3-yloxy)phenyl]-1,7-dioxa-2-azaspiro[4.4]non-2-ene (Compound No. 163),
2-[5-(1,7-Dioxa-2-azaspiro[4.4]non-2-en-3-yl)-2-methoxyphenoxy]acetamide (Compound No. 164),
Ethyl [5-(1,7-dioxa-2-azaspiro[4.4]non-2-en-3-yl)-2-methoxyphenoxy]acetate (Compound No. 165),
[5-(1,7-Dioxa-2-azaspiro[4.4]non-2-en-3-yl)-2-methoxyphenoxy]acetonitrile (Compound No. 166),
3-{3-[(2,6-Dichloropyridin-4-yl)methoxy]-4-methoxyphenyl}-1,7-dioxa-2-azaspiro[4.4]non-2-ene (Compound No. 167).

3. A pharmaceutical composition comprising a therapeutically effective amount of a compound of claim 1 together with a pharmaceutically acceptable carrier, excipient or diluent.

4. A method of treating AIDS, asthma, arthritis, bronchitis, chronic obstructer pulmonary disease (COPD), psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis or other inflammatory diseases in an animal or human comprising administering to said animal or human a therapeutically effective amount of a compound of claim 1.

5. A method of preventing, inhibiting or suppressing inflammatory condition in an animal or human comprising administering to said animal or human a therapeutically effective amount of a compound of claim 1.

6. (canceled)

7. (canceled)

8. (canceled)

9. (canceled)

10. (canceled)

11. (canceled)

12. (canceled)

13. (canceled)

14. (canceled)

15. (canceled)

16-26. (canceled)

27. (canceled)

28. (canceled)

29. (canceled)

30. A method for preparing a compound of Formula LXXX, its pharmaceutically acceptable salts, pharmaceutically acceptable solvates, enantiomers, diastereomers or N-oxides wherein the method comprises the steps of:
a. reacting a compound of Formula LXXV 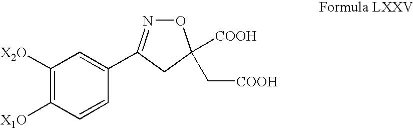
with a compound of Formula LXXVI
Q Formula LXXVI
to give a compound of Formula LXXVII, 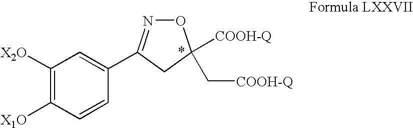
b. protecting a compound of Formula LXXVII with a compound of Formula P′-OH to give a compound of Formula LXXVIII, 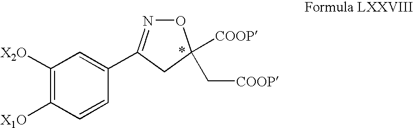
c. reducing a compound of Formula LXVIII to give a compound of Formula LXXIX, 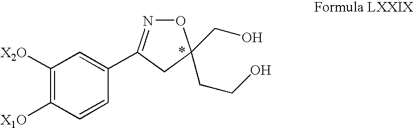
d. cyclizing a compound of Formula LXXIX to give a compound of Formula LXXX, 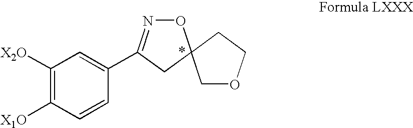
wherein
X₁ and X₂ is hydrogen, alkyl, cycloalkyl, alkaryl, alkenyl, cycloalkylalkyl, heteroaryl, heterocyclyl, heteroarylalkyl, heterocyclylalkyl, —(CH₂)_{g}C(═O)NRₓR_{y} or —(CH₂)_{g1}C(═O)OR₃;
g is an integer from 0-3;
g₁ is an integer from 1-3;
X₁ and X₂ together can optionally form a cyclic ring fused with the ring A shown in Formula I, the ring containing 3-5 carbon atoms within the ring and having 2-3 heteroatoms selected from the group consisting of N, O and S;
R₃ is alkyl, cycloalkyl or heterocyclyl;
Rₓ and R_{y} each independently is hydrogen, alkyl, C₃-C₆ alkenyl, C₃-C₆ alkynyl, carboxy, cycloalkyl, —S(O)ₘR₅, aryl, alkaryl, heteroaryl, heterocyclyl, heteroarylalkyl, and heterocyclylalkyl;
m is an integer between 0-2;
R₅ is hydrogen, alkyl, alkenyl, alkynyl, aryl, cycloalkyl, alkaryl, heteroaryl, heteroarylalkyl, heterocyclyl or heterocyclylalkyl;
Q is a chiral resolving agent selected from L-Ephederine, D-Ephederine, Brucine, (1S,2R)
(−)-cis-1-amino-2-indanol, (1R2S) (+)-cis-1-amino-2-indanol, (1R,2R)-(−)-1,2-diamino cyclohexane or (1S,2S)-(+)-1,2-diamino cyclohexane or α-methylbenzylamine; and
P′ is alkyl.
